(19) United States
(12) Patent Application Publication
(10) Pub. No.: US 2024/0245354 A1
(43) Pub. Date: Jul. 25, 2024

(54) REMOTE CARDIOTOXICITY MONITORING

(71) Applicant: ZOLL Medical Corporation, Chelmsford, MA (US)

(72) Inventors: Bahar Davoudi, Pittsburgh, PA (US); Kent J Volosin, Mars, PA (US)

(21) Appl. No.: 18/407,810

(22) Filed: Jan. 9, 2024

Related U.S. Application Data
(60) Provisional application No. 63/479,862, filed on Jan. 13, 2023.

(51) Int. Cl. A61B 5/00; A61B 5/0205; G16H 20/30; G16H 40/67

(52) U.S. Cl. CPC A61B 5/6804; A61B 5/0006; A61B 5/0022; A61B 5/0205; G16H 20/30; G16H 40/67; A61B 5/256

(57) ABSTRACT

A wearable cardiotoxicity monitoring system for monitoring cardiotoxicity biosignal markers in oncology patients is provided. The system includes a wearable cardiotoxicity monitoring device configured for long-term, continuous wear by a patient, including externally applied biosignal sensors configured to sense biosignal(s) from the patient and a controller. The externally applied biosignal sensors include ECG electrodes and at least one non-ECG physiological sensor. The controller is configured to transmit biosignal-based data based on the sensed biosignal(s) to a remote server. The system further includes the remote server, including a memory and a processor. The processor is configured to receive the biosignal-based data, analyze the biosignal-based data to identify cardiotoxicity biosignal marker(s) associated with heart failure caused by chemotherapy and/or radiation therapy cardiotoxicity, determine a current status of cardiotoxicity in the patient based on the cardiotoxicity biosignal marker(s), and generate an output based on the current status of cardiotoxicity.

### CROSS-REFERENCE TO RELATED APPLICATION

This nonprovisional application claims priority to U.S. Provisional Patent Application Ser. No. 63/479,862, filed on Jan. 13, 2023, titled “REMOTE CARDIOTOXICITY MONITORING,” the entirety of which is hereby incorporated by reference.

### BACKGROUND

The present disclosure relates to a wearable cardiotoxicity monitoring system configured to remotely monitor oncology patients including cancer survivors.

When a patient is diagnosed with cancer, they may be prescribed one of a number of different treatments. Among these potential treatments is chemotherapy. With chemotherapy, the patient takes a regimen of drugs that typically target fast-growing cells in the patient's body, given that cancer cells usually multiply more quickly than most other types of cells. Examples of chemotherapeutic agents include alkylating agents, plant alkaloids, antimetabolites, anthracyclines, topoisomerase inhibitors, and corticosteroids. Another potential treatment for cancer is radiation therapy. When a patient receives radiation therapy, high-energy x-rays or other particles are typically directed at areas of the patient's body containing cancer. These high radiation doses can destroy cancer cells in these areas of the patient's body. However, in addition to killing cancerous cells and tissues in the patient's body, chemotherapy and radiation therapy may have undesired side effects and toxicities in patients receiving treatment.

### SUMMARY

In one or more examples, a wearable cardiotoxicity monitoring system for monitoring cardiotoxicity biosignal markers in oncology patients is provided. The wearable cardiotoxicity monitoring system includes a wearable cardiotoxicity monitoring device configured for long-term, continuous wear by a patient. The wearable cardiotoxicity monitoring device includes a plurality of externally applied biosignal sensors configured to sense one or more biosignals from the patient. The plurality of externally applied biosignal sensors includes a plurality of electrocardiogram (ECG) electrodes configured to sense one or more electrical signals indicative of ECG activity from a skin surface of the patient, and at least one non-ECG physiological sensor configured to sense a non-ECG biosignal of the patient. The wearable cardiotoxicity monitoring device further includes a controller operationally coupled to the plurality of externally applied biosignal sensors. The controller is configured to transmit biosignal-based data based on the one or more sensed biosignals to a remote server. The wearable cardiotoxicity monitoring system further includes the remote server in communication with the wearable cardiotoxicity monitoring device. The remote server includes a memory implemented in a non-transitory media and a processor in communication with the memory. The processor is configured to receive the biosignal-based data from the wearable cardiotoxicity monitoring device, analyze the biosignal-based data from the wearable cardiotoxicity monitoring device to identify at least one cardiotoxicity biosignal marker associated with heart failure caused by at least one of chemotherapy cardiotoxicity or radiation therapy cardiotoxicity, determine a current status of cardiotoxicity in the patient based on the at least one cardiotoxicity biosignal marker, and generate an output based on the current status of cardiotoxicity in the patient.

Implementations of the wearable cardiotoxicity monitoring device can include one or more of the following features. The wearable cardiotoxicity monitoring device further includes a garment configured to be worn around the patient's torso. At least a portion of the plurality of externally applied biosignal sensors are configured to be removably mounted into the garment. At least a portion of the plurality of externally applied biosignal sensors are permanently integrated into the garment. The wearable cardiotoxicity monitoring device further includes one or more therapy electrodes configured to deliver one or more therapeutic shocks to the patient. The controller is further configured to detect a treatable cardiac arrhythmia in the patient and generate the one or more therapeutic shocks for delivery to the patient via the one or more therapy electrodes based on detecting the treatable cardiac arrhythmia in the patient.

The wearable cardiotoxicity monitoring device further includes a removable adhesive patch configured to be adhered to the patient's torso. At least a portion of the externally applied biosignal sensors are integrated into the adhesive patch. At least a portion of the externally applied biosignal sensors are configured to be removably mounted onto the adhesive patch. The wearable cardiotoxicity monitoring device further includes a portable gateway configured to facilitate the communication between the wearable cardiotoxicity monitoring device and the remote server. The wearable cardiotoxicity monitoring system of claim 1, wherein the controller further configured to record at least one symptom event based on an input by the patient and transmit an indication of the at least one symptom event to the remote server. The output is further based on the indication of the at least one symptom event.

The at least one non-ECG physiological sensor includes a cardiovibration sensor configured to sense cardiovibrations of the patient. The at least one non-ECG physiological sensor includes a respiration sensor configured to sense biosignals indicative of a respiration rate of the patient. The at least one non-ECG physiological sensor includes a radiofrequency (RF) sensor configured to receive RF-based biosignals indicative of a thoracic fluid level of the patient. The at least one non-ECG physiological sensor includes a blood pressure sensor configured to sense a biosignals indicative of a blood pressure of the patient.

The biosignal-based data includes an ECG of the patient, and the at least one cardiotoxicity biosignal marker includes at least one ECG marker associated with heart failure caused by the at least one of chemotherapy cardiotoxicity or radiation therapy cardiotoxicity. The at least one ECG marker associated with heart failure caused by the at least one of chemotherapy cardiotoxicity or radiation therapy cardiotoxicity includes a PR interval duration. The at least one ECG marker associated with heart failure caused by the at least one of chemotherapy cardiotoxicity or radiation therapy cardiotoxicity includes a QRS complex duration. The at least one ECG marker associated with heart failure caused by the at least one of chemotherapy cardiotoxicity or radiation therapy cardiotoxicity includes a QT interval duration.

The biosignal-based data includes cardiovibrational data for the patient. The at least one cardiotoxicity biosignal marker includes at least one cardiovibrational biomarker for the patient. The at least one cardiovibrational biomarker for the patient includes at least one of an S1, S2, S3, or S4 cardiovibrational biomarker. The biosignal-based data further includes an ECG of the patient. The at least one cardiotoxicity biosignal marker includes a timing between a predetermined ECG marker of the ECG of the patient and a predetermined cardiovibrational biomarker of the cardiovibrational data for the patient. The predetermined ECG marker of the ECG includes a P wave, a Q wave, an R wave, an S wave, or a T wave. The predetermined cardiovibrational biomarker of the cardiovibrational data includes an S1, S2, S3, or S4 cardiovibrational biomarker.

The biosignal-based data includes an ECG of the patient, and the at least one cardiotoxicity biosignal marker includes a heart rate of the patient. The biosignal-based data includes respiration data, and the at least one cardiotoxicity biosignal marker includes a respiration rate of the patient. The biosignal-based data includes at least one RF-based measurement associated with a thoracic fluid level in the patient, and the at least one cardiotoxicity biosignal marker includes the thoracic fluid level in the patient. The thoracic fluid level includes a relative thoracic fluid level. The thoracic fluid level includes an absolute thoracic fluid level. The biosignal-based data includes blood pressure data, and the at least one cardiotoxicity biosignal marker includes a blood pressure of the patient.

The wearable cardiotoxicity monitoring device further includes at least one additional externally applied sensor configured to sense one or more additional signals associated with the patient. The controller is further configured to transmit additional sensor data based on the one or more additional signals associated with the patient to the remote server. The processor is further configured to receive the additional sensor data from the wearable cardiotoxicity monitoring device, analyze the additional sensor data from the wearable cardiotoxicity monitoring device to identify at least one additional cardiotoxicity marker associated with heart failure caused by the at least one of chemotherapy cardiotoxicity or radiation therapy cardiotoxicity, and determine the current status of cardiotoxicity in the patient further based on the at least one additional cardiotoxicity marker. The at least one additional externally applied sensor includes an accelerometer configured to sense one or more motion signals associated with the patient. The additional sensor data based on the one or more additional signals includes accelerometer data based on the one or more motion signals. The at least one additional cardiotoxicity marker includes an activity level of the patient. The at least one additional cardiotoxicity marker includes a body position of the patient.

The processor is configured to analyze the biosignal-based data from the wearable cardiotoxicity monitoring device to identify the at least one cardiotoxicity biosignal marker associated with heart failure by sampling the biosignal-based data to identify the at least one cardiotoxicity biosignal marker associated with heart failure. The processor is configured to determine the current status of cardiotoxicity in the patient based on the at least one cardiotoxicity biosignal marker by applying a statistical analysis to the at least one cardiotoxicity biosignal marker to determine at least one representative cardiotoxicity biosignal marker, and determining the current status of cardiotoxicity in the patient using the at least one representative cardiotoxicity biosignal marker. Applying the statistical analysis to the at least one cardiotoxicity biosignal marker to determine the at least one representative cardiotoxicity biosignal marker includes determining at least one of a mean, median, mode, highest value, or lowest value of the at least one cardiotoxicity biosignal marker. The processor is configured to determine the current status of cardiotoxicity in the patient based on the at least one cardiotoxicity biosignal marker by determining whether the at least one cardiotoxicity biosignal marker transgresses at least one predetermined threshold. Determining whether the at least one cardiotoxicity biosignal marker transgresses the at least one predetermined threshold includes determining whether a first cardiotoxicity biomarker transgresses a first predetermined threshold and determining whether a second cardiotoxicity biomarker transgresses a second predetermined threshold. Determining whether the at least one cardiotoxicity biosignal marker transgresses the at least one predetermined threshold includes determining whether a first cardiotoxicity biomarker transgresses a first predetermined threshold associated with a first, lower heart failure severity and whether the first cardiotoxicity biomarker transgresses a second predetermined threshold associated with a second, higher heart failure severity. The processor is configured to determine the current status of cardiotoxicity in the patient based on the at least one cardiotoxicity biosignal marker using a cardiotoxicity evaluation function, wherein the at least one cardiotoxicity biosignal marker includes at least one input to the cardiotoxicity evaluation function. The cardiotoxicity evaluation function includes at least one of a linear function, an nth-order polynomial function, a logarithmic function, an exponential function, or a power function.

The controller is further configured to, before the patient begins at least one of chemotherapy or radiation therapy, transmit baseline biosignal-based data based on the one or more sensed biosignals to the remote server. The processor is further configured to receive the baseline biosignal-based data from the wearable cardiotoxicity monitoring device, analyze the baseline biosignal-based data from the wearable cardiotoxicity monitoring device to identify at least one baseline cardiotoxicity biosignal marker associated with a baseline heart failure status before application of at least one of chemotherapy or radiation therapy to the patient, and determine a baseline status of cardiotoxicity in the patient based on the at least one baseline cardiotoxicity biosignal marker. The processor is further configured to generate a baseline output based on the baseline status of cardiotoxicity in the patient. The baseline output includes a cardiac risk assessment for the patient based on the determined baseline status of cardiotoxicity.

The controller is further configured to, after the patient finishes at least one of chemotherapy or radiation therapy, transmit post-therapy biosignal-based data based on the one or more sensed biosignals to the remote server. The processor is further configured to receive the post-therapy biosignal-based data from the wearable cardiotoxicity monitoring device, analyze the post-therapy biosignal-based data from the wearable cardiotoxicity monitoring device to identify at least one post-therapy cardiotoxicity biosignal marker associated with heart failure caused by the at least one of chemotherapy cardiotoxicity or radiation therapy cardiotoxicity, and determine a post-therapy status of cardiotoxicity in the patient based on the at least one post-therapy cardiotoxicity biosignal marker. The processor is further configured to generate a post-therapy output based on the post-therapy status of cardiotoxicity in the patient.

The output includes an alert transmitted to a caretaker of the patient. The output includes a report transmitted to a caretaker of the patient, the report including the current status of cardiotoxicity in the patient. The output includes a cardiac risk assessment for the patient. The processor is configured to determine the current status of cardiotoxicity in the patient by generating a trend based on the at least one cardiotoxicity biosignal marker. The output includes the trend based on the at least one cardiotoxicity biosignal marker.

In one or more examples, a method for monitoring cardiotoxicity biosignal markers in oncology patients is performed. The method includes receiving biosignal-based data from a wearable cardiotoxicity monitoring device associated with a patient, analyzing the biosignal-based data from the wearable cardiotoxicity monitoring device to identify at least one cardiotoxicity biosignal marker associated with heart failure in the patient caused by at least one of chemotherapy cardiotoxicity or radiation therapy cardiotoxicity, determining a current status of cardiotoxicity in the patient based on the at least one cardiotoxicity biosignal marker, and generating an output based on the current status of cardiotoxicity in the patient.

In one or more examples, a non-transitory computer-readable medium storing sequences of instructions executable by at least one processor is provided. The sequences of instructions instruct the at least one processor to receive biosignal-based data from a wearable cardiotoxicity monitoring device associated with a patient, analyze the biosignal-based data from the wearable cardiotoxicity monitoring device to identify at least one cardiotoxicity biosignal marker associated with heart failure in the patient caused by at least one of chemotherapy cardiotoxicity or radiation therapy cardiotoxicity, determine a current status of cardiotoxicity in the patient based on the at least one cardiotoxicity biosignal marker, and generate an output based on the current status of cardiotoxicity in the patient.

### BRIEF DESCRIPTION OF THE DRAWINGS

Various aspects of at least one example are discussed below with reference to the accompanying figures, which are not intended to be drawn to scale. The figures are included to provide an illustration and a further understanding of the various aspects and examples, and are incorporated in and constitute a part of this specification, but are not intended to limit the scope of the disclosure. The drawings, together with the remainder of the specification, serve to explain principles and operations of the described and claimed aspects and examples. In the figures, each identical or nearly identical component that is illustrated in various figures is represented by a like numeral. For purposes of clarity, not every component may be labeled in every figure.

FIG. 1 depicts an example wearable cardiotoxicity monitoring system including a wearable cardiotoxicity monitoring device.

FIG. 2 depicts an example adhesive patch of a wearable cardiotoxicity monitoring device.

FIG. 3 depicts an example cardiotoxicity monitoring unit of a wearable cardiotoxicity monitoring device.

FIG. 4 depicts an example of using an adhesive patch with a cardiotoxicity monitoring unit.

FIG. 5 depicts an example electronic architecture for a cardiotoxicity monitoring unit.

FIG. 6 depicts another example wearable cardiotoxicity monitoring system including a wearable cardiotoxicity monitoring device.

FIG. 7 depicts an example garment-based monitoring device of a wearable cardiotoxicity monitoring device.

FIG. 8 depicts an example electronic architecture for a garment-based monitoring device.

FIG. 9 depicts an example process flow for monitoring cardiotoxicity biosignal markers in oncology patients.

FIG. 10 depicts an example process flow for using a patient's PR intervals to determine the current status of cardiotoxicity in a patient.

FIG. 11 depicts an example process flow for using a patient's P waves and QRS complexes to determine the current status of cardiotoxicity in a patient.

FIG. 12 depicts an example process flow for using a patient's QRS complexes to determine the current status of cardiotoxicity in a patient.

FIG. 13 depicts an example process flow for using a patient's QT intervals to determine the current status of cardiotoxicity in a patient.

FIG. 14 depicts an example process flow for using a patient's T waves to determine the current status of cardiotoxicity in a patient.

FIG. 15A depicts an example screen that may be displayed to a patient.

FIG. 15B depicts another example screen that may be displayed to a patient.

FIG. 15C depicts another example screen that may be displayed to a patient.

FIG. 16 depicts an example process flow for monitoring baseline cardiotoxicity biosignal markers in oncology patients.

FIG. 17 depicts an example process flow for monitoring post-therapy cardiotoxicity biosignal markers in oncology patients.

FIG. 18 depicts an example component view of a cardiotoxicity monitoring unit.

FIG. 19 depicts another example wearable cardiotoxicity monitoring device.

FIG. 20 depicts another example wearable cardiotoxicity monitoring device.

FIG. 21 depicts another example wearable cardiotoxicity monitoring device.

### DETAILED DESCRIPTION

Wearable cardiotoxicity monitoring systems implementing the devices, methods, and techniques disclosed herein can be used in clinical care settings to monitor patients receiving oncology treatments. Such oncology treatments may include, for example, the administration of chemotherapeutic agents and/or radiation therapy designed to target and kill cancerous cells and tissues. However, as a side effect of oncology treatments, patients may undergo mild to severe side effects. In some cases, these side effects may include cardiotoxicity to the heart of the patient. For instance, chemotherapy and/or radiation therapy may create conduction issues in the patient's heart (e.g., by creating fibrous tissues). These conduction issues may weaken the contractions of the patient's heart, cause the patient's heart to beat more slowly, increase the likelihood that the patient will experience a life-threatening arrhythmia, and/or the like. Since the 1990s, there has been a steady decline in oncology-related mortality mirrored by a steady increase in cancer survivors. In this context, oncology treatment-related side effects have gained significance. Management of cancer therapy-related cardiotoxicity can have a tremendous impact on the type of anticancer therapies that patients can receive as well as the long-term morbidity and mortality outcomes of patients with cancer. Effective management of patients with both cancer and cardiovascular disease is of unique interest to healthcare providers including cardio-oncology professionals.

A goal for cardio-oncology professionals is to allow patients with cancer to receive the best possible cancer treatments while safely minimizing cancer therapy-related cardiotoxicity across the entire continuum of cancer care. Before initiation of cancer therapies with known cardiotoxicity profiles, a cardio-oncology team can identify and treat certain cardiovascular risk factors and pre-existing cardiovascular disease. This team can also define an appropriate prevention and surveillance plan for early identification and appropriate management of potential cardiovascular complications. Implementations described herein can provide information and patient data to help in this regard. In further scenarios, after cancer treatment has been completed, focus shifts to coordination of long-term follow-up and treatment for cardiovascular conditions. For patients on long-term cancer therapies with cardiotoxicity risk, surveillance using the technologies herein can continue until the treatment is finished, a period of time after the treatment is finished, and/or for re-assessment of cardiovascular risks in patients requiring treatment for secondary malignancies.

As discussed in further detail below, the wearable cardiotoxicity monitoring systems implementing the devices, methods, and techniques disclosed herein can aid prescribing physicians and other caretakers in supervising oncology patients while they undergo oncology treatments. More specifically, the wearable cardiotoxicity monitoring systems described herein may help caretakers monitor the cardiotoxicity of oncology patients receiving chemotherapy and/or radiation therapy. Such monitoring may occur before beginning a treatment regimen, during the treatment, and/or after the completion of the treatment regimen, including in scenarios where patients need follow up care and/or ongoing monitoring for recurrence or secondary malignancies. In implementations, at the outset, the system is configured to establish baselines for each cancer patient. The baselines can be based on patient health history, ECG, transthoracic echocardiography, blood and fluid panels, and the like, as laid out in greater detail below.

In implementations, the wearable cardiotoxicity monitoring system includes a wearable cardiotoxicity monitoring device that is configured for long-term, continuous wear by an oncology patient. The wearable cardiotoxicity monitoring device has a number of externally applied biosignal sensors configured to sense one or more biosignals from the patient. These biosignals may include electrical signals indicative of ECG activity, cardiovibrational or bioacoustics signals indicative of cardiovibrational biomarkers of the patient's heart, motion signals or impedance-based transthoracic signals indicative of the patient's respiration rate, radiofrequency (RF) waves that can be processed to generate information about the patient's thoracic fluid level, RF waves and/or photoplethysmography signals that can be processed to generate information about the patient's blood pressure, and/or the like. Thus, in various implementations, the wearable cardiotoxicity monitoring device includes a number of ECG electrodes and at least one non-ECG physiological sensor.

The wearable cardiotoxicity monitoring device also includes a controller configured to transmit biosignal-based data to a remote server. In implementations, the biosignal-based data is the raw or minimally processed biosignals sensed by the externally applied biosignal sensors. Alternatively, or additionally, the biosignal-based data includes data processed and/or analyzed by the wearable cardiotoxicity monitoring device before transmission to the remote server. For example, the wearable cardiotoxicity monitoring device may digitize biosignals, filter biosignals to remove noise, filter biosignals to remove motion artifacts, discard invalid measurements (e.g., measurements taken when the patient was moving above a predetermined threshold, as determined by accelerometer counts), and/or the like. Further, in implementations, the wearable cardiotoxicity monitoring device includes a network interface configured to establish communication sessions directly with the remote server. Alternatively, or additionally, the wearable cardiotoxicity monitoring device may further include an intermediary communications device, such as a portable gateway, configured to relay transmissions between the controller and the remote server.

The wearable cardiotoxicity monitoring system as described herein also includes the remote server, in various implementations. The remote server is in communication with the wearable cardiotoxicity monitoring device and includes a memory implemented in a non-transitory media, as well as at least one processor in communication with the memory. The remote server is configured to receive the biosignal-based data from the wearable cardiotoxicity monitoring device and analyze the biosignal-based data from the wearable cardiotoxicity monitoring device to identify at least one cardiotoxicity biosignal marker associated with heart failure caused by chemotherapy cardiotoxicity and/or radiation therapy cardiotoxicity. Such biosignal markers may include, for example, ECG markers (e.g., PR interval duration, QRS complex duration, QT interval duration, etc.), cardiac acoustic markers (e.g., S1 timings, S2 timings, S3 timings, S4 timings, electomechanical activation times (EMATs), etc.), heart rate, respiration rate, thoracic fluid level, blood pressure, and/or the like.

The remote server may then determine the current status of cardiotoxicity in the patient based on the identified at least one cardiotoxicity biosignal marker. For example, the current status of cardiotoxicity may be a determined risk level for a cardiac condition, such as AV block. As another example, the current status of cardiotoxicity may be an evaluation of the patient's overall cardiac health. Based on the determined current status of cardiotoxicity in the patient, the remote server generates an output. In implementations, determining the current status of cardiotoxicity in the patient may include determining a cardiotoxicity trend over time. As an example, the current trend of cardiotoxicity may be a changing risk level for a cardiac condition, such as AV block. As another example, the current trend of cardiotoxicity may be changes in the patient's overall cardiac health. Based on the determined current trend of cardiotoxicity in the patient, the remote server may generate an output. Such an output may be, for instance, a report sent to the patient's prescribing physician, a flag or other marker regarding the patient shown to the patient's prescribing physician on a monitoring dashboard (e.g., accessible by a computing device used by the prescribing physician), an alert sent to the patient's physician, an alert sent to the patient, and/or the like.

Alternatively, or additionally, the wearable cardiotoxicity monitoring device may perform some or all of the analysis and output generation described above as being performed by the remote server. As an example, the wearable cardiotoxicity monitoring device may identify at least one cardiotoxicity biosignal marker, determine a current status and/or trend of cardiotoxicity in the patient, and generate an output based on the determined current status of cardiotoxicity. The wearable cardiotoxicity monitoring device may then transmit the output to the remote server or to another device, such as a computing device used by the patient's prescribing physician. In some cases, the wearable cardiotoxicity monitoring device may also be configured to provide treatment to the patient and use the output to determine whether to provide the treatment to the patient. For instance, if the output indicates that the patient is experiencing a cardiac arrhythmia that is treatable by a defibrillation shock, the wearable cardiotoxicity monitoring device may be configured to generate and deliver such a shock to the patient.

In one example use case, a physician overseeing an oncology patient during oncology treatment may prescribe that the patient use a wearable cardiotoxicity monitoring device while the patient is receiving their oncology treatment regimen. The wearable cardiotoxicity monitoring device gathers biosignal-based data on the patient and transmits the biosignal-based data to a remote server, which processes the received biosignal-based data to determine the patient's current cardiotoxicity status. The remote server generates hourly, daily, every 36 hours, every 48 hours, weekly, monthly and/or end of use (EOU) reports for the patient's prescribing physician that the physician can access on a monitoring dashboard. In implementations, the remote server can be configured to generate more frequent reports (e.g., hourly or daily reports) for a certain subset of patients decmed high risk (e.g., based on physician input and/or automatically identified by the system as described herein). In implementations, the remote server can be configured to generate the more frequent reports for a period of time (e.g., a few weeks) for a certain subset of patients initially deemed high risk before changing the report frequency to weekly or monthly. The monitoring dashboard presents the weekly report on the patient along with reports on other oncology patients using a wearable cardiotoxicity monitoring device that the physician is also overseeing. The patient's weekly reports includes metrics on the patient's cardiac health with flags on metrics that transgress normal and/or recommended target levels. The flags may be color-coded based on severity. For example, a metric with some degree of cardiotoxicity risk may be coded in yellow, whereas a metric with a high degree of cardiotoxicity risk may be coded in red. If the patient's overall cardiac health deteriorates below a certain level (e.g., a predetermined number or percentage of the patient's metrics are flagged, at least one metric becomes high risk, etc.), the remote server may send the prescribing physician an alert (e.g., to a physician designated personal device). As an illustration, the remote server may send the prescribing physician an email or text message alerting the physician to review the patient's current cardiac health metrics uploaded to the dashboard.

In another example use case, a physician overseeing an oncology patient during oncology treatment may prescribe that the patient use a wearable cardiotoxicity monitoring device before the patient begins the treatment regimen. For example, the patient may wear the wearable cardiotoxicity monitoring device for a one-weck period, two-week period, three-week period, etc. before starting chemotherapy and/or radiation therapy. The remote server may use biosignal-based data gathered and transmitted by the wearable cardiotoxicity monitoring device to prepare baselines for the patient before the patient begins treatment. Such baselines may include, for instance, a baseline ECG for the patient from which the patient's baseline heart rate, the patient's baseline PR interval, the patient's baseline QRS complex duration, the patient's baseline QT interval, and/or the like can be derived. In some examples, the baselines can also include the patient's baseline thoracic fluid level (e.g., thoracic fluid content (TFC) level) based on, for instance, RF base measurement devices or sensors, as discussed in further detail below.

In implementations, the baseline information that is generated or retrieved for the patient at the outset also includes clinical assessment information. For example, such clinical assessment information may include the patient's cancer treatment history, cardiovascular history, cardiovascular risk factors, findings from an initial physical examination of the patient, and vital signs measurements (e.g., resting heart rate, blood pressure, weight, height, gender, and body mass index (BMI) information). In implementations, the baseline information can also include results from cardiotoxicity blood and fluids panels, including cardiac biomarkers such as troponin (cTn) and natriuretic peptides (NP) (including B-type natriuretic peptides that are measured in patients at risk of cardiac dysfunction based on the patient's clinical status), type of cancer treatment, and kidney function. Additional baseline information can include fasting plasma glucose, HbAlc, kidney function information, estimated glomerular filtration rate (eGFR), a lipid profile, and in some cases, transthoracic echocardiography.

In some cases, the remote server may determine whether the patient's heart is healthy enough for the patient to begin the chemotherapy and/or radiation therapy treatment. To illustrate, if the remote server finds that the patient already is experiencing AV block, has a ventricular ejection fraction below a predetermined threshold, has a predetermined thoracic fluid level, etc., the remote server may recommend to the physician that the physician alter the planned treatment. These recommendations may include suggesting a different type of chemotherapy, suggesting radiation therapy instead of chemotherapy, recommending that the patient use a wearable cardiotoxicity monitoring device during treatment, recommending that the patient use a wearable cardiotoxicity monitoring device that can also provide defibrillation therapy to the patient, and/or the like.

In another example use case, a physician overseeing an oncology patient may prescribe that the patient use a wearable cardiotoxicity monitoring device after the patient finishes an oncology treatment. The patient may wear the wearable cardiotoxicity monitoring device for several weeks to several months to several years. Using biosignal-based data from the wearable cardiotoxicity monitoring device, the remote server may be able to determine the current status of the patient's cardiac health, for example, compared to a baseline taken before the patient began treatment. In implementations, the patient may use the wearable monitoring device long enough for the remote server to determine a current level of cardiotoxicity in the patient. In implementations, the patient may use the wearable cardiotoxicity monitoring device over time such that the physician can monitor lasting side effects of the patient's oncology treatment.

The wearable cardiotoxicity monitoring systems described herein may provide advantages over prior art systems. For example, patients receiving chemotherapy and/or radiation therapy treatments may need cardiotoxicity monitoring during treatment such that their prescribing physician or other caregiver can determine whether the treatments are having undue effects on their heart. Currently, the patient's heart health may be evaluated during appointments with their prescribing physician or other caregiver. However, these appointments may be on a periodic basis, which does not give their prescribing physician or other caregiver continual insight into their cardiac health. Moreover, if the patient's cardiac health takes a drastic downturn, such that the patient's chemotherapeutic and/or radiation therapy treatments should be immediately modified or stopped, this change in cardiac health will not be caught by the prescribing physician or other caregiver until the next appointment. By contrast, the wearable cardiotoxicity monitoring systems described herein allow for continual monitoring of the patient's cardiac health during the prescribed wear period. Additionally, the wearable cardiotoxicity monitoring systems can alert the patient's prescribing physician or another caregiver, or even the patient themselves, if the patient reaches a level of cardiotoxicity that should be immediately treated. In some cases, the wearable cardiotoxicity monitoring systems may also be used to determine a baseline for the patient's cardiac health that subsequent measurements can be compared to, and/or whether the patient's heart is healthy enough for a chemotherapy and/or radiation therapy treatment. Similarly, in some cases, the patient may use the wearable cardiotoxicity monitoring systems after the patient's treatment regimen has finished to determine post-treatment cardiac effects that may need follow-up treatments. For example, the patient may use the wearable cardiotoxicity monitoring systems described herein for an extended period after completion of their chemotherapy and/or radiation therapy treatments such that their prescribing physician or other caregiver (e.g., a cardiologist) can evaluate their post-treatment cardiac status.

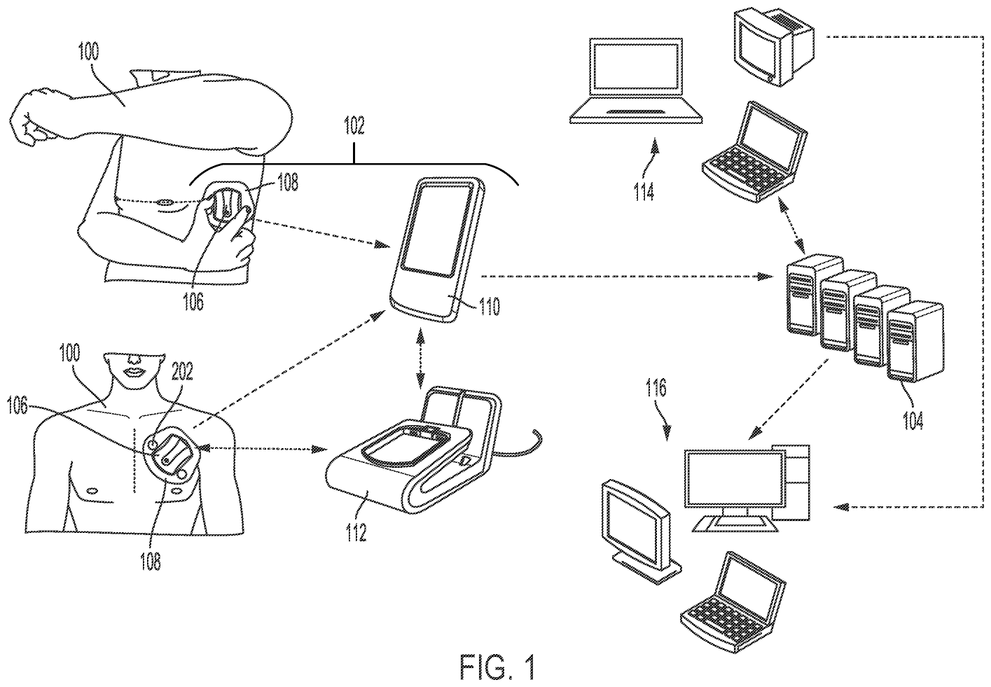

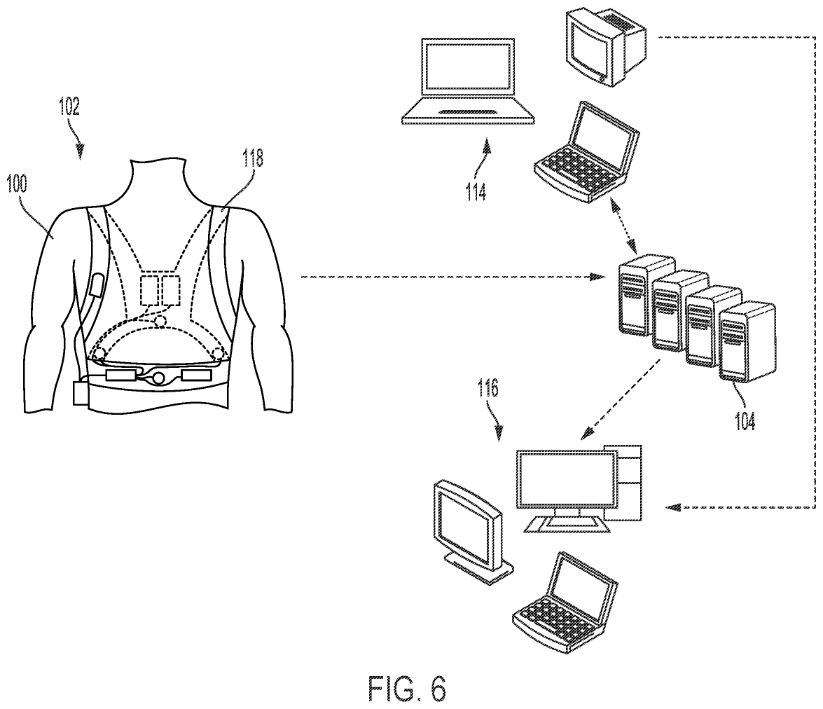

FIG. 1 illustrates an example of a wearable cardiotoxicity monitoring system for monitoring cardiotoxicity biosignal markers in a patient 100, according to implementations disclosed herein. The wearable cardiotoxicity monitoring system of FIG. 1 includes a wearable cardiotoxicity monitoring device 102 configured for long-term continuous wear by the patient 100, where the wearable cardiotoxicity monitoring device 102 is in communication with a remote server 104. The wearable cardiotoxicity monitoring device 102 includes a number of externally applied biosignal sensors configured to sense one or more biosignals from the patient 100, as described in further detail below. In the example of FIG. 1, the wearable cardiotoxicity monitoring device 102 includes a cardiotoxicity monitoring unit 106 coupled to a removable adhesive patch 108. However, other implementations of the wearable cardiotoxicity monitoring system may include different embodiments of a wearable cardiotoxicity monitoring device 102 (e.g., as described with reference to FIGS. 6-8, 19, and 20 below).

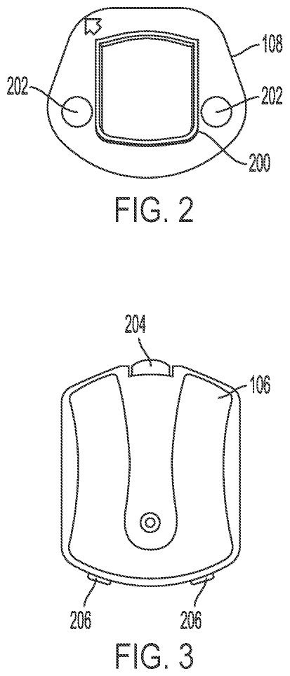

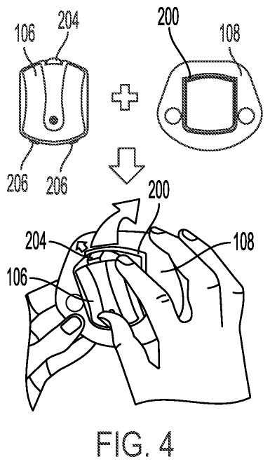

The adhesive patch 108 is configured to be adhesively attached or coupled to the skin of the patient 100. The adhesive patch 108 is further configured such that the cardiotoxicity monitoring unit 106 may be removably attached to the adhesive patch 108. For example, referring to FIG. 2, the adhesive patch 108 may include a frame 200 (e.g., a plastic frame) delineating the boundary of the region of the adhesive patch 108 that is configured for housing the cardiotoxicity monitoring unit 106. The adhesive patch 108 may be disposable (e.g., single-, few-, or multiple-use patches) and may be made of biocompatible, non-woven material. In some embodiments, the cardiotoxicity monitoring unit 106 may be designed for long-term, continuous usage. In such embodiments, the connection between the cardiotoxicity monitoring unit 106 and the adhesive patch 108 may be configured to be reversible. For example, the cardiotoxicity monitoring unit 106 may be configured to be removably attached to the adhesive patch 108. In implementations, as shown in FIG. 3, the cardiotoxicity monitoring unit 106 may include components such as a snap-in clip 204 that is configured to secure the cardiotoxicity monitoring unit 106 to the adhesive patch 108 upon attachment to the patch frame 200. After the cardiotoxicity monitoring unit 106 is attached to the frame 200, a user may press the snap-in clip 204 to subsequently release the cardiotoxicity monitoring unit 106 from the frame 200. In implementations, as further shown in FIG. 3, the cardiotoxicity monitoring unit 106 may include positioning tabs 206 that facilitate the attachment process between the cardiotoxicity monitoring unit 106 and the adhesive patch 108. For example, the positioning tabs 206 may guide a user to insert the cardiotoxicity monitoring unit 106 onto the correct portion of the frame 200 such that the cardiotoxicity monitoring unit 106 can then be coupled, connected, or snapped into the frame 200 using the snap-in clip 204, as shown in FIG. 4.

Referring back to FIG. 1, FIG. 1 illustrates example locations on the body of the patient 100 where an adhesive patch 108 housing the cardiotoxicity monitoring unit 106 may be placed. As an example, the adhesive patch 108 may be placed on the side of the patient's thorax (e.g., under the patient's armpit, level with a circumferential line running across the patient's chest). As another example, the adhesive patch 108 may be placed on the front of the patient's thorax (e.g., to the left of a medial line running between the patient's collarbone, for patients without dextrocardia). However, the adhesive patch 108 may also be placed on any part of the patient's thorax that allows for efficient monitoring and recording of biosignals from the patient 100. For instance, the adhesive patch 108 may be placed over the patient's sternum, over a lower portion of the patient's front or side thorax (e.g., under the patient's sternum), and/or the like.

The cardiotoxicity monitoring unit 106 and adhesive patch 108 are configured for long-term and/or extended use or wear by, or attachment or connection to, the patient 100. For example, devices as described herein are capable of being continuously used or continuously worn by, or attached or connected to, the patient 100 without substantial interruption (e.g., for 24 hours, 2 days, 5 days, 7 days, 2 weeks, 30 days or 1 month, or beyond such as multiple months or even years). In some implementations, such devices may be removed for a period of time before use, wear, attachment, or connection to the patient 100 is resumed. As an illustration, the cardiotoxicity monitoring unit 106 may be removed for charging, to carry out technical service, to update the device software or firmware, for the patient 100 to take a shower, and/or for other reasons or activities without departing from the scope of the examples described herein. As another illustration, the patient 100 may remove a used adhesive patch 108, as well as the cardiotoxicity monitoring unit 106, so that the patient 100 may adhere a new adhesive patch 108 to their body and attach the cardiotoxicity monitoring unit 106 to a new adhesive patch 108. Such substantially or nearly continuous use, monitoring, or wear as described herein may nonetheless be considered continuous use, monitoring, or wear.

For example, in implementations, the adhesive patch 108 may be designed to maintain attachment to skin of the patient 100 for several days (e.g., in a range from about 4 days to about 10 days, from about 3 days to about 5 days, from about 5 days to about 7 days, from about 7 days to about 10 days, from about 10 days to about 14 days, from about 14 days to about 30 days, etc.). After the period of use, the adhesive patch 108 can be removed from the patient's skin and the cardiotoxicity monitoring unit 106 can be removed from the adhesive patch 108. The cardiotoxicity monitoring unit 106 can then be removably coupled, connected, or snapped onto a new adhesive patch 108 and reapplied to the patient's skin.

As further shown in FIG. 1, the wearable cardiotoxicity monitoring device 102 may include a portable gateway 110 configured to facilitate communication between the wearable cardiotoxicity monitoring device 102 and the remote server 104. In implementations, the portable gateway 110 is configured to receive data and signals provided by the wearable cardiotoxicity monitoring device 102 (e.g., biosignal-based data based on one or more biosignals sensed by the wearable cardiotoxicity monitoring device 102) and transmit the data and signals to the remote server 104. As an illustration, in implementations, the portable gateway 110 may communicate with the remote server 104 via Ethernet, via Wi-Fi, via near-field communication (NFC), via radiofrequency, via cellular networks, via Bluetooth®-to-TCP/IP access point communication, and/or the like. Accordingly, the portable gateway 110 may include communications circuitry configured to implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In implementations, the wearable cardiotoxicity monitoring device 102 and/or the portable gateway 110 may include communications circuitry that is part of an Internet of Things (IoT) and communicate with each other and/or the remote server 104 via IoT protocols (e.g., Constrained Application Protocol (CoAP), Message Queuing Telemetry Transport (MQTT), Wi-Fi, Zigbee, Bluetooth®, Extensible Messaging and Presence Protocol (XMPP), Data-Distribution Service (DDS), Advanced Messaging Queuing Protocol (AMQP), and/or Lightweight M2M (LwM2M)).

Alternatively, in other implementations, the wearable cardiotoxicity monitoring device 102 may be configured to transmit data and/or signals directly to the remote server 104 instead of, or in addition to, transmitting the signals to the portable gateway 110. Accordingly, the wearable cardiotoxicity monitoring device 102 may be in wired or wireless communication with the remote server 104. As an illustration, the wearable cardiotoxicity monitoring device 102 may communicate with the remote server 104 via Ethernet, via Wi-Fi, via near-field communication (NFC), via radiofrequency, via cellular networks, via Bluetooth®-to-TCP/IP access point communication, and/or the like. Further, in some implementations, the wearable cardiotoxicity monitoring system may not include the portable gateway 110. In such implementations, the wearable cardiotoxicity monitoring device 102 may perform the functions of the portable gateway 110 described herein. Additionally, in implementations where the wearable cardiotoxicity monitoring device 102 is configured to communicate directly with the remote server 104, the wearable cardiotoxicity monitoring device 102 may include communications circuitry configured to implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In implementations, as indicated above, the communications circuitry in the wearable cardiotoxicity monitoring device 102 may be part of an IoT and communicate with the remote server 104 via IoT protocols for handling secure (e.g., encrypted) messaging and routing.

The wearable cardiotoxicity monitoring system may also include a charger 112, as further shown in FIG. 1. In implementations, the charger 112 includes charging cradles configured to hold and recharge the cardiotoxicity monitoring unit 106 and the portable gateway 110. Alternatively, in some implementations, the wearable cardiotoxicity monitoring system may not include the portable gateway 110, and accordingly, the charger 112 may be configured to hold the cardiotoxicity monitoring unit 106 alone. In implementations, the cardiotoxicity monitoring unit 106 and the portable gateway 110 may have separate chargers, or one or both of the cardiotoxicity monitoring unit 106 and the portable gateway 110 may have removable batteries that may be replaced and/or recharged.

The remote server 104 is configured to receive and process the data and/or signals received from the wearable cardiotoxicity monitoring device 102. In implementations, the remote server 104 may be in electronic communication with a number of wearable cardiotoxicity monitoring devices 102 and be configured to receive and process the data and/or signals received from all of the wearable cardiotoxicity monitoring devices 102 in communication with the remote server 104. The remote server 104 may include a computing device, or a network of computing devices, including at least one database (e.g., implemented in non-transitory media or memory) and at least one processor configured to execute sequences of instructions (e.g., stored in the database, with the at least one processor being in communication with the database) to receive and process the data and/or signals received from the wearable cardiotoxicity monitoring device 102. For example, the at least one processor of the remote server 104 may be implemented as a digital signal processor (DSP), such as a 24-bit DSP processor; as a multicore-processor (e.g., having two or more processing cores); as an Advanced RISC Machine (ARM) processor, such as a 32-bit ARM processor; and/or the like. The at least one processor of the remote server 104 can execute an embedded operating system and further execute services provided by the operating system, where these services can be used for file system manipulation, display and audio generation, basic networking, firewalling, data encryption, communications, and/or the like. The database may be implemented as flash memory, solid state memory, magnetic memory, optical memory, cache memory, combinations thereof, and/or others. In various implementations, the remote server 104 may use the data received from the wearable cardiotoxicity monitoring device 102 to determine a current status of cardiotoxicity in the patient 100, as described in further detail below. Alternatively, in some implementations, the wearable cardiotoxicity monitoring device 102 may perform some or all of the analysis described herein as being performed by the remote server 104 (e.g., with the wearable cardiotoxicity monitoring device 102 transmitting the current status of cardiotoxicity in the patient 100 or another output to the remote server 104, for instance, via the portable gateway 110).

As shown in FIG. 1, in implementations, the wearable cardiotoxicity monitoring system further includes one or more user interfaces, such as technician interfaces 114 and caregiver interfaces 116. The technician interfaces 114 and caregiver interfaces 116 are in electronic communication with the remote server 104 through a wired or wireless connection. For instance, the technician interfaces 114 and caregiver interfaces 116 may communicate with the remote server 104 via Wi-Fi, via Ethernet, via cellular networks, and/or the like. Additionally, as shown, at least some of the technician interfaces 114 may be in electronic communication with at least some of the caregiver interfaces 116 through a wired or wireless connection, such as via Wi-Fi, via Ethernet, via cellular networks, and/or the like. The technician interfaces 114 and the caregiver interfaces 116 may include, for example, desktop computers, laptop computers, and/or portable personal digital assistants (e.g., smartphones, tablet computers, etc.).

In implementations, the technician interfaces 114 are configured to electronically communicate with the remote server 104 for the purpose of viewing and analyzing information gathered from one or more wearable cardiotoxicity monitoring devices 102 (e.g., biosignal-based data received from a wearable cardiotoxicity monitoring device 102, one or more cardiotoxicity biosignal markers identified from the biosignal-based data, a current status of cardiotoxicity in the patient 100, and/or an output based on the current status of cardiotoxicity in the patient 100, as described in further detail below). For example, a technician interface 114 may provide one or more instructions to the remote server 104 to prepare a report on data and/or signals received from a given wearable cardiotoxicity monitoring device 102 for a certain time period. Accordingly, a technician interface 114 may include a computing device having a processor communicably connected to a memory and a visual display. The technician interface 114 may display to a user of the technician interface 114 (e.g., a technician) data received from the one or more wearable cardiotoxicity monitoring devices 102 and/or information computed from the data and/or signals received from the one or more wearable cardiotoxicity monitoring devices 102, described in further detail below. The user of the technician interface 114 may then provide one or more inputs to the remote server 104 to guide the remote server 104 in preparing a report for a patient 100.

As an example, a user of a technician interface 114 may select a time period to use for the report, and the remote server 104 may prepare a report corresponding to the selected time period. As another example, a user of a technician interface 114 may select types of data to be included in a report, such as a cardiotoxicity biomarker signal associated with heart failure caused by chemotherapy cardiotoxicity and/or radiation therapy cardiotoxicity, as described in more detail below. The remote server 104 may then prepare a report according to the types of data selected by the user. As another example, a user of a technician interface 114 may view a report prepared by the remote server 104 and draft a summary of the report to be included in a summary section for the report. Alternatively, in implementations, the remote server 104 may analyze, summarize, etc. the data and/or signals received from the one or more wearable cardiotoxicity monitoring devices 102 with minimal or no input or interaction with a technician interface 114. In this way, the remote server 104 may analyze, summarize, etc. the information gathered from the one or more wearable cardiotoxicity monitoring devices 102 and prepare a report on this information through a completely or mostly automated process.

The caregiver interfaces 116 are configured to electronically communicate with the remote server 104 for the purpose of viewing information on various patients 100 using a wearable cardiotoxicity monitoring device 102. As such, a caregiver interface 116 may include a computing device having a processor communicably connected to a memory and a visual display. The caregiver interface 116 may display to a user of the caregiver interface 116 (e.g., a physician, a nurse, or other caregiver) biosignal-based data received from a wearable cardiotoxicity monitoring device 102, one or more cardiotoxicity biosignal markers identifies from the biosignal-based data, a current status of cardiotoxicity in the patient 100, an output based on the current status of cardiotoxicity in the patient 100, and/or the like, as described in further detail below. In implementations, the caregiver interface 116 may display to a user one or more reports summarizing the biosignal-based data received from a wearable cardiotoxicity monitoring device 102, one or more cardiotoxicity biosignal markers identified from the biosignal-based data, a current status of cardiotoxicity in the patient 100, an output based on the current status of cardiotoxicity in the patient 100, and/or the like. For example, the caregiver interface 116 may display reports prepared by the remote server 104 (e.g., based on inputs from one or more technician interfaces 114). In implementations, the user of a caregiver interface 116 may be able to interact with the information displayed on the caregiver interface 116. As an example, the user of a caregiver interface 116 may be able to select a portion of a patient report and, in response, be able to view additional information relating to the selected portion of the report. Additional information may include, for instance, the biosignal-based data received from the wearable cardiotoxicity monitoring device 102 used to prepare the report, one or more cardiotoxicity biosignal markers used to prepare the report, and/or the like. In implementations, the user of the caregiver interface 116 may instead view a static patient report that does not have interactive features.

In implementations, a technician interface 114 and/or a caregiver interface 116 may be a specialized interface configured to communicate with the remote server 104. As an example, the technician interface 114 may be a specialized computing device configured to receive preliminary patient reports from the remote server 104, receive inputs from a user to adjust the preliminary report, and transmit the inputs back to the remote server 104. The remote server 104 then uses the inputs from the technician interface 114 to prepare a finalized patient report, which the remote server 104 also transits to the technician interface 114 for review by the user. As another example, the caregiver interface 116 may be a specialized computing device configured to communicate with the remote server 104 to receive and display patient reports, as well as other information regarding patients 100 using a wearable cardiotoxicity monitoring device 102.

In implementations, a technician interface 114 and/or a caregiver interface 116 may be a generalized user interface that has been adapted to communicate with the remote server 104. To illustrate, the technician interface 114 may be a computing device (e.g., a laptop, a portable personal digital assistant such as a smartphone or tablet, etc.) executing a technician application that configures the computing device to communicate with the remote server 104. For example, the technician application may be downloaded from an application store or otherwise installed on the computing device. Accordingly, when the computing device executes the technician application, the computing device is configured to establish an electronic communication link with the remote server 104 to receive and transmit information regarding patients 100 using a wearable cardiotoxicity monitoring device 102. Similarly, the caregiver interface 116 may be a computing device (e.g., a laptop, a portable personal digital assistant such as a smartphone or tablet, etc.) executing a caregiver application that configures the computing device to communicate with the remote server 104. The caregiver application may be similarly downloaded from an application store or otherwise installed on the computing device and, when executed, may configure the computing device to establish a communication link with the remote server 104 to receive and display information on patients 100 using a wearable cardiotoxicity monitoring device 102.

The application store is typically included within an operating system of a computing device implementing a user interface. For example, in a device implementing an operating system provided by Apple Inc. (Cupertino, California), the application store can be the App Store, a digital distribution platform, developed and maintained by Apple Inc., for mobile apps on its iOS and iPadOS® operating systems. The application store allows a user to browse and download an application, such as the technician or caregiver application, developed in accordance with the Apple® iOS Software Development Kit. For instance, such technician or caregiver application may be downloaded on an iPhone® smartphone, an iPod Touch® handheld computer, or an iPad® tablet computer, or transferred to an Apple Watch® smartwatch. Other application stores may alternatively be used for other types of computing devices, such as computing devices operating on the Android® operating system.

In some implementations, the technician application and the caregiver application may be the same application, and the application may provide different functionalities to the computing device executing the application based on, for example, credentials provided by the user. For instance, the application may provide technician functionalities to a first computing device in response to authenticating technician credentials entered on the first computing device, and may provide caregiver functionalities to a second computing device in response to authenticating caregiver credentials entered on the second computing device. In other cases, the technician application and the caregiver application may be separate applications, each providing separate functionalities to a computing device executing them.

In implementations, the wearable cardiotoxicity monitoring system shown in FIG. 1 may include other types of interfaces. To illustrate, in examples, the wearable cardiotoxicity monitoring system may include patient interfaces. The remote server 104 and/or a technician interface 114 may provide a report on a patient 100 using a wearable cardiotoxicity monitoring device 102 to this patient 100 via a patient interface. This patient report may be the same as a report provided to a caregiver via a caregiver interface 116, or this report may be different from the report provided to the caregiver via the caregiver interface 116. For instance, the report provided to a patient 100 may be an abridged version of a report prepared for the patient's caregiver, such as a report that summarizes the key information from the report prepared for the patient's caregiver. In various implementations, the patient interface may be configured similarly to and function similarly to the caregiver interface 116 discussed above. As an example, the patient interface may be similar to the caregiver interface 116 but with some additional restrictions on what is included in a patient report compared to a report prepared for the patient's caregiver and/or on the functionalities of, for instance, an application executed by the patient interface that the patient 100 can use to access and view a patient report or other information on the patient 100.

Returning back to the wearable cardiotoxicity monitoring device 102, as noted above the wearable cardiotoxicity monitoring device 102 includes a number of externally applied biosignal sensors configured to sense one or more biosignals from a patient. To illustrate, in embodiments of the wearable cardiotoxicity monitoring device 102 that include a cardiotoxicity monitoring unit 106 and an adhesive patch 108, as described above with reference to FIG. 1, the externally applied biosignal sensors may be implemented in the cardiotoxicity monitoring unit 106 and/or implemented in the adhesive patch 108. For example, at least a portion of the externally applied biosignal sensors may be integrated in the adhesive patch 108. As another example, at least a portion of the externally applied biosignal sensors may be configured to be removably mounted onto the adhesive patch 108 (e.g., through being integrated in or otherwise implemented via the cardiotoxicity monitoring unit 106).

In implementations, a number of ECG electrodes 202 may be embedded into the adhesive patch 108, as shown in FIG. 2. The ECG electrodes 202 are configured to sense one or more electrical signals indicative of ECG activity from a skin surface of the patient 100 (e.g., skin contacted directly or through a covering). The ECG electrodes 202 may be coupled to the cardiotoxicity monitoring unit 106 by dedicated wiring within the adhesive patch 108, where the cardiotoxicity monitoring unit 106 receives electrical signals sensed by the ECG electrodes 202 via the dedicated metal contacts or traces on the cardiotoxicity monitoring unit 106 when the cardiotoxicity monitoring unit 106 is mounted on the adhesive patch 108. Example ECG electrodes 202 include a metal electrode with an oxide coating, such as tantalum pentoxide electrodes. For instance, digital sensing electrodes can include skin-contacting electrode surfaces that may be deemed polarizable or non-polarizable depending on a variety of factors, including the metals and/or coatings used in constructing the electrode surface. All such electrodes can be used with the principles, techniques, devices, and systems described herein. As an illustration, the electrode surfaces can be based on stainless steel, noble metals such as platinum, or Ag—AgCl.

In examples, the ECG electrodes 202 can be used with an electrolytic gel dispersed between the electrode surface and the patient's skin. In other examples, the ECG electrodes 202 can be dry electrodes that do not need an electrolytic material or optionally can be used with an electrolytic material. For instances, such a dry electrode can be based on tantalum metal and have a tantalum pentoxide coating, as is described above. Such dry electrodes may be more comfortable for long-term monitoring applications, in various implementations.

In implementations, the ECG electrodes 202 can include additional components such as accelerometers, acoustic signal detecting devices, cardiovibrational sensors, or other measuring devices for additional parameters. For example, the ECG electrodes 202 may be configured to detect other types of patient biosignals or other physiological signals, such as thoracic fluid levels, heart vibrations, lung vibrations, respiration vibrations, patient movement, etc. Alternatively, or additionally, the cardiotoxicity monitoring unit 106 and/or the adhesive patch 108 may include sensors or detectors separate from the ECG electrodes 202, such as separate motion detectors, bioacoustics sensors, cardiovibrational sensors, respiration sensors, temperature sensors, pressure sensors, and/or the like, as described in further detail below.

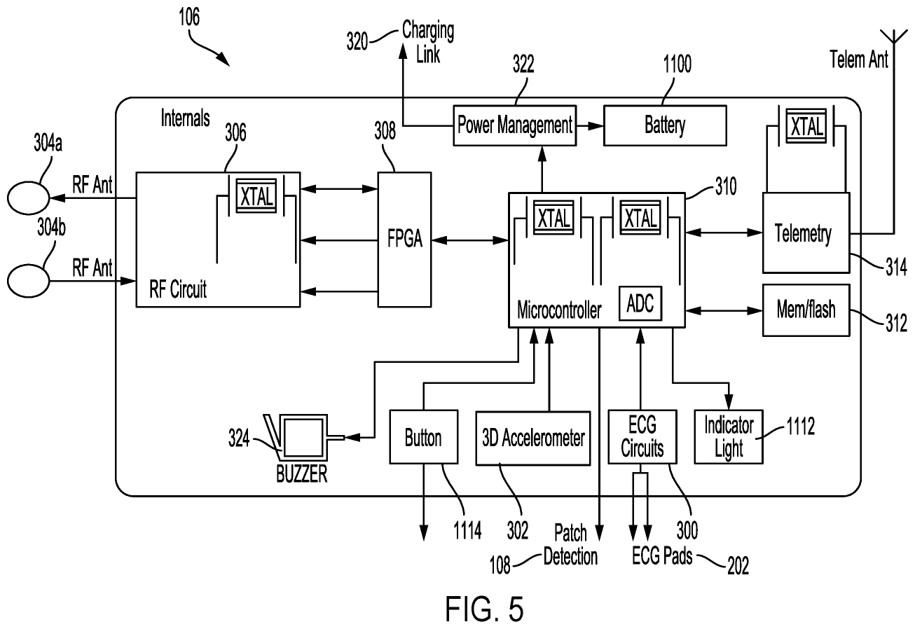

FIG. 5 illustrates an example electronic architecture for the cardiotoxicity monitoring unit 106. The ECG circuits 300 may receive signals from the ECG electrodes 202 when the cardiotoxicity monitoring unit 106 is attached to the adhesive patch 108, where the signals received from the ECG electrodes 202 include ECG waveforms sensed from the patient 100. In embodiments, the ECG electrodes 202 and cardiotoxicity monitoring unit 106 may have a sampling rate in a range from about 250 Hz to about 500 Hz, from about 300 Hz to about 450 Hz, from about 350 Hz to about 400 Hz, including values and subranges therebetween. In embodiments, the cardiotoxicity monitoring unit 106 may sample the signals from the ECG electrodes 202 after applying band-pass filtering by a 12 bit ADC. In implementations, during normal operation, the cardiotoxicity monitoring unit 106 may transfer data from the ECG electrodes 202 to the remote server 104 “as-is,” where the remote server 104 uses the data for analysis as described in further detail below. Alternatively, in other implementations, the cardiotoxicity monitoring unit 106 may perform at least some processing of the data from the ECG electrodes 202 (e.g., filtering of the data, analysis of the data, etc.). For example, the cardiotoxicity monitoring unit 106 may generated digitized ECG signals from the data received from the ECG electrodes 202 and transmit the digitized ECG signals to the remote server 104 (e.g., directly or via the portable gateway 110).

In implementations, the wearable cardiotoxicity monitoring device 102 can be designed to include a digital front-end where analog signals sensed by skin-contacting electrode surfaces of a set of digital sensing electrodes are converted to digital signals for processing. Wearable, ambulatory medical devices with analog front-end configurations may use circuitry to accommodate a signal with a high source impedance acquired via a sensing electrode (e.g., having an internal impedance range from approximately 100 kiloohms to one or more megaohms). This high source impedance signal is processed and transmitted to a monitoring device (e.g., the microcontroller 310 discussed below) for further processing. In certain implementations, the monitoring device, or another similar processor such as a microprocessor or another dedicated processor operably coupled to the sensing electrodes, can be configured to receive a common noise signal from each of the sensing electrodes, sum the common noise signals, invert the summed common noise signals, and feed the inverted signal back into the patient 100 as a driven ground using, for example, a driven right leg circuit to cancel out common mode signals.

In addition to ECG electrodes 202, the cardiotoxicity monitoring unit 106 may also include and/or be connected to one or more non-ECG physiological sensors configured to sense non-ECG biosignals from the patient 100, according to various implementations. To illustrate, the cardiotoxicity monitoring unit 106 may include or be connected to a cardiovibration sensor configured to sense cardiovibrations of the patient 100, a respiration sensor configured to sense respirations of the patient 100, a thoracic fluid sensor configured to sense thoracic fluid levels in the patient 100, a blood pressure sensor configured to sense a blood pressure of the patient 100, and/or the like. Other examples of externally applied sensors configured to sense biosignals may include a P-wave sensor (e.g., a sensor configured to monitor and isolate P-waves within an ECG waveform), an oxygen saturation sensor (e.g., implemented through photoplethysmography, such as through light sources and light sensors configured to transmit light into the patient's body and receive transmitted and/or reflected light containing information about the patient's oxygen saturation), and so on.

For example, as shown in FIG. 5, the cardiotoxicity monitoring unit 106 may include one or more motion sensors such as a 3D accelerometer 302 with three axes and a span of +2 g. Using the 3D accelerometer 302, the cardiotoxicity monitoring unit 106 may acquire data on cardiovibrations of the patient's heart (e.g., vibrations corresponding to the opening and closing of the patient's heart valves) and/or data on the patient's respirations (e.g., data on the patient's respiration rate). In implementations, the cardiotoxicity monitoring unit 106 may include a different type of motion sensor, such as a 1-axis channel accelerometer, 2-axis channel accelerometer, gyroscope, magnetometer, or ballistocardiography sensor.

As another example, the cardiotoxicity monitoring unit 106 may include an RF sensor configured to take bio-impedance measurements of the patient's thorax. In implementations, the cardiotoxicity monitoring unit 106 may then transmit the bio-impedance measurements to the remote server 104, which uses the bio-impedance measurements to determine a thoracic fluid level in the patient 100. In implementations, the cardiotoxicity monitoring unit 106 may determine a thoracic fluid level in the patient 100 from the bio-impedance measurements and transmit the thoracic fluid level to the remote server 104. Accordingly, as shown in FIG. 5, an example embodiment of the cardiotoxicity monitoring unit 106 includes at least one RF antenna, such as a transmitting RF antenna 304a and a receiving RF antenna 304b (or a single antenna configured to transmit and receive RF waves, in other implementations), and RF circuitry 306 configured to transmit a low-power signal in an ultra-high frequency band (e.g., 0.1 GHz to 5.0 GHz, 0.5 GHZ to 2.1 GHZ, etc.) at a predetermined rate (e.g., every 10 ms, every 20 ms, every 30 ms, every 40 ms, every 50 ms, etc.). The cardiotoxicity monitoring unit 106 may also include other circuits for controlling the RF circuitry 306 (e.g., field-programmable gate array (FPGA) circuits 308). The at least one RF antenna 304a, 304b and RF circuitry 306 receive RF-based biosignals indicative of the thoracic fluid level in the patient 100 in the form of RF waves transmitted through the patient 100 and/or scattered or reflected from the patient 100. For example, the at least one RF antenna 304a, 304b and RF circuitry 306 may detect transmitted, scattered, and/or reflected RF waves for a predetermined amount of time (e.g., about 30 seconds, about 1 minute, about 2 minutes, about 3 minutes, about 5 minutes, about 10 minutes, etc.). In implementations, the wearable cardiotoxicity monitoring device 102 (e.g., at the cardiotoxicity monitoring unit 106 and/or at the portable gateway 110) and/or the remote server 104 are configured to gate when RF measurements are taken and/or discard certain RF measurements based on the patient's state when the RF measurements were taken. For example, the wearable cardiotoxicity monitoring device 102 and/or the remote server 104 may determine whether the patient 100 showed movement above a predetermined threshold before the wearable cardiotoxicity monitoring device 102 started the RF measurements process and/or while the RF measurements were taking place.

In implementations, the wearable cardiotoxicity monitoring device 102 may include and/or be connected to other types of non-ECG physiological sensors from the sensors shown in FIG. 5, in addition to the sensors discussed above and/or in the alternate to the sensors discussed above. For example, the wearable cardiotoxicity monitoring device 102 may include and/or be connected to a bioacoustics sensor, such as a microphone, configured to detect cardiovibrations and/or cardiac acoustics from the patient 100 (e.g., instead of an accelerometer, as discussed above). In implementations, the wearable cardiotoxicity monitoring device 102 may include and/or be connected to an impedance-based transthoracic sensor configured to sense transthoracic signals indicative of respiration of the patient 100 (e.g., instead of an accelerometer, as discussed above).

In implementations, the wearable cardiotoxicity monitoring device 102 may include and/or be connected to a blood pressure sensor, such as a blood pressure sensor implemented through an RF sensor (e.g., including the at least one RF antenna 304a, 304b and RF circuitry 306 discussed above) combined with a photoplethysmography (PPG) sensor (e.g., including one or more light emitting diodes (LEDs)). For example, the wearable cardiotoxicity monitoring device 102 may usc the RF sensor to generate information about an aortic waveform (e.g., a waveform of aortic volume over time) and the PPG sensor to generate information about an arterial waveform for arteries a known distance from the aorta. As an illustration, the RF sensor may be provided in the cardiotoxicity monitoring unit 106 as discussed above, where the cardiotoxicity monitoring unit 106 and the adhesive patch 108 are located over the patient's sternum. The cardiotoxicity monitoring unit 106 may transmit RF waves into the patient's thorax and receive reflected and/or scattered RF waves, which the wearable cardiotoxicity monitoring device 102 uses to generate information about the patient's aortic waveform (e.g., at the cardiotoxicity monitoring unit 106 and/or the portable gateway 110). The PPG sensor may be provided in the cardiotoxicity monitoring unit 106 and/or in the adhesive patch 108, and similarly the PPG sensor may be configured to transmit light waves into and receive scattered and/or reflected light waves from surface arteries over the patient's sternum. The wearable cardiotoxicity monitoring device 102 then uses the received light waves to generate information about the patient's arterial waveform (e.g., at the cardiotoxicity monitoring unit 106 and/or the portable gateway 110). As another illustration, instead of the cardiotoxicity monitoring unit 106 and/or adhesive patch 108 including the PPG sensor, the PPG sensor may be provided in another device that is configured with the PPG sensor, such as a wristband or armband. The device with the PPG sensor may be electronically and/or communicably coupled to the wearable cardiotoxicity monitoring device 102 and/or to the portable gateway 110.

Once the information about the aortic waveform and the arterial waveform has been generated, the wearable cardiotoxicity monitoring device 102 may determine the patient's pulse wave velocity by identifying a fiducial point on the aortic waveform (e.g., a point of highest volume or a point of onset in the aorta in a cardiac cycle), identifying a corresponding fiducial point on the arterial waveform (e.g., a point of highest volume or a point of onset in the surface arteries in the same cardiac cycle), determine a time difference between the two fiducial points, and divide the known distance between the aorta and the surface arteries being measured by the time difference between the two fiducial points. The wearable cardiotoxicity monitoring device 102 may then determine the patient's blood pressure from the pulse wave velocity. Alternatively, the wearable cardiotoxicity monitoring device 102 may transmit the information about the patient's aortic waveform and the information about the patient's arterial waveform to the remote server 104. The remote server 104 may then perform some or all of this analysis. Additional details on implementations of a blood pressure sensor as described above may be found in U.S. patent application Ser. No. 17/656,480, filed on Mar. 25, 2022, titled “SYSTEM FOR USING RADIOFREQUENCY AND LIGHT TO DETERMINE PULSE WAVE VELOCITY,” which is hereby incorporated by reference.

In implementations, the wearable cardiotoxicity monitoring device 102 may include and/or be connected to at least one externally applied sensor configured to sense additional types of signals associated with the patient 100. These additional types of signals may be non-biosignals associated with the patient 100. For example, the wearable cardiotoxicity monitoring device 102 may include the 3D accelerometer 302, which is configured to sense motion signals associated with the patient 100 and the patient's movement. The motion signals may include information about the patient's activity level and/or body position, as described in further detail below. Other examples of sensors configured to sense additional types of signals may include temperature sensors, pressure sensors, humidity sensors, and/or the like.

In implementations, the distribution of the externally-applied biosignal sensors of the wearable cardiotoxicity monitoring device 102 may be different from the examples discussed above. For instance, the ECG pads 202 may be provided on the cardiotoxicity monitoring unit 106 instead of on the adhesive patch 108. The adhesive patch 108 may accordingly include an aperture or another opening whereby the ECG pads 202 of the cardiotoxicity monitoring unit 106 may contact the skin of the patient 100. As another illustration, the adhesive patch 108 may include one or more motion sensors, such as the 3D accelerometer 302.

Additionally, internally the wearable cardiotoxicity monitoring device 102 includes a controller operationally coupled to the plurality of externally applied biosignal sensors. For example, in the implementation illustrated in FIG. 5, the cardiotoxicity monitoring unit 106 of the wearable cardiotoxicity monitoring device 102 may include a microcontroller 310. The microcontroller 310 stores instructions specifying how measurements (e.g., ECG, accelerometer, RF, etc.) are taken, how obtained data are transmitted, how to relay a status of the cardiotoxicity monitoring unit 106, how/when the cardiotoxicity monitoring unit 106 can enter a sleep level, and/or the like. In implementations, the instructions may specify the conditions for performing certain types of measurements. For example, the instructions may specify that a sensor of the cardiotoxicity monitoring unit 106 and/or connected to the cardiotoxicity monitoring unit 106 may not commence measurements, such as RF measurements, ECG measurements, respiration rate measurements, and/or the like, unless the patient 100 is at rest or maintaining a certain posture (e.g., as indicated by data from the accelerometer 302). As another example, the instructions may identify the conditions that have to be fulfilled before any measurements can commence, such as a sufficient attachment level between the cardiotoxicity monitoring unit 106 and the adhesive patch 108, or a sufficient attachment level between the adhesive patch 108 and the surface of the patient's body to which the adhesive patch 108 is attached. In implementations, the microcontroller 310 may have internal and/or external non-volatile memory banks (e.g., memory 312) that can be used for measurement directories and data, scheduler information, and/or a log of actions and errors. This non-volatile memory may allow for retaining data and status information in the case of a total power down. In implementations, instead of a microcontroller 310 as described above, the cardiotoxicity monitoring unit 106 may include a microprocessor connected to a separate non-volatile memory, such as the memory 312.

The cardiotoxicity monitoring unit 106 may further be able to establish wireless communications channels with other devices, such as the portable gateway 110 and/or the remote server 104, using a telemetry or wireless communications circuit 314. For example, the wireless communications circuit 314 may be a Bluetooth® unit. Additionally, or alternatively, the wireless communications circuit 314 may include other modules facilitating other types of wireless communication (e.g., Wi-Fi, cellular, etc.). The cardiotoxicity monitoring unit 106 may transmit biosignal-based data, generated from one or more sensed biosignals, to the remote server 104 using the wireless communications circuit 314. In implementations, the cardiotoxicity monitoring unit 106 may transmit the biosignal-based data indirectly to the remote server 104, such as by transmitting the biosignal-based data to the portable gateway 110, with the portable gateway 110 transmitting the biosignal-based data to the remote server 104. In implementations, the cardiotoxicity monitoring unit 106 may instead transmit the biosignal-based data directly to the remote server 104.

In implementations, the patient 100 may instruct the wearable cardiotoxicity monitoring device 102 to record symptom events. For example, in embodiments, the patient 100 may provide a symptom input via the portable gateway 110 of the wearable cardiotoxicity monitoring device 102. As an illustration, the portable gateway 110 may include a visual display and a processor for the portable gateway 110 in communication with the visual display. As such, the portable gateway 110 may be configured to display, on the visual display, a screen that includes a list of potential cardiac-related symptoms to the patient 100. Example symptoms may include lightheadedness, a racing heart, fatigue, fainting, a fall, chest discomfort, a skipped heartbeat, and shortness of breath. The portable gateway 110 further may be configured to receive a selection by the patient 100 of one or more of the displayed potential cardiac-related symptoms. For instance, the visual display may include a touchscreen, where the patient 100 interacts with the touchscreen to select the cardiac-related symptom(s) the patient 100 is experiencing. As another example, the patient 100 may use an input device of the portable gateway 110, such as arrow buttons, a tracking pad, or a keyboard, to select the cardiac-related symptom(s) that the patient 100 is experiencing. The portable gateway 110 thus records the selected cardiac-related symptom(s) and the time the patient 100 submitted the cardiac-related symptom(s).

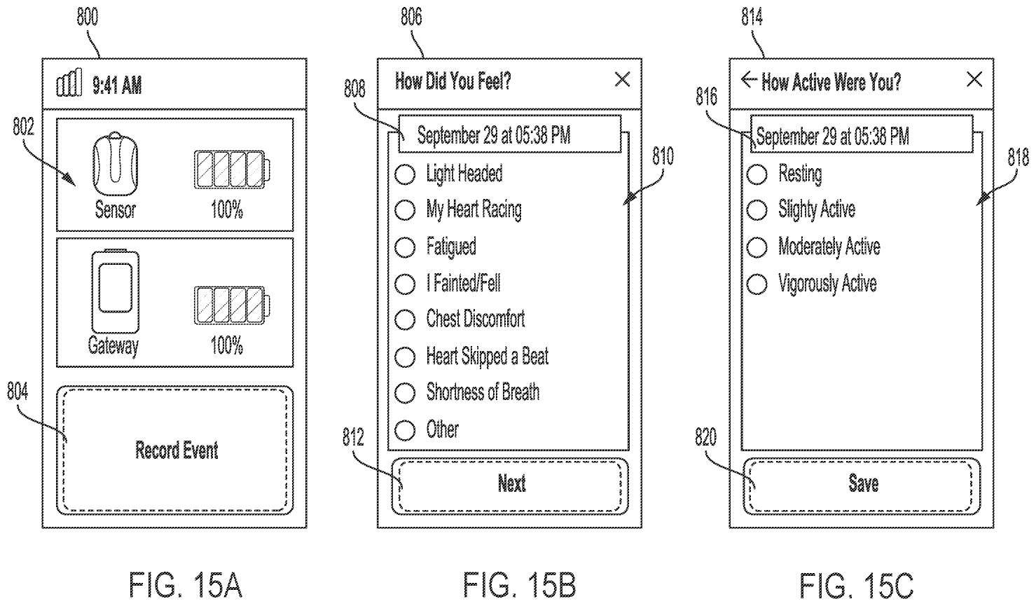

To illustrate, FIGS. 15A-15C show example screens that may be displayed on a visual display of the portable gateway 110 and used by the patient 100 to provide the symptom input. FIG. 15A shows an example home screen 800 that may be displayed by the portable gateway 110, for instance, as a default screen for the portable gateway 110. The home screen 800 may include information on the wearable cardiotoxicity monitoring device 102 such as battery level indicators 802 (e.g., for the portable gateway 110 and the cardiotoxicity monitoring unit 106). Additionally, the home screen 800 includes a “Record Event” button 804 that the patient 100 can press if the patient 100 is experiencing a suspected cardiac-related symptom.

In response to the patient 100 selecting the “Record Event” button 804, the portable gateway 110 may display a symptom screen 806, an example of which is shown in FIG. 15B. The symptom screen may include a subsection 808 listing the time and date that the patient 100 is reporting the suspected cardiac-related symptom (e.g., the time and date the patient 100 selected the “Record Event” button 804). In addition, the symptom screen 806 includes a list 810 of potential cardiac-related symptoms. As an illustration, in the example of FIG. 15B, the list 810 of potential cardiac-related symptoms may include Light-Headed,” “My Heart Racing,” “Fatigued,” “I Fainted/Fell,” “Chest Discomfort,” “Heart Skipped a Beat,” “Shortness of Breath,” and “Other” (e.g., in response to the selection of which the patient 100 is shown a screen or an additional text box into which the patient 100 may type their “other” symptom(s)). The symptom screen 806 may also include a “Next” button 812 that the patient 100 can press once the patient 100 has selected the suspected cardiac-related symptom(s) from the list 810.

In response to the patient 100 selecting the “Next” button 812, the portable gateway 110 may display an activity screen 814, an example of which is shown in FIG. 15C. The activity screen 814 may include a subsection 816 that again lists the time and date that the patient 100 is reporting the suspected cardiac-related symptom. The activity screen 814 also includes a list 818 of types of potential activities that the patient 100 may have been engaging in when the patient 100 started experiencing the recorded symptom(s). As shown in the example of FIG. 15C, the list 818 of potential types of activity may include “Resting,” “Slightly Active,” “Moderately Active,” and “Vigorously Active.” The activity screen 814 may also include a “Save” button 820 that the patient 100 can press once the patient 100 has selected the type of activity the patient 100 was experiencing from the list 818. Once the patient 100 has selected the “Save” button 820, the portable gateway 110 may transmit the selected cardiac-related symptom(s) from the list 810 of potential cardiac-related symptoms, the selected type of activity from the list 818 of potential types of activity, and the time and date of the symptom input.

In implementations, the example screens described above may be displayed on a user interface disposed on another component of the wearable cardiotoxicity monitoring device 102 aside from the portable gateway 110, e.g., on a housing of the cardiotoxicity monitoring unit 106. The patient 100 may thus provide the symptom input via the cardiotoxicity monitoring unit 106. For example, the front of the cardiotoxicity monitoring unit 106 (e.g., the face of the cardiotoxicity monitoring unit 106 facing away from the patient 100 when the cardiotoxicity monitoring unit 106 is being worn by the patient 100) may be configured to receive a patient input. As an illustration, the front of the cardiotoxicity monitoring unit 106 may include a button that the patient 100 can tap, or the cardiotoxicity monitoring unit 106 may sense changes in electrical charge on the front surface by the patient 100 tapping on the front surface. The patient 100 can then provide a symptom input to the cardiotoxicity monitoring unit 106 via the front face of the cardiotoxicity monitoring unit 106. An example process for providing a symptom input to the cardiotoxicity monitoring unit 106 may include the patient 100 (1) remaining still, (2) double tapping the front face of the cardiotoxicity monitoring unit 106 with the palm of the patient's hand, and (3) waiting for a beeping sound from the cardiotoxicity monitoring unit 106, which indicates that the symptom input has been recorded. If the beeping sound does not occur, the patient 100 may need to re-tap the cardiotoxicity monitoring unit 106 to record the symptom input.

In implementations, the wearable cardiotoxicity monitoring device 102 may be configured to record symptoms from someone other than the patient 100. For example, the wearable cardiotoxicity monitoring device 102 may record symptoms from a caregiver of the patient 100. In some cases, the caregiver may be a physician, nurse, or other healthcare provider overseeing the patient 100. In some cases, the caregiver may be a family member of the patient 100 or another at-home caregiver of the patient 100. As an illustration, an at-home caregiver may input a symptom of the patient 100 using the portable gateway 110 as described above.

Furthermore, in addition to recording the symptom input from the patient 100, the wearable cardiotoxicity monitoring device 102 is configured to record one or more biosignal segments associated with the symptom input. In implementations, the wearable cardiotoxicity monitoring device 102 is configured to record the one or more biosignal segments within a predetermined time period before and after the symptom input. For example, the wearable cardiotoxicity monitoring device 102 may record the one or more biosignal segments 30 seconds, 1 minute, 2 minutes, 3 minutes, 4 minutes, 5 minutes, etc. before and/or after the symptom input. In some cases, the wearable cardiotoxicity monitoring device 102 may record the one or more biosignal segments for a predetermined time period after the symptom input. In some cases, the wearable cardiotoxicity monitoring device 102 may record the one or more biosignal segments for a first predetermined time period before the symptom input and a second predetermined time period after the symptom input. The first predetermined time period may be the same as the second predetermined time period, or the first predetermined time period may be different from the second predetermined time period. As an illustration, the wearable cardiotoxicity monitoring device 102 may record the one or more biosignal segments for 30 seconds before the symptom input and 1 minute after the symptom input. The biosignal(s) for the one or more biosignal segments may include any of the biosignal(s) discussed above, such as ECG signals, cardiovibrational signals, respiration signals, thoracic fluid signals, and/or the like. In implementations, the wearable cardiotoxicity monitoring device 102 may also record one or more additional signal segments. For example, the wearable cardiotoxicity monitoring device 102 may record accelerometer signal segments containing activity level and posture information for the patient 100. The wearable cardiotoxicity monitoring device 102 may be configured to transmit data associated with the symptom event (e.g., including an indication of the symptom event and/or the one or more biosignal segments recorded in association with the symptom event) to the remote server 104 as part of the biosignal-based data, described in further detail below.

In implementations, at least some of the functionality described above as occurring on the cardiotoxicity monitoring unit 106 may occur on the portable gateway 110. As an example, at least some of the components or processes described above as being located at and/or performed by the cardiotoxicity monitoring unit 106 may be located at and/or performed by the portable gateway 110. Thus, the controller of the wearable cardiotoxicity monitoring device 102 may be implemented through a combination of the cardiotoxicity monitoring unit 106 and the portable gateway 110.

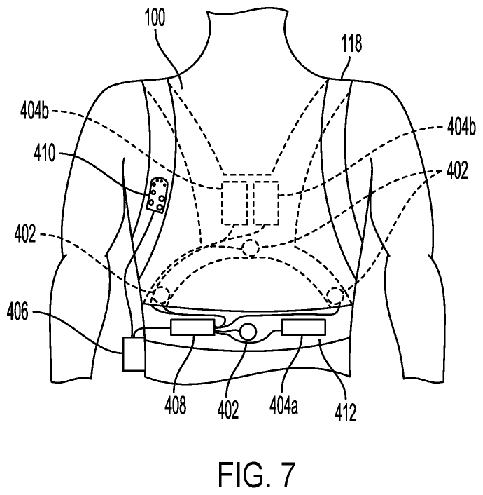

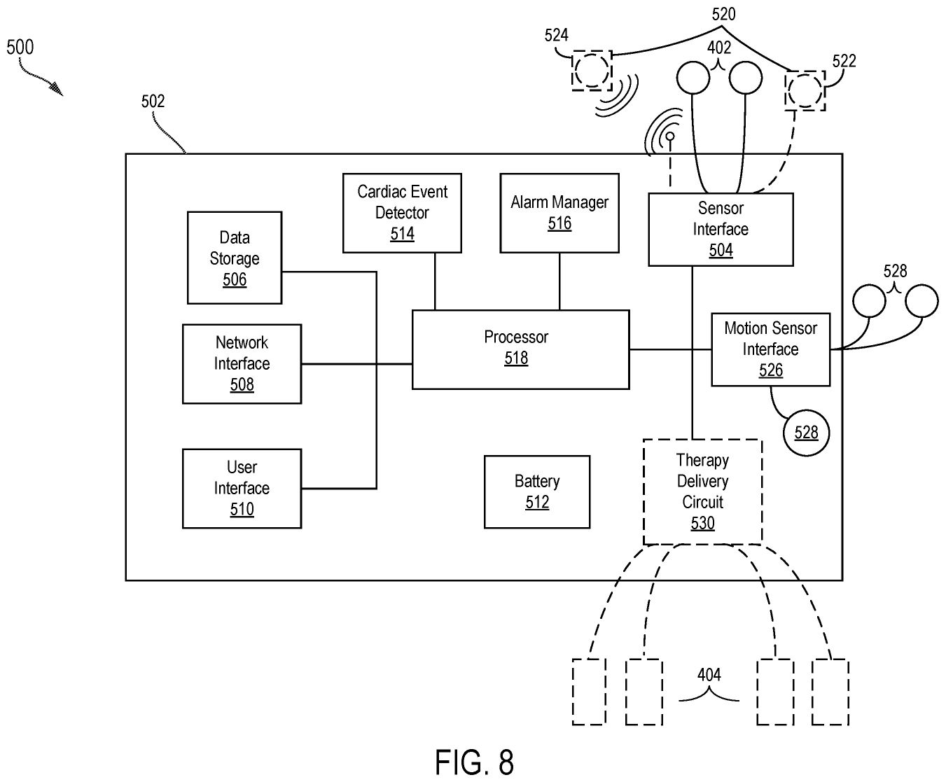

In implementations, the wearable cardiotoxicity monitoring device 102 may be configured as a different wearable device from the example embodiments illustrated in FIGS. 1-5. For instance, FIG. 6 illustrates another example of a wearable cardiotoxicity monitoring system for monitoring cardiotoxicity biosignal markers in a patient 100, according to implementations disclosed herein. The wearable cardiotoxicity monitoring system of FIG. 6 includes another embodiment of the wearable cardiotoxicity monitoring device 102 configured for long-term continuous wear by the patient 100, where the wearable cardiotoxicity monitoring device 102 is in communication with the remote server 104. The wearable cardiotoxicity monitoring device 102 according to FIG. 6 is a garment-based monitoring device 118 configured to be worn about the patient's torso, where various electronic components are temporarily and/or permanently mounted to the garment of the garment-based monitoring device 118 (as described in further detail with respect to FIG. 7 below). Such a garment-based monitoring device 118 can be, for example, capable of and designed for moving with the patient 100 as the patient 100 goes about their daily routine. For example, the garment-based monitoring device 118 as described herein with respect to FIG. 6 (as well as FIGS. 7 and 8 below) can be a wearable cardioverter defibrillator, or wearable defibrillator, configured to be bodily-attached to the patient 100. In one example scenario, such wearable defibrillators can be worn nearly continuously or substantially continuously for a weck, two weeks, a month, or two or three months at a time. During the period of time in which it is worn by the patient 100, the wearable defibrillator can be configured to continuously or substantially continuously monitor the vital signs of the patient 100 and, upon determining that the patient 100 is experiencing a treatable arrhythmia, deliver one or more therapeutic electrical pulses to the patient 100. For example, such therapeutic shocks can be pacing, defibrillation, cardioversion, or transcutaneous electrical nerve stimulation (TENS) pulses.

The wearable cardiotoxicity monitoring system shown in FIG. 6 (including the remote server 104, technician interface(s) 114, and caregiver interface(s) 116) may generally function similarly to the wearable cardiotoxicity monitoring system shown and described above with respect to FIG. 1. Similar to the example wearable cardiotoxicity monitoring device 102 shown in FIG. 2, the example wearable cardiotoxicity monitoring device 102 shown in FIG. 6 is configured for long-term and/or extended use or wear by, or attachment or connection to, the patient 100, as discussed above. Such substantially or nearly continuous use, monitoring, or wear similar to the usages described above may nonetheless be considered continuous use, monitoring, or wear.

FIG. 7 illustrates the garment-based monitoring device 118 in more detail, according to various implementations. As shown in FIG. 7, the garment-based monitoring device 118 can include one or more of the following: a garment 400 configured to be worn around the patient's torso, one or more externally applied sensors configured to output one or more physiological signals, one or more therapy electrodes 404a and 404b (collectively referred to herein as therapy electrodes 404), a medical device controller 406, a connection pod 408, a patient interface pod 410, a belt 412, or any combination of these components. In implementations, the externally applied sensors may include ECG electrodes 402 configured to sense one or more electrical signals indicative of ECG activity from the skin surface of the patient 100. In implementations, the externally applied sensors may include at least one non-ECG physiological sensor configured to sense a non-ECG biosignal of the patient 100. Such non-ECG physiological sensors may be similar to the non-ECG sensors described above with reference to the cardiotoxicity monitoring unit 106 and the adhesive patch 108. For example, the garment-based monitoring device 118 may include or be connected to a cardiovibration sensor configured to sense cardiovibrations of the patient 100, a respiration sensor configured to sense respirations of the patient 100, a thoracic fluid sensor configured to sense thoracic fluid levels in the patient 100, a blood pressure sensor configured to sense a blood pressure of the patient 100, and/or the like. Other types of non-ECG physiological sensors may include a wear state sensor configured to detect a wear state of the garment-based monitoring device 118, a bioacoustics sensor (e.g., a microphone) configured to generate bioacoustics signals for the heart of the patient 100, and/or the like.

In examples, at least some of the components of the garment-based monitoring device 118 (e.g., at least a portion of the biosignal sensors, including the ECG electrodes 402 and/or non-ECG physiological sensors; at least a portion of the therapy electrodes 404; etc.) are configured to be mounted on or affixed to the garment 400, such as by mating hooks, hook-and-loop fabric strips, receptacles (e.g., pockets), and the like. For example, the ECG electrodes 402 may be mounted on the garment 400 by hook-and-loop fabric strips on the ECG electrodes 402 and the garment 400, and the therapy electrodes 404 may be mounted on the garment 400 by being inserted into receptacles of the garment 400 (e.g., into pockets of the garment 400). In examples, at least some of the components of the garment-based monitoring device 118 (e.g., at least a portion of the biosignal sensors, including the ECG electrodes 402 and/or non-ECG physiological sensors; at least a portion of the therapy electrodes 404; etc.) can be permanently integrated into the garment 400, such as being sewn into or permanently adhered onto the garment 400. In examples, at least some of the components may be connected to each other through external cables, through sewn-in connections (e.g., wires woven into the fabric of the garment 400 or between layers of the garment 400), through conductive fabric of the garment 400, and/or the like.

The medical device controller 406 can be operatively coupled to the ECG electrodes 402, which as described can be affixed to the garment 400 (e.g., assembled into the garment 400 or removably attached to the garment 400, for example, using hook-and-loop fasteners) or permanently integrated into the garment 400. In implementations, the medical device controller 406 is also operatively coupled to the therapy electrodes 404. The therapy electrodes 404 may be similarly assembled into the garment 400 (e.g., into pockets or other receptacles of the garment 400) or permanently integrated into the garment 400. As shown in FIG. 4, the ECG electrodes 402 and/or the therapy electrodes 404 can be operatively coupled to the medical device controller 406 through the connection pod 408. Component configurations other than those shown in FIG. 7 are also possible. For example, the ECG electrodes 402 can be configured to be attached at various positions about the body of the patient 100. In implementations, at least one of the ECG electrodes 402 and/or at least one of the therapy electrodes 404 can be included on a single integrated patch and adhesively applied to the patient's body. In implementations, at least one of the ECG electrodes 402 and/or at least one of the therapy electrodes 404 can be included in multiple patches and adhesively applied to the patient's body. Such patches may be in wired (e.g., directly or via the connection pod 408) or wireless connection with the medical device controller 406. Similar implementations may also be extended to the non-ECG physiological sensors of the garment-based monitoring device 118, such as a motion sensor, a thoracic fluid sensor, etc. Alternatively, in implementations, at least some of the non-ECG physiological sensor may be implemented in the medical device controller 406. For instance, the housing of the medical device controller 406 may also house a motion sensor, a thoracic fluid sensor, etc., as described in further detail with respect to FIG. 8.

The ECG electrodes 402 are configured to detect one or more cardiac signals, such as electrical signals indicative of ECG activity from a skin surface of the patient 100. In implementations, the ECG electrodes 402 of the garment-based monitoring device 118 may be configured similarly to the ECG electrodes 202 of the adhesive patch 108 described above. In implementations, the therapy electrodes 404 can also be configured to include sensors that detect ECG signals as well as, or in the alternative from, other physiological signals from the patient 100. The connection pod 408 can, in various examples, include a signal processor configured to amplify, filter, and digitize these cardiac signals prior to transmitting the cardiac signals to the medical device controller 406.

The therapy electrodes 404 can be configured to deliver one or more therapeutic cardioversion/defibrillation shocks to the body of the patient 100 when the medical device controller 406 determines that such treatment is warranted based on the signals detected by the ECG electrodes 402 and processed by the medical device controller 406. Example therapy electrodes 404 can include conductive metal electrodes such as stainless-steel electrodes. In implementations, the therapy electrodes 404 may also include one or more conductive gel deployment devices configured to deliver conductive gel between the metal electrode and the patient's skin prior to delivery of a therapeutic shock.

In implementations, the medical device controller 406 may also be configured to warn the patient 100 prior to the delivery of a therapeutic shock, such as via output devices integrated into or connected to the medical device controller 406, the connection pod 408, and/or the patient interface pod 410. The warning may be auditory (e.g., a siren alarm and/or a voice instruction indicating that the patient 100 is going to be shocked issued via a speaker of the medical device controller and/or patient interface pod 410), visual (e.g., flashing lights on the medical device controller 406), haptic (e.g., a tactile, buzzing alarm generated by the connection pod 408), and/or the like. If the patient 100 is still conscious, the patient 100 may be able to delay or stop the delivery of the therapeutic shock. For example, the patient 100 may press one or more buttons on the patient interface pod 410 and/or the medical device controller 406 to indicate that the patient 100 is still conscious. In response to the patient 100 pushing the one or more buttons, the medical device controller 406 may delay or stop the delivery of the therapeutic shock.

In implementations, a garment-based monitoring device 118 as described herein can be configured to switch between a therapeutic mode and a monitoring mode such that, when in the monitoring mode, the garment-based monitoring device 118 is configured to only monitor the patient 100 (e.g., not provide or perform any therapeutic functions). For example, in such implementations, therapeutic components such as the therapy electrodes 404 and associated circuitry may be decoupled from (or coupled to) or switch out of (or switched into) the garment-based monitoring device 118. As an illustration, a garment-based monitoring device 118 can have optional therapeutic elements (e.g., defibrillation and/or pacing electrode components and associated circuitry) that are configured to operate in a therapeutic mode. The optional therapeutic elements may be physically decoupled from the garment-based monitoring device 118 as a means to convert the garment-based monitoring device 118 from a therapeutic mode into a monitoring mode. Alternatively, the optional therapeutic elements may be deactivated (e.g., by means of a physical or software switch), essentially rendering the garment-based monitoring device 118 as a monitoring-only device for a specific physiological purpose for the particular patient 100. As an example of a software switch, an authorized person may be able to access a protected user interface of the garment-based monitoring device 118 and select a preconfigured option or perform some other user action via the user interface to deactivate the therapeutic elements of the garment-based monitoring device 118.

FIG. 8 illustrates a sample component-level view of a medical device controller 500 included in a garment-based monitoring device 118. The medical device controller 500 is an example of the medical device controller 406 shown in FIG. 7 and described above. As shown in FIG. 8, the medical device controller 500 may include a housing 502 configured to house a number of electronic components, including a sensor interface 504, a data storage 506, a network interface 508, a user interface 510, at least one battery 512 (e.g., positioned within a battery chamber configured for such a purpose), a cardiac event detector 514, an alarm manager 516, at least one processor 518, and a therapy delivery circuit 530. As described above, in some implementations, the garment-based monitoring device 118 may include like components to those described above but may not include therapeutic components. That is, in some implementations, the garment-based monitoring device 118 can include ECG monitoring components and not provide therapy to the patient 100. Accordingly, the garment-based monitoring device 118 may not include the therapy delivery circuit 530 and the therapy electrodes 404 (shown in dotted lines), or the therapy delivery circuit 530 and the therapy electrodes 404 may be disconnectable from the rest of the garment-based monitoring device 118.

In implementations, the processor 518 includes one or more processors (or one or more processor cores) that are each configured to perform a series of instructions that result in the manipulation of data and/or the control of the operation of the other components of the medical device controller 500. In implementations, when executing a specific process (e.g., monitoring sensed biosignals), the processor 518 can be configured to make specific logic-based determinations based on input data received. The processor 518 may be further configured to provide one or more outputs that can be used to control or otherwise inform subsequent processing to be carried out by the processor 518 and/or other processors or circuitry to which the processor 518 is communicably coupled. Thus, the processor 518 reacts to a specific input stimulus in a specific way and generates a corresponding output based on that input stimulus. In example cases, the processor 518 can proceed through a sequence of logical transitions in which various internal register states and/or other bit cell states internal or external to the processor 518 may be set to logic high or logic low.

As referred to herein, the processor 518 can be configured to execute a function where software is stored in a data store (e.g., the data storage 506) coupled to the processor 518, the software being configured to cause the processor 518 to proceed through a sequence of various logic decisions that result in the function being executed. The various components that are described herein as being executable by the processor 518 can be implemented in various forms of specialized hardware, software, or a combination thereof. For example, the processor 518 can be a digital signal processor (DSP) such as a 24-bit DSP processor. As another example, the processor 518 can be a multi-core processor, e.g., having two or more processing cores. As another example, the processor 518 can be an Advanced RISC Machine (ARM) processor, such as a 32-bit ARM processor. The processor 518 can execute an embedded operating system and further execute services provided by the operating system, where these services can be used for file system manipulation, display and audio generation, basic networking, firewalling, data encryption, communications, and/or the like.

The data storage 506 can include one or more of non-transitory media, such as flash memory, solid state memory, magnetic memory, optical memory, cache memory, combinations thereof, and others. The data storage 506 can be configured to store executable instructions and data used for operation of the medical device controller 500. In implementations, the data storage 506 can include sequences of executable instructions that, when executed, are configured to cause the processor 518 to perform one or more functions. Additionally, the data storage 506 can be configured to store information such as sensed biosignals and biosignal-based data, as well as data from other sensors of the garment-based monitoring device 118, as described in further detail below.

In examples, the network interface 508 can facilitate the communication of information between the medical device controller 500 and one or more devices or entities over a communications network. For example, the network interface 508 can be configured to communicate with the remote server 104 or other similar computing device. As an illustration, using the network interface 508, the garment-based monitoring device 118 may transmit biosignal-based data, based on the one or more sensed biosignals, to the remote server 104. In implementations, the network interface 508 can include communications circuitry for transmitting data in accordance with a Bluetooth® wireless standard for exchanging such data over short distances to an intermediary device(s) (e.g., a base station, “hotspot” device, smartphone, tablet, portable computing device, and/or other device in proximity with the garment-based monitoring device 118, such as a device similar to the portable gateway 110). The intermediary device(s) may in turn communicate the data to the remote server 104 over a broadband cellular network communications link. The communications link may implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In some implementations, the intermediary device(s) may communicate with the remote server 104 over a Wi-Fi communications link based on the IEEE 802.11 standard. In implementations, the network interface 508 may be configured to instead communicate directly with the remote server 104 without the use of intermediary device(s). In such implementations, the network interface 508 may use any of the communications links and/or protocols provided above to communicate directly with the remote server 104.

The sensor interface 504 can include physiological signal circuitry that is coupled to one or more externally applied sensors 520. The externally applied sensors 520 may include, for example, one or more externally applied biosignal sensors configured to sense one or more biosignals from the patient 100. In implementations, the sensor interface 504 may also be coupled to one or more additional sensors and/or receive additions signals from the externally applied biosignal sensors. As shown, the sensors may be coupled to the medical device controller 500 via a wired or wireless connection. The externally applied sensors 520 may include the ECG electrodes 202 configured to sense one or more electrical signals indicative of ECG activity from the skin surface of the patient 100, as well as one or more non-biosignal sensors such as a cardiovibration sensor 522 and a tissue fluid monitor 524 (e.g., configured similarly to the thoracic fluid sensor implemented through the at least one RF antenna 304a, 304b and RF circuitry 306 discussed above with reference to FIG. 5). Other examples of externally applied sensors 520 may include a respiration sensor, a bioacoustics sensor, a blood pressure sensor, a temperature sensor, a pressure sensor, a humidity sensor, a P-wave sensor (e.g., a sensor configured to monitor and isolate P-waves within an ECG waveform), an oxygen saturation sensor (e.g., implemented through photoplethysmography, such as through light sources and light sensors configured to transmit light into the patient's body and receive transmitted and/or reflected light containing information about the patient's oxygen saturation), and so on.

The one or more cardiovibration sensors 522 can be configured to detect cardiac or pulmonary vibration information. The one or more cardiovibration sensors 522 can transmit information descriptive of the cardiovibrations (and other types of sensed vibrations) to the sensor interface 504 for subsequent analysis. For example, the one or more cardiovibration sensors 522 can detect the patient's heart valve vibration information (e.g., from opening and closing during cardiac cycles). As a further example, the one or more cardiovibration sensors 522 can be configured to detect cardiovibrational signal values including one or more of S1, S2, S3, and S4 cardiovibrational biomarkers. From these cardiovibrational signal values or heart vibration values, certain heart vibration metrics may be calculated (e.g., at the garment-based monitoring device 118 and/or at the remote server 104). These heart vibration metrics may include one or more of electromechanical activation time (EMAT), average EMAT, percentage of EMAT (% EMAT), systolic dysfunction index (SDI), or left ventricular systolic time (LVST). The one or more cardiovibration sensors 522 can also be configured to detect heart wall motion, for instance, by placement of the sensor in the region of the apical beat. In implementations, the one or more cardiovibration sensors 522 can include a vibrational sensor configured to detect vibrations from the patient's cardiac and pulmonary system and provide an output signal responsive to the detected vibrations of a targeted organ. For example, the one or more cardiovibration sensors 522 may be configured to detect vibrations generated in the trachea or lungs due to the flow of air during breathing. In implementations, additional physiological information can be determined from pulmonary-vibrational signals such as, for example, lung vibration characteristics based on sounds produced within the lungs (e.g., stridor, crackle, etc.). In implementations, the one or more cardiovibration sensors 522 can include a multi-channel accelerometer, for example, a three-channel accelerometer configured to sense movement in each of three orthogonal axes such that patient movement/body position can be detected and correlated to detected cardiovibrations information.

In implementations, the sensor interface 504 may be connected to one or more motion sensors (e.g., one or more accelerometers, gyroscopes, magnetometers, ballistocardiographs, etc.) as part of the externally applied sensors 520. In implementations, the medical device controller 500 may include a motion detector interface, either implemented separately or as part of the sensor interface 504. For instance, as shown in FIG. 8, the medical device controller 500 may include a motion sensor interface 526 operatively coupled to one or more motion detectors 528 configured to generate motion data, for example, indicative of physical activity performed by the patient 100 and/or physiological information internal to the patient 100. Examples of a motion detector may include a 1-axis channel accelerometer, 2-axis channel accelerometer, 3-axis channel accelerometer, multi-axis channel accelerometer, gyroscope, magnetometer, ballistocardiograph, and the like. As an illustration, the motion data may include accelerometer counts indicative of physical activity performed by the patient 100, accelerometer counts indicative of respiration rate of the patient 100, accelerometer counts indicative of posture information for the patient 100, accelerometer counts indicative of cardiovibrational information for the patient 100, and/or the like.

The motion sensor interface 526 is configured to receive one or more outputs from the motion sensors 528. The motion sensor interface 526 can be further configured to condition the output signals by, for example, converting analog signals to digital signals (if using an analog motion sensor), filtering the output signals, combining the output signals into a combined directional signal (e.g., combining each x-axis signal into a composite x-axis signal, combining each y-axis signal into a composite y-axis signal, and combining each z-axis signal into a composite z-axis signal), and/or the like. In examples, the motion sensor interface 526 can be configured to filter the signals using a high-pass or band-pass filter to isolate the acceleration of the patient due to movement from the component of the acceleration due to gravity. Additionally, the motion sensor interface 526 can configure the outputs from the motion sensor 528 for further processing. For example, the motion sensor interface 526 can be configured to arrange the output of an individual motion sensor 528 as a vector expressing acceleration components of the x-axis, the y-axis, and the z-axis of the motion sensor 528. The motion sensor interface 526 can thus be operably coupled to the processor 518 and configured to transfer the output and/or processed motion signals from the motion sensors 528 to the processor 518 for further processing and analysis.

In implementations, the one or more motion sensors 528 can be integrated into one or more components of the garment-based monitoring device 118, either within the medical device controller 500 or external to the medical device controller 500 as shown in FIG. 8. For instance, in some implementations, the one or more motion detectors 528 may be located in or near the ECG electrodes 202. In some implementations, the one or more motion detectors 528 may be located elsewhere on the garment-based monitoring device 118. For example, a motion detector 528 may be integrated into the medical device controller 500 (e.g., such that the one or more motion detectors 528 would be located within the housing 502 of the medical device controller 500, as shown in FIG. 8). In some implementations, a motion detector 528 may be integrated into another component of the garment-based monitoring device 118, such as a therapy electrode 404, the connection pod 408, and/or the like. In some implementations, a motion detector 528 can be integrated into an adhesive ECG sensing and/or therapy electrode patch.

As described above, the sensor interface 504 and the motion sensor interface 526 can be coupled to any one or combination of biosignal sensors and/or other sensors to receive patient data indicative of patient parameters. Once data from the sensors has been received by the sensor interface 504 and/or the motion sensor interface 526, the data can be directed by the processor 518 to an appropriate component within the medical device controller 500. For example, ECG signals collected by the ECG sensors 402 may arrive at the sensor interface 504, and the sensor interface 504 may transmit the ECG signals to the processor 518, which, in turn, relays the patient's ECG data to the cardiac event detector 514 (e.g., described in further detail below). The sensor data can also be stored in the data storage 506 and/or transmitted to the remote server 104 via the network interface 508. For instance, the processor 518 may transmit biosignal-based data (based on the one or more sensed biosignals) to the remote server 104. As another example, the processor 518 may transmit additional sensor data, such as data associated with the patient's motion or body position, to the remote server 104.

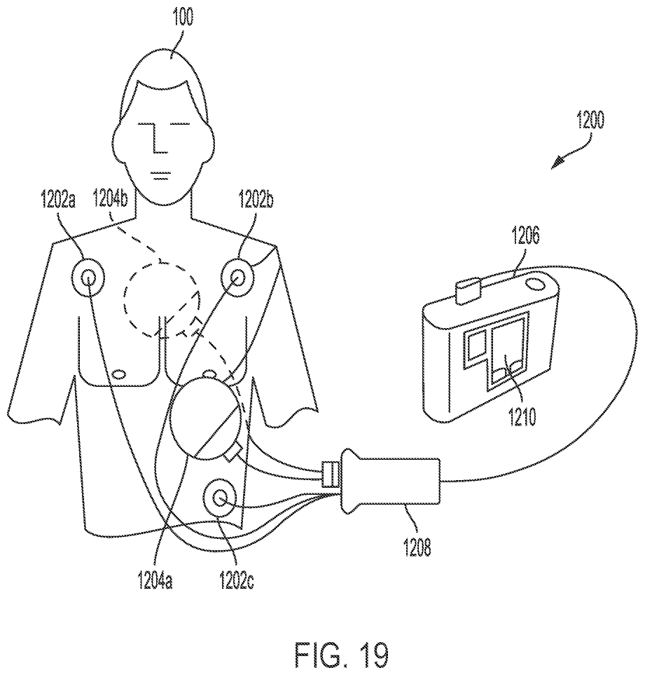

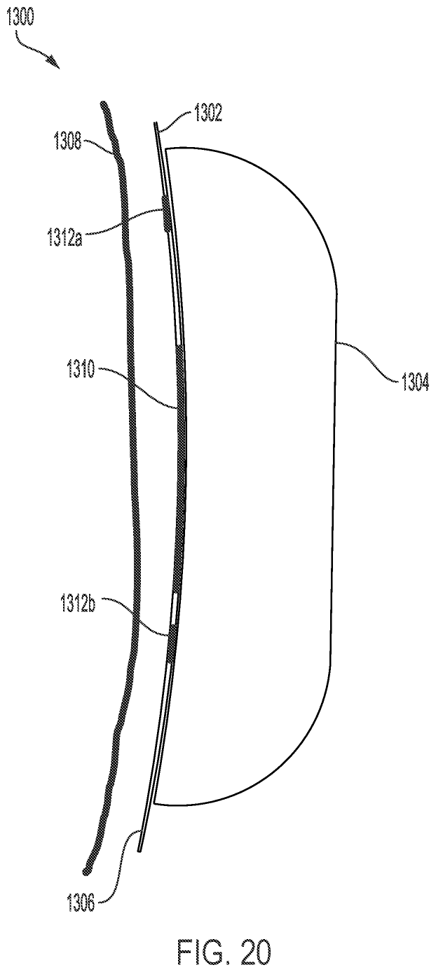

The embodiments of the wearable cardiotoxicity monitoring device 102 described above, including the cardiotoxicity monitoring unit 106, adhesive patch 108, and portable gateway 110, and the garment-based monitoring device 118, are examples of the wearable cardiotoxicity monitoring device 102. However, other embodiments of a wearable cardiotoxicity monitoring device 102 may also be used with the systems and methods described herein. Examples of other embodiments of the wearable cardiotoxicity monitoring device 102, for instance, are described below with respect to FIGS. 19 and 20.

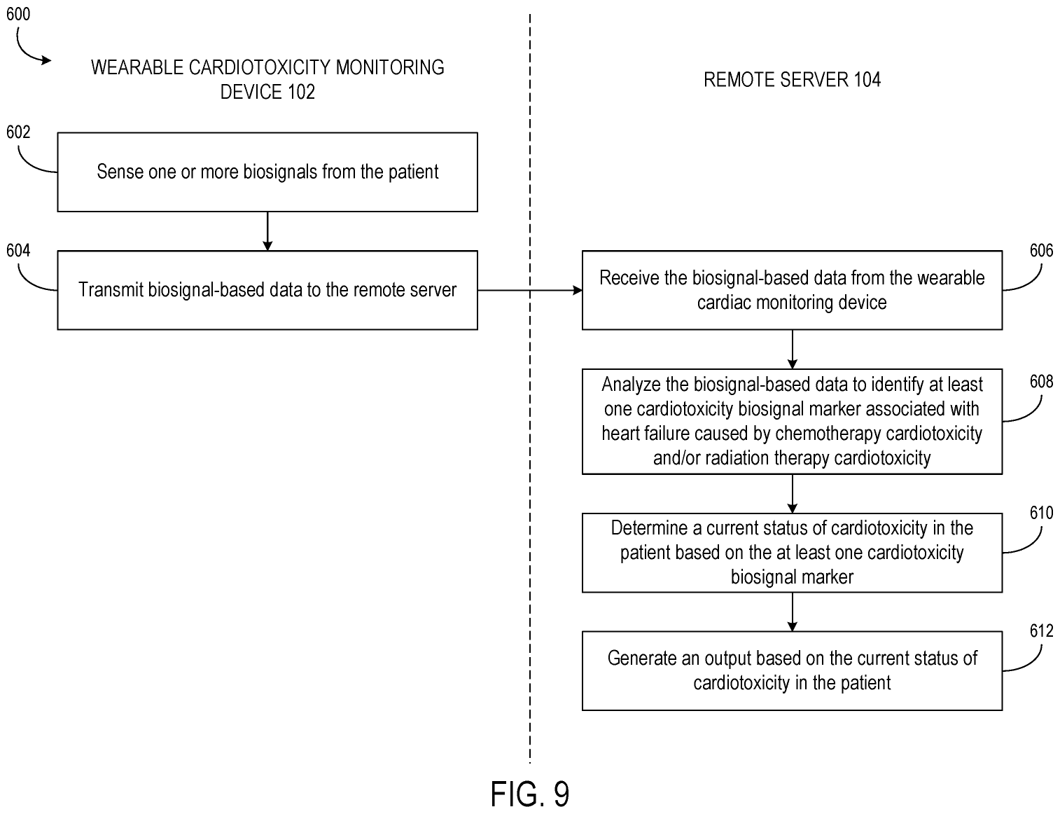

FIG. 9 illustrates a sample process flow for monitoring cardiotoxicity biosignal markers in oncology patients. The sample process 600 shown in FIG. 9 can be implemented by the wearable cardiotoxicity monitoring device 102 being used by the patient 100 and the remote server 104, as shown in FIG. 9. The wearable cardiotoxicity monitoring device 102 is configured to sense one or more biosignals from the patient 100 at step 602. In implementations, the wearable cardiotoxicity monitoring device 102 may be configured to sense one or more electrical signals indicative of ECG activity from the skin surface of the patient 100. For example, in embodiments of the wearable cardiotoxicity monitoring device 102 including the cardiotoxicity monitoring unit 106 and the adhesive patch 108, the wearable cardiotoxicity monitoring device 102 may sense the electrical signals indicative of ECG activity using the ECG pads 202 of the adhesive patch 108 in electrical communication with the ECG circuits 300 of the cardiotoxicity monitoring unit 106. As another example, in embodiments of the wearable cardiotoxicity monitoring device 102 including the garment-based monitoring device 118, the wearable cardiotoxicity monitoring device 102 may sense the electrical signals indicative of ECG activity using the ECG electrodes 402 and the sensor interface 504 of the medical device controller 500.

In implementations, the wearable cardiotoxicity monitoring device 102 may also be configured to sense non-ECG biosignals of the patient 100, such as signals indicative of cardiovibrations, bioacoustics, respiration, thoracic fluid level, blood pressure, and/or the like. In implementations, the wearable cardiotoxicity monitoring device 102 may be configured to sense one or more additional types of signals associated with the patient 100 (e.g., non-biosignals associated with the patient 100). For instance, the wearable cardiotoxicity monitoring device 102 may sense motion signals associated with patient movement. These motion signals may, for example, simultaneously include information about the patient's body posture, activity level, respiration rate, etc., or these motion signals may include different signals for device tilt (e.g., corresponding to the patient's body posture) and patient movement (e.g., corresponding to the patient's activity level, respiration, etc.).

The wearable cardiotoxicity monitoring device 102 is configured to transmit biosignal-based data, based on the one or more sensed biosignals, to the remote server 104 at step 604. In implementations, the biosignal-based data may include, or be processable to produce, an ECG of the patient 100, cardiovibrational data for the patient 100, bioacoustics data for the patient 100, a heart rate (or series of heart rates) of the patient 100, respiration data for the patient 100, RF-based measurements associated with a thoracic fluid level of the patient 100, the thoracic fluid level of the patient 100, and/or the like. In implementations, the wearable cardiotoxicity monitoring device 102 is further configured to transmit additional types of sensor data to the remote server 104. For example, the wearable cardiotoxicity monitoring device 102 may transmit accelerometer data based on one or more motion signals sensed by an accelerometer (e.g., the accelerometer 302 of the cardiotoxicity monitoring unit 106, the one or more motion sensors 528 of the garment-based monitoring device 118, etc.).

In implementations, the wearable cardiotoxicity monitoring device 102 is configured to transmit raw or minimally processed sensed biosignals to the remote server 104 as the biosignal-based data. In implementations, the wearable cardiotoxicity monitoring device 102 may perform at least some processing and/or analysis of the sensed biosignals, and the wearable cardiotoxicity monitoring device 102 may transmit the results of the processed and/or analyzed biosignals to the remote server 104 as the biosignal-based data. For example, the wearable cardiotoxicity monitoring device 102 (e.g., at the cardiotoxicity monitoring unit 106, at the portable gateway 110, at the connection pod 408, and/or at the medical device controller 406, depending on the implementation) may filter at least some of the sensed biosignals to remove noise (e.g., audible noise, background noise, etc.) and/or motion-based artifacts. As an example, processor 518 may be configured to use an adaptive filtering process where an accelerometer signal (e.g., from the one or more motion sensors 528) is used as a reference corresponding to the motion-based artifact. In such a scheme, an adaptive filter is designed to optimize non-linear, time-varying filters by updating its weight vector based on minimizing an error that is fed back into the system.

Adaptive interference canceling can be modeled as a feedback control system where d(n) is a desired output, y(n) is an output of the filter, and e(n) is an error or difference between the signals d(n) and y(n) at a summing junction. The error signal gets fed to an adaptive algorithm after each iteration. The reference accelerometer input contains the motion or audio artifact noise that is also contained in the primary biosignal input. The adaptive filtering system is configured to get y(n), the output of the filter, as close to the desired signal d(n) as possible.

As another example, the wearable cardiotoxicity monitoring device 102 may discard undesirable biosignal measurements. To illustrate, the wearable cardiotoxicity monitoring device 102 may discard measurements taken when the patient 100 was engaging in an activity level above a predetermined threshold (e.g., as determined from accelerometer counts sensed by the accelerometer 302 or the one or more motion sensors 528) or measurements taken when the patient's body posture was above or below a predetermined threshold (e.g., as determined from recordings of gravity exerted on the accelerometer 302 or on the one or more motion sensors 528).

An illustration of a method for discarding undesirable biosignal measurements is provided as follows. As an example, a received biosignal can be segmented into a plurality of segments of a predefined duration, e.g., 5 seconds, 10 seconds, or other user- or technician-defined duration. For each of the plurality of segments, a statistical measure is established. For example, the statistical measure can be a median, mode, mean, standard deviation, or other such measure representative of a central tendency of the corresponding section. The calculated statistical measure is then compared to a predetermined threshold central value or a predetermined threshold central value range.

In examples, the predetermined threshold central value or predetermined threshold central value range is estimated during a baselining phase. In examples, the predetermined threshold central value predetermined threshold central value range is automatically determined for each patient 100 during the baselining period after the patient 100 initially wears the wearable cardiotoxicity monitoring device 102, e.g., for the first 1 minute, 5 minutes, 10 minutes, 30 minutes, 1 hour, or other preset baselining period of time. During this period, the predetermined threshold central value or predetermined threshold central value range is calculated from a plurality of segments of the biosignals as the statistical measure in a manner described above. Statistical measures for multiple segments are combined into a signal measure as the predetermined threshold central value. Additionally or alternatively, the statistical measures are used to establish a predetermined threshold central value range by selecting the smallest statistical measure as the lower end of the range and the largest statistical measure as the upper end of the range.

In examples, the predetermined threshold central value or predetermined threshold central value range is calculated based on a preset parameter provided via a user interface control by, e.g., a patient, a physician, a caregiver, or a technician. In implementations, the predetermined threshold central value or predetermined threshold central value range can be remotely set via a communication from the remote server 104. In scenarios, a physician may prescribe noise tolerance for a patient's biosignals, and the predetermined threshold central value or predetermined threshold central value range can be based on the prescribed noise tolerance. As an illustration, a physician may be presented with a noise tolerance scale ranging from 1-10, wherein 1 represents low noise tolerance (e.g., aggressive noise filtering) and 10 represents high noise tolerance (e.g., raw signal pass through or minimal noise filtering). In examples, the predetermined threshold central value or predetermined central value range is provided in units of dB, e.g., as a ratio of an absolute value of signal amplitude, frequency, or other predetermined signal parameter, relative to a reference value. In examples, when the calculated statistical measure transgresses the predetermined threshold central value or predetermined central value range, the corresponding biosignal segment is deemed corrupted and ignored or discarded.

As another example, the wearable cardiotoxicity monitoring device 102 may process at least some of the sensed biosignals to generate information about the patient 100 and the patient's health. To illustrate, the wearable cardiotoxicity monitoring device 102 may use RF measurements taken by the at least one RF antenna 304a, 304b and the RF circuitry 306 of the cardiotoxicity monitoring unit 106 (or the similarly implemented tissue fluid sensor 524 of the garment-based monitoring device 118) to determine a thoracic fluid level in the patient 100. The thoracic fluid level may be a relative thoracic fluid level (e.g., relative to a baseline taken when the patient 100 began oncology treatment) or an absolute thoracic fluid level. The wearable cardiotoxicity monitoring device 102 may then transmit the determined thoracic fluid level to the remote server 104. As an additional illustration, the wearable cardiotoxicity monitoring device 102 may filter out lower frequency recordings corresponding to respiration movements of the patient 100 from the motion signals recorded by an accelerometer, such as the accelerometer 302 or a motion sensor 528 (e.g., by applying a low-pass filter to the motion signals). The wearable cardiotoxicity monitoring device 102 may then transmit the lower frequency recordings to the remote server 104 as respiration data.

In implementations, the wearable cardiotoxicity monitoring device 102 may be similarly configured to transmit raw or minimally processed additional sensor data to the remote server 104, or perform at least some processing and/or analysis of the additional sensor data and transmit the results of the processed and/or analyzed additional sensor data. As an example, the wearable cardiotoxicity monitoring device 102 may filter out higher frequency recordings corresponding to patient activity from the motion signals recorded by an accelerometer, such as the accelerometer 302 or by a motion sensor 528 (e.g., by applying a high-pass filter to the motion signals). The wearable cardiotoxicity monitoring device 102 may then transmit the higher frequency recordings to the remote server 104 as patient motion or activity level data. As another example, the data recorded by the accelerometer 302 or motion sensor 528 may include recordings based on forces exerted by gravity (e.g., as the patient 100 changes position). The wearable cardiotoxicity monitoring device 102 may measure the amount of acceleration at any given time in the accelerometer data that is due to gravity and remove that amount of acceleration from the motion signals to generate data indicative of the body posture of the patient 100 over time. The wearable cardiotoxicity monitoring device 102 may transmit this body posture data to the remote server 104.

In implementations, any of the processing and/or analysis of the sensed biosignals and/or additional signals described above as being performed by the wearable cardiotoxicity monitoring device 102 may instead be performed by the remote server 104. Alternatively, or additionally, the wearable cardiotoxicity monitoring device 102 (e.g., at the cardiotoxicity monitoring unit 106, the portable gateway 110, the connection pod 408, and/or the medical device controller 406, depending on the implementation) may perform additional processing and/or analysis of the sensed biosignals and/or additional signals. For instance, the wearable cardiotoxicity monitoring device 102 may perform at least some of the processes described below with reference to steps 608, 610, and/or 612 and transmit the results of those processes to the remote server 104 as the biosignal-based data.

The remote server 104 receives the biosignal-based data from the wearable cardiotoxicity monitoring device 102 at step 606. For example, in implementations including the cardiotoxicity monitoring unit 106 in communication with the portable gateway 110, the remote server 104 may receive the biosignal-based data from the portable gateway 110. As another example, the remote server 104 may receive the biosignal-based data from the cardiotoxicity monitoring unit 106. As another example, in implementations including the garment-based monitoring device 118, the remote server 104 may receive the biosignal-based data directly from the garment-based monitoring device 118 (e.g., via the network interface 508 of the medical device controller 500) or through an intermediary device (e.g., implemented similarly to the portable gateway 110). In implementations, the wearable cardiotoxicity monitoring device 102 may be configured as a different embodiment from the embodiments shown and described above with reference to FIGS. 1-8, and the remote server 104 may receive the biosignal-based data from other communications circuitry of the wearable cardiotoxicity monitoring device 102. In implementations, the remote server 104 may also receive additional sensor data from the wearable cardiotoxicity monitoring device 102, such as non-biosignal sensor data, as discussed above.

The remote server 104 analyzes the biosignal-based data to identify at least one cardiotoxicity biosignal marker associated with heart failure caused by chemotherapy cardiotoxicity and/or radiation therapy cardiotoxicity (e.g., depending on the patient's oncology treatment regime) at step 608. In implementations, the remote server 104 may identify a cardiotoxicity biosignal marker directly from the biosignal-based data received from the wearable cardiotoxicity monitoring device 102. For example, the wearable cardiotoxicity monitoring device 102 may determine a thoracic fluid level, which the wearable cardiotoxicity monitoring device 102 transmits to the remote server 104. The remote server 104 may receive the determined thoracic fluid level and identify the received thoracic fluid level as a cardiotoxicity biosignal marker. In implementations, the remote server 104 may process and/or analyze the received biosignal-based data from the wearable cardiotoxicity monitoring device 102 to identify a cardiotoxicity biosignal marker. As an illustration, the remote server 104 may perform any of the processing and/or analysis described above as being performed by the wearable cardiotoxicity monitoring device 102. As another illustration, the remote server 104 may identify one or more biomarkers by sampling the biosignal-based data (e.g., as received from the wearable cardiotoxicity monitoring device 102 or after at least some processing of the biosignal-based data, such as processing to remove noise and artifacts). For instance, the remote server 104 may sample a sensed biosignal received from the wearable cardiotoxicity monitoring device 102 at a predetermined frequency as part of analyzing the biosignal-based data to identify the at least one cardiotoxicity biosignal marker. In examples, the sampled biosignal-based data may represent at least one cardiotoxicity biosignal marker, such as a series of cardiotoxicity biosignal markers (e.g., a series of cardiotoxicity biosignal markers over time). In examples, the remote server 104 may further process the sampled biosignal-based data to determine the at least one cardiotoxicity biosignal marker, such as by using the biosignal-based data to calculate a biosignal-based metric for the patient over time (e.g., the patient's heart rate, activity level, body position, etc.).

The remote server 104 then determines a current status of cardiotoxicity in the patient 100 based on the at least one cardiotoxicity marker at step 610. In implementations, the remote server 104 may compare the at least one cardiotoxicity marker to at least one predetermined threshold to determine the current status of cardiotoxicity in the patient 100. Each predetermined threshold may be associated with a risk of cardiotoxicity and/or a level of cardiotoxicity in the patient 100. As such, the remote server 104 may determine if the at least one cardiotoxicity marker transgresses the at least one predetermined threshold (e.g., is less than or equal to, or greater than or equal to, depending on the predetermined threshold). For example, the remote server 104 may identify an array or series of cardiotoxicity biomarkers and compare each of the cardiotoxicity biomarkers to an associated predetermined threshold (e.g., a first cardiotoxicity biomarker to a first predetermined threshold, a second cardiotoxicity biomarker to a second predetermined threshold, etc.) to determine whether each cardiotoxicity biomarker transgresses its associated predetermined threshold. As another example, the remote server 104 may compare a given cardiotoxicity biomarker to multiple predetermined thresholds. To illustrate, the remote server 104 may determine whether a given cardiotoxicity biomarker transgresses a first predetermined threshold associated with a first, lower heart failure severity and whether the given cardiotoxicity biomarker transgresses a second predetermined threshold associated with a second, higher heart failure severity. As another illustration, the remote server 104 may determine whether a given cardiotoxicity biomarker transgresses a first predetermined threshold associated with a first, lower level of risk for cardiotoxicity and whether the given cardiotoxicity biomarker transgresses a second predetermined threshold associated with a second, higher level of risk for cardiotoxicity.

In implementations, the remote server 104 may determine the current status of cardiotoxicity in the patient 100 using a function developed to identify a current cardiotoxicity level, current cardiotoxicity risk level, etc. for oncology patients. The at least one cardiotoxicity biomarker may thus be an input to this cardiotoxicity evaluation function. The cardiotoxicity evaluation function may be a linear function, an n-th order polynomial function, a logarithmic function, an exponential function, a power function, and/or the like, as well as combinations of these functions. Additionally, the cardiotoxicity evaluation function may have been developed, for instance, using clinical data on outcomes, risk factors, etc. for oncology patients undergoing chemotherapy and/or radiation therapy. Examples of cardiotoxicity evaluation functions are shown below, where the X values represent inputs to the cardiotoxicity evaluation function (e.g., the at least one cardiotoxicity biomarker) and the A, B, C, etc. values represent predetermined constants of the cardiotoxicity evaluation function:

$$Cardiotoxicity status = A * X + B$$

$$Cardiotoxicity status = {A}_{1} * {X}_{1} + {A}_{2} * {X}_{2} + B$$

$$Cardiotoxicity status = {A}_{1} * {X}_{1} + {A}_{2} * {X}_{2} + … + {A}_{n} * {X}_{n} + B$$

$$Cardiotoxicity status = {A}_{1} * {X}_{1} + {A}_{2} * {X}_{2} - B * {X}_{3} + C$$

$$Cardiotoxicity status = A * {X}^{B} + C$$

$$Cardiotoxicity status = {A}_{1} * {X}_{1}^{{B}_{1}} + {A}_{2} * {X}_{2}^{{B}_{2}} + C$$

$$Cardiotoxicity status = {A}_{1} * {X}_{1}^{{B}_{1}} + {A}_{2} * {X}_{2}^{{B}_{2}} + … + {A}_{n} * {X}_{n}^{{B}_{n}} + C$$

$$Cardiotoxicity status = A * log ( X ) + B$$

$$Cardiotoxicity status = {A}_{1} * log ( {X}_{1} ) + {A}_{2} * log ( {X}_{2} ) + … + {A}_{n} * log ( {X}_{n} ) + B$$

$$Cardiotoxicity status = A * {e}^{X} + C$$

$$Cardiotoxicity status = A * {e}^{B * X} + C$$

$$Cardiotoxicity status = A * {e}^{{B}_{1} * {X}_{1} + {B}_{2} * {X}_{2} + … + {B}_{n} * {X}_{n}} + C$$

In implementations, determining the current status of cardiotoxicity in the patient 100 based on the at least one cardiotoxicity biosignal marker may include determining a representative cardiotoxicity biosignal marker that the remote server 104, in turn, then uses to determine the current status of cardiotoxicity in the patient 100. For example, determining the representative cardiotoxicity biosignal marker may include applying a statistical analysis to the at least one cardiotoxicity biosignal marker. As part of the statistical analysis, the remote server 104 may determine at least one of a mean, a median, a mode, a highest value, a lowest value, and/or the like of a series of cardiotoxicity biomarker values, such as a sampled series of cardiotoxicity biomarkers or cardiotoxicity biomarkers provided over time.

In implementations, as discussed above, the biosignal-based data includes (or is processable to produce) an ECG of the patient 100. The remote server 104 may then analyze the patient's ECG to identify at least one ECG marker associated with heart failure caused by chemotherapy cardiotoxicity and/or radiation therapy cardiotoxicity as part of process 600 of FIG. 9. As an illustration, PR interval duration may be associated with cardiotoxicity in oncology patients. Administration of chemotherapy and/or radiation therapy to a patient 100 may damage the patient's heart tissues, creating a block of the patient's heart that partially or completely interrupts transmission of electrical signals across the heart. For instance, the patient 100 may suffer an atrioventricular (AV) block when signals traveling between the patient's atria and ventricles are interrupted. In various cases, chemotherapy and/or radiation therapy may cause patients to develop a heart block by exacerbating pre-existing heart conditions or by causing the damage as a side effect, such as by creating fibrosis in the heart's conduction systems. With the disruption of electrical signal transmission, the patient's heart may beat improperly. To illustrate, the patient 100 may eventually go into bradycardia. For example, studies on the chemotherapeutic drug paclitaxel have shown that patients being administered paclitaxel may experience asymptomatic sinus bradycardia, heart block, and/or other conduction abnormalities in the heart. As another example, thalidomide has also been associated with bradycardia and heart block in oncology patients. In some cases, prescribing physicians may need to react to severe heart blocks caused by chemotherapy and/or radiation therapy. Bradycardia, along with other physiological symptoms such as hemodynamic or AV conduction issues, may cause the prescribing physician to decrease the chemotherapeutic dose or change to another therapy. If no other type of therapy is available, the patient may even need to be implanted with a permanent pacemaker. The interrupted transmission of electrical signals in the patient's heart, such as those resulting in a heart block like AV block, may be reflected in the lengthening of the patient's PR intervals. Thus the patient's PR intervals may be a biosignal marker associated with cardiotoxicity and heart failure.

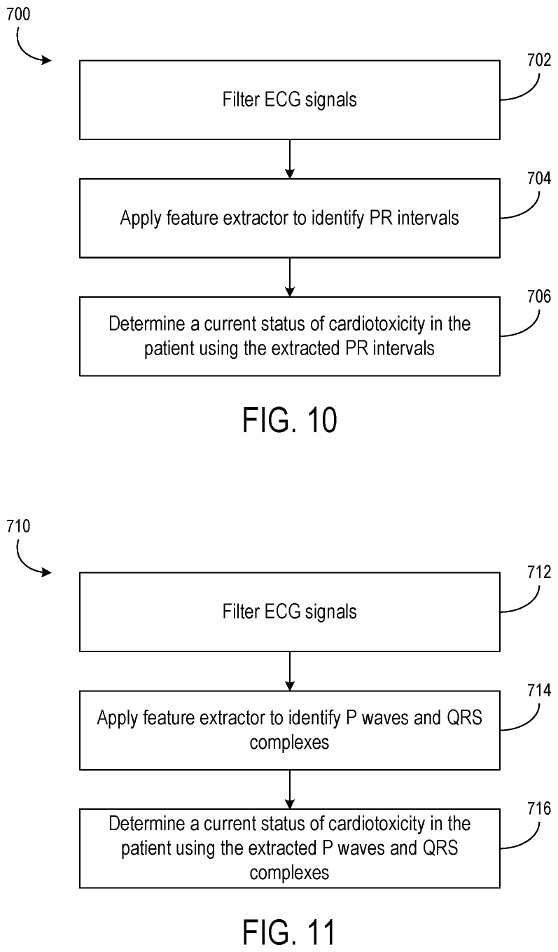

Accordingly, FIG. 10 illustrates a sample process flow for using a patient's PR intervals to determine the current status of cardiotoxicity in the patient 100 (e.g., performing steps 608 and 610 of FIG. 9, where the at least one cardiotoxicity biosignal marker includes PR intervals). The sample process 700 shown in FIG. 10 can be implemented by the remote server 104, though in some implementations, the sample process 700 may be additionally or alternatively performed by the wearable cardiotoxicity monitoring device 102 being used by the patient 100. The remote server 104 filters the ECG signals received from the wearable cardiotoxicity monitoring device 102, or generated at the remote server 104 from the biosignal-based data received from the wearable cardiotoxicity monitoring device 102, at step 702. For example, in implementations, the remote server 104 may receive raw data sampled at 250 Hz. The remote server may then remove baseline wander, high frequency noise, and 50/60 Hz interference. This filtering may be represented mathematically by denoting the raw ECG data as x and representing the process as:

$${y}_{1} = bpf * x$$

In this equation, bpf represents band pass filtering, * stands for convolution, and y₁ represents the filtered signal.

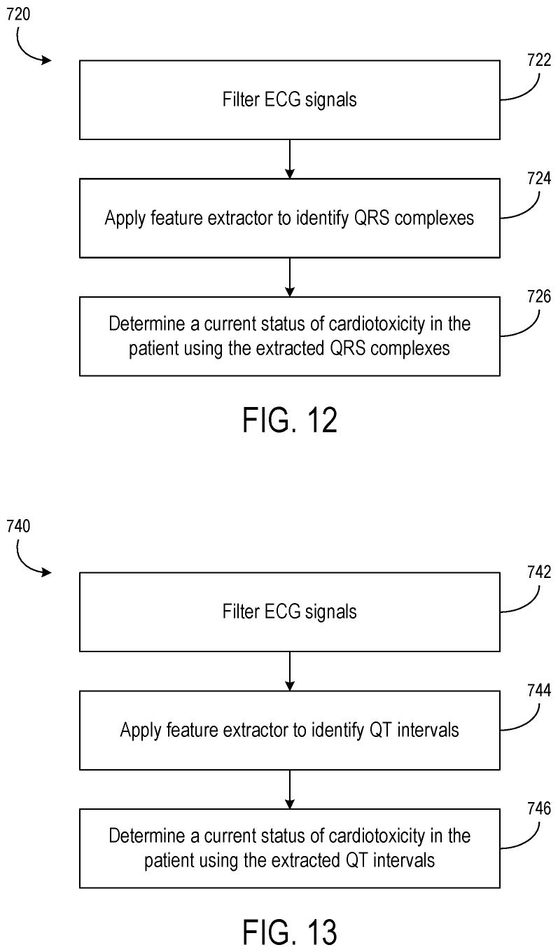

The remote server 104 then applies a feature extractor to the patient's ECG signals to identify PR intervals at step 704. For example, the remote server 104 may implement a rhythm classifier stored in a non-transitory computer readable medium (e.g., a memory, a programmable circuit board, a field programmable gate array, an integrated circuit, any combination thereof, and/or the like). The rhythm classifier may include at least one neural network trained based on a historical collection of ECG signal portions with known rhythm information. Among the rhythm information the rhythm classifier is trained to identify may be PR intervals. Additionally, the remote server 104 may detect time data corresponding to the PR intervals. In implementations, the remote server 104 may use a Pan-Tomkins-based QRS detector as part of identifying the PR intervals (e.g., described in more detail below with respect to FIG. 12). In implementations, the remote server 104 may use a Hilbert transform process to perform the PR interval detection. In implementations, the remote server 104 may use a phasor transform process to perform the PR interval detection. In implementations, the rhythm classifier may determine a confidence score associated with the detected PR intervals (e.g., output a confidence score associated with the probability that the detected PR intervals were identified correctly).

The remote server 104 determines a current status of cardiotoxicity in the patient 100 using the extracted PR intervals at step 706. In implementations, the remote server 104 may determine a series of PR intervals for the patient 100. For instance, the remote server 104 may identify all of the PR intervals for the patient 100 in a segment of the patient's ECG. As another example, the remote server 104 may randomly or periodically sample the patient's ECG signal and determine PR intervals for all of the sampled portions of the patient's ECG signal. After determining a series of PR intervals for the patient 100, the remote server 104 may compare each of the PR intervals to one or more predetermined thresholds to determine the current status of cardiotoxicity in the patient. In implementations, the remote server 104 may determine a representative PR interval for the patient 100 (e.g., from a series of PR intervals determined for the patient 100, such as by the processes just described). The representative PR interval may be, for example, a mean, median, mode, highest value, lowest value, etc. of the series of PR intervals. The remote server 104 may then compare the representative PR interval to one or more predetermined thresholds to determine the current status of cardiotoxicity in the patient 100.

To illustrate, a normal PR interval may be in a range of 120 ms to 200 ms. Accordingly, PR intervals greater than 200 ms may be indicative of AV block in the patient 100. Thus, as an example, the remote server 104 may determine that a PR interval greater than a predetermined threshold of 200 ms is associated with potential AV block in the patient 100. As another example, the remote server 104 may determine that a PR interval greater than a predetermined percentage of the 200 ms threshold is associated with potential AV block in the patient 100, such as 10% more than the predetermined threshold (e.g., more than 220 ms), 15% more than the predetermined threshold (e.g., more than 230 ms), 30% more than the predetermined threshold (e.g., more than 240 ms), and/or the like. As another example, according to the “2022 ESC Guidelines on Cardio-Oncology Developed in Collaboration with the European Hematology Association (EHA), the European Society for Therapeutic Radiology and Oncology (ESTRO) and the International Cardio-Oncology Society (IC-OS)” published by Alexander R. Lyon et al. of the European Society of Cardiology (ESC) in 43 European Heart Journal 4229 (2022) (“2022 ESC Guidelines on Cardio-Oncology”), a PR interval greater than 300 ms may be associated with advanced conduction disease in the heart. Accordingly, the remote server 104 may determine that a PR interval greater than a predetermined threshold of 300 ms is associated with potential AV block in the patient 100. As another example, the remote server 104 may determine that a PR interval is associated with one of a predetermined number of risk levels based on comparing the PR interval to a number of predetermined thresholds (e.g., 200 ms, 300 ms, etc.).

For instance, in implementations, the remote server 104 may operate according to Table 1 below, which lists various PR interval durations, along with associated risk levels and actions taken by the remote server 104 in response to finding that the patient's PR interval falls into the corresponding risk level (e.g., as part of step 612 of FIG. 9, discussed in further detail below). As shown in Table 1, if the patient's PR interval is less than or equal to 200 ms, the remote server 104 determines that the patient 100 is not at risk and takes no actions. If the patient's PR interval is greater than 200 ms and less than or equal to 250 ms, the remote server 104 determines that the patient 100 is at a medium risk of having an AV block. The remote server 104 thus flags the patient 100 and the patient's associated ECG data for the patient's physician to review (e.g., on a portal displaying information about a number of patients being overseen by the physician, where each of the patients is using a wearable cardiotoxicity monitoring device 102). The associated ECG data may include, for example, the ECG segments containing the evaluated PR intervals, the average or median PR interval duration over those ECG segments, and/or the like. In examples, the remote server 104 may also display contextual information for the physician, such as an activity level of the patient 100 for the evaluated ECG segments, whether the patient 100 was sleeping at the time the evaluated ECG segments were recorded, a body posture of the patient 100 at the time the evaluated ECG segments were recorded, and/or the like. The remote server 104 also highlights the patient 100 in yellow for the physician so that the physician can quickly evaluate the risk of the various patients, including the patient 100, being overseen by the physician. If the patient's PR interval is greater than 250 ms and less than or equal to 300 ms, the remote server 104 determines that the patient 100 is at a high risk of having an AV block. The remote server 104 flags the patient 100 and the patient's associated ECG data for the patient's physician to review in orange. Finally, if the patient's PR interval is greater than 300 ms, the remote server 104, determines that the patient is at a very high risk of having an AV block. The remote server 104 flags the patient 100 and the patient's associated ECG data in red. The remote server 104 also sends an immediate alert to the patient's physician, such as by sending the patient's physician an email alerting the physician of the patient's risk level and requesting that the physician review the patient's ECG data on the information portal.

TABLE 1
Example Risk Levels and Actions Taken for PR Interval Durations

| PR Interval | Risk | Action |
|---|---|---|
| Duration | Level | Taken |
| PR interval ≤ | Green | No action taken |
| 200 ms | (No risk) | |
| 200 ms < PR | Yellow | Flag the patient and the patient's |
| interval ≤ | (Medium) | associated ECG data for the |
| 250 ms | | patient's physician to review |
| | | Highlight the associated patient |
| | | data in yellow |
| 250 ms < PR | Orange | Flag the patient and the patient's |
| interval ≤ | (High) | associated ECG data for the |
| 300 ms | | patient's physician to review |
| | | Highlight the associated patient |
| | | data in orange |
| PR interval > | Red | Flag the patient and the patient's |
| 300 ms | (Very High) | associated ECG data for the |
| | | patient's physician to review |
| | | Highlight the associated patient |
| | | data in red |
| | | Send an immediate alert to the |
| | | patient's physician |

Table 1 is an example of predetermined thresholds, associated risk levels, and actions that a remote server 104 may use. In other examples, the remote server 104 may use other and/or additional predetermined thresholds, associated risk levels, and/or actions as part of implementing process 700 of FIG. 10. Additionally, in implementations, the remote server 104 may apply Table 1 or a similar decision-making tree using a representative PR interval for the patient 100. In implementations, the remote server 104 may apply Table 1 or a similar decision-making tree according to how many or what percentage of a series of PR intervals fall into the categories shown in Table 1 or in a similar decision-making tree. For example, the remote server 104 may determine that the patient's PR intervals overall (e.g., at least 50%, at least 60%, at least 75%, etc.) fall into the risk category matching the plurality of PR intervals. As another example, the remote server 104 may determine that, if at least a predetermined percentage of the patient's PR intervals fall into one or more of the yellow, orange, and red risk categories (e.g., at least 20% of the patient's PR intervals, at least 25% of the patient's PR intervals, at least 30% of the patient's PR intervals, etc.), the patient 100 should be assigned the corresponding highest risk category that the patient 100 has at least the predetermined percentage of PR intervals falling into.

As another illustration, loss of 1:1 conduction may be associated with cardiotoxicity in oncology patients as another indicator of heart block. In various cases, elongated PR intervals, as discussed above, may be indicative of first-degree heart block, where electrical signals in the heart are still traveling to the ventricles, albeit more slowly than in a healthy heart. However, patients with more severe heart block may experience the loss of 1:1 conduction, or the loss of electrical signals equally reaching the atria and the ventricles. Depending on the level of heart block, electrical signals may periodically (e.g., with second-degree heart block) to frequently or continuously (e.g., with third-degree heart block) be stopped from reaching the ventricles. For patients without 1:1 conduction, the loss of 1:1 conduction may be reflected in the patient's ECG signals. More specifically, the patient may have an ECG signal where in one or more cardiac cycles, a P wave is not followed by a QRS complex, showing that the electrical signals were blocked from reaching the patient's ventricles for the cardiac cycle.

FIG. 11 thus illustrates a sample process flow for using a patient's P waves and QRS complexes to determine the current status of cardiotoxicity in the patient 100 (e.g., performing steps of 608 and 610 of FIG. 9, where the at least one cardiotoxicity biosignal marker includes P waves and QRS complexes). The sample process 710 shown in FIG. 11 can be implemented by the remote server 104, though in some implementations, the sample process 710 may be additionally or alternatively performed by the wearable cardiotoxicity monitoring device 102 being used by the patient 100. The remote server 104 filters the ECG signals received from the wearable cardiotoxicity monitoring device 102, or generated at the remote server 104 from the biosignal-based data received from the wearable cardiotoxicity monitoring device 102, at step 712. In implementations, the remote server 104 may perform the process of step 712 similarly to the process of step 702, described above with reference to FIG. 10. The remote server 104 applies a feature extractor to the patient's ECG signals to identify P waves and QRS complexes at step 714. In implementations, the remote server 104 may perform the process of step 714 similarly to the process of step 704, described above with reference to FIG. 10. In implementations, the remote server 104 may identify the QRS complexes at step 714 similarly to the processes described below with respect to step 724 of FIG. 12.

The remote server 104 determines a current status of cardiotoxicity in the patient 100 using the extracted P waves and QRS complexes at step 716. For example, the remote server 104 may determine in a given ECG segment (e.g., a one-minute segment, a two-minute segment, a five-minute segment, a ten-minute segment, etc.) whether each cardiac cycle includes a P wave followed by a QRS complex. If each P wave or most P waves (e.g., 99% of P waves in the ECG segment) are followed by a QRS complex in the ECG segment, the remote server 104 may determine that the patient 100 is showing healthy 1:1 conduction. If at least a predetermined number or percentage of P waves in the ECG segment are not followed by a QRS complex (e.g., 1% of P waves, 5% of P waves, 10% of P waves, 15% of P waves, 20% of P waves, etc.), the remote server 104 may determine that the patient 100 is likely suffering from a loss of 1:1 conduction.

For instance, in implementations, the remote server 104 may operate according to Table 2 below. Table 2 lists various percentages of P waves in an ECG segment not followed by a QRS complex, along with associated risk levels and actions taken by the remote server 104 in response to finding that the patient's ECG segment falls into the corresponding risk level (e.g., as part of step 612 of FIG. 9, discussed in further detail below). Table 2 is structured similarly to Table 1 above, and in various embodiments, the remote server 104 may implement Table 2 similarly to the implementation of Table 1 discussed above. As shown in Table 2, if less than 1% of cardiac cycles within an ECG segment contain a P wave not followed by a QRS complex, the remote server 104 determines that the patient 100 is not at risk and takes no actions. If between 1% and 10% of cardiac cycles within the ECG segment contain a P wave not followed by a QRS complex, the remote server 104 determines that the patient 100 is at medium risk due to some loss of 1:1 conductivity. The remote server 104 flags the patient 100 and the patient's associated ECG data for the patient's physician to review. The remote server 104 also highlights the patient 100 in yellow for the physician so that the physician can quickly evaluate the risk of various patients, including patient 100, being overseen by the physician. If more than 10% of cardiac cycles within the ECG segment contain a P wave not followed by a QRS complex, the remote server 104 determines that the patient is at high risk due to significant loss of 1:1 conductivity. The remote server 104 flags the patient 100 and the patient's associated ECG data in red and sends an immediate alert to the patient's physician.

TABLE 2
Example Risk Levels and Actions Taken
for Loss of 1:1 Conductivity

| Cardiac Cycles with a P Wave | | |
|---|---|---|
| Not Followed by | Risk | Action |
| a QRS Complex | Level | Taken |
| Less than 1% of cardiac | Green | No action taken |
| cycles within ECG segment | (No risk) | |
| 1% to 10% of cardiac | Yellow | Flag the patient and the |
| cycles within | (Medium) | patient's associated ECG |
| ECG segment | | data for the patient's |
| | | physician to review |
| | | Highlight the associated |
| | | patient data in yellow |
| Greater than 10% | Red | Flag the patient and the |
| of cardiac cycles | (High) | patient's associated ECG |
| within ECG segment | | data for the patient's |
| | | physician to review |
| | | Highlight the associated |
| | | patient data in red |
| | | Send an immediate alert to |
| | | the patient's physician |

Table 2 is an example of predetermined thresholds, associated risk levels, and actions that a remote server 104 may use. In other examples, the remote server 104 may use other and/or additional predetermined thresholds, associated risk levels, and/or actions as part of implementing process 710 of FIG. 11. Additionally, in implementations, the remote server 104 may apply Table 2 or a similar decision-making tree using a representative ECG segment, such as a randomly sampled ECG segment, or using a number of ECG segments, such as 10 segments, 20 segments, 30 segments, 40 segments, 50 segments, etc., over a 24 hour period, 48, hour period, 72 hour period, and/or the like.

As another illustration, rather than separating PR interval prolongation and 1:1 conduction metrics, the remote server 104 may combine the PR interval prolongation and 1:1 conduction metrics to determine an overall heart block status of the patient 100. For example, the remote server 104 may determine that the patient 100 has first-degree heart block when their PR interval is more than a predetermined threshold, such as 300 ms. The remote server 104 may determine that the patient 100 has second-degree heart block when a predetermined percentage of their cardiac cycles include a P wave not followed by a QRS complex, such as 5% or 10%. The remote server 104 may flag a patient 100 with first-degree heart block in yellow and flag a patient 100 with second-degree heart block in red.

As another illustration, QRS complex duration may be associated with cardiotoxicity in oncology patients. Administration of chemotherapy and/or radiation therapy to the patient 100 may damage the patient's heart such that the heart has more difficulty in pumping blood. Various studies on chemotherapeutic agents have found that patients may suffer heart failure at rates of at least 1-5% and asymptomatic reduction in left ventricular function at rates of at least 5-20%. For example, prescribing physicians may need to monitor the patient's left ventricular ejection fraction (LVEF), both during and after administration of chemotherapy and/or radiation therapy to the patient. As an illustration, patients receiving trastruzumab may need regular LVEF monitoring while the patient is receiving treatment (e.g., every three months) and after treatment has been completed (e.g., every six months for two years after treatment). If the patients start experiencing a certain level of LVEF reduction, such as 16% from a baseline or less than 55% overall, the prescribing physician may instruct the patient to refrain from taking further doses until their LVEF recovers or refer the patient to a cardiologist for heart failure treatment if their LVEF does not recover. An elongated QRS complex duration may be indicative that electrical signals are taking longer to move across the patient's heart and activate the ventricles. This longer QRS complex duration may in turn be associated with ventricular dysfunction, such as LVEF reduction.

As such, FIG. 12 illustrates a sample process flow for using a patient's QRS complex durations to determine the current status of cardiotoxicity in the patient 100 (e.g., performing steps 608 and 610 of FIG. 9, where the at least one cardiotoxicity biosignal marker includes QRS complexes). The sample process 720 shown in FIG. 12 can be implemented by the remote server 104, though in some implementations, the sample process 720 may additionally or alternatively be performed by the wearable cardiotoxicity monitoring device 102 being used by the patient 100. The remote server 104 filters the ECG signals received from the wearable cardiotoxicity monitoring device 102, or generated at the remote server 104 from the biosignal-based data received from the wearable cardiotoxicity monitoring device 102, at step 722. In implementations, the remote server 104 may perform the process of step 722 similarly to the process of step 702, described above with reference to FIG. 10. The remote server 104 applies a feature extractor to the patient's ECG signals to identify QRS complexes at step 724. In implementations, the remote server 104 may perform the process of step 724 similarly to the process of step 704, described above with reference to FIG. 10. For example, in implementations, the remote server 104 may use a Pan-Tompkins-based QRS detector. To illustrate, the remote server 104 may find the derivative of the filtered y₁ signal shown above with reference to FIG. 7 and square the result, which may be represented as:

$${y}_{2} = {( \frac{{dy}_{1}}{dt} )}^{2}$$

Next, the remote server 104 applies a moving average to the y₂ result, which may be further represented mathematically by:

$${y}_{3} = MovingAverageFilter * {y}_{2}$$

The remote server 104 then applies an adaptive power threshold to locate the QRS complexes in the y₃ signal.

The remote server 104 determines a current status of cardiotoxicity in the patient 100 using the extracted QRS complexes at step 726. In implementations, the remote server 104 may determine the current status of cardiotoxicity in the patient 100 using the extracted QRS complexes similarly to how the remote server 104 may determine the current status of cardiotoxicity in the patient 100 using the extracted PR intervals at step 706 of FIG. 10. For example, the remote server 104 may determine a series of QRS complexes for the patient 100, and/or the remote server 104 may determine a representative QRS complex for the patient 100. The remote server 104 may then compare the series of QRS complexes and/or the representative QRS complex to one or more predetermined thresholds to determine the current status of cardiotoxicity in the patient 100.

As an illustration, a normal QRS complex duration may last 80 to 100 ms. Thus, QRS complexes lasting more than 100 ms may be indicative of conductivity issues and/or weakened ventricular contractions in the patient 100. For example, the remote server 104 may determine that a QRS complex lasting more than a predetermined threshold of 100 ms may be indicative of conductivity issues and/or weakened ventricular contractions in the patient 100. As another example, the remote server 104 may determine that a QRS complex lasting more than a predetermined percentage of the 100 ms threshold is associated with potential conductivity issues and/or weakened ventricular contractions in the patient 100. Examples of the predetermined percentage are 5% more than the predetermined threshold (e.g., more than 105 ms), 10% ms more than the predetermined threshold (e.g., more than 110 ms), 15% more than the predetermined threshold (e.g., more than 115 ms), and/or the like. As another example, a QRS complex lasting 120 ms or more may be associated with heart failure. Accordingly, the remote server 104 may determine that a QRS complex lasting longer than a predetermined threshold of 120 ms is indicative of conductivity issues and/or weakened ventricular contractions in the patient 100. As another example, the remote server 104 may determine whether a QRS complex is associated with one of a predetermined number of risk levels based on comparing the QRS complex duration to a number of predetermined thresholds (e.g., 100 ms, 120 ms, etc.).

For instance, in implementations, the remote server 104 may operate according to Table 3 below, which lists various QRS complex durations, along with associated risk levels and actions taken by the remote server 104 in response to finding that the patient's QRS complex falls into the corresponding risk level (e.g., as part of step 612 of FIG. 9, discussed in further detail below). Table 3 is structured similarly to Tables 1 and 2 above, and in various embodiments, the remote server 104 may implement Table 3 similarly to the implementation of Tables 1 and 2 discussed above. As shown in Table 3, if the patient's QRS complex duration is less than or equal to 100 ms, the remote server 104 determines that the patient 100 is not at risk and takes no actions. If the patient's QRS complex duration is greater than 100 ms and less than 120 ms, the remote server 104 determines that the patient 100 is at medium risk of having conductivity issues and/or weakened ventricular contractions. The remote server 104 thus flags the patient 100 and the patient's associated ECG data for the patient's physician to review. The remote server 104 also highlights the patient 100 in yellow for the physician so that the physician can quickly evaluate the risk of various patients, including patient 100, being overseen by the physician. If the patient's QRS complex duration is greater than or equal to 120 ms, the remote server 104 determines that the patient 100 is at a high risk of having conductivity issues and/or weakened ventricular contractions. The remote server 104 flags the patient 100 and the patient's associated ECG data in red. The remote server 104 also sends an immediate alert to the patient's physician.

TABLE 3
Example Risk Levels and Actions
Taken for QRS Complex Durations

| QRS Complex | Risk | Action |
|---|---|---|
| Duration | Level | Taken |
| QRS complex ≤ | Green | No action taken |
| 100 ms | (No risk) | |
| 100 ms < QRS | Yellow | Flag the patient and the |
| complex < 120 ms | (Medium) | patient's associated ECG data |
| | | for the patient's physician to |
| | | review |
| | | Highlight the associated |
| | | patient data in yellow |
| QRS complex ≥ | Red | Flag the patient and the |
| 120 ms | (High) | patient's associated ECG data |
| | | for the patient's physician to |
| | | review |
| | | Highlight the associated |
| | | patient data in red |
| | | Send an immediate alert to the |
| | | patient's physician |

Table 3 is an example of predetermined thresholds, associated risk levels, and actions that a remote server 104 may use. In other examples, the remote server 104 may use other and/or additional predetermined thresholds, associated risk levels, and/or actions as part of implementing process 720 of FIG. 12. Additionally, in implementations and similar to Tables 1 and 2 described above, the remote server 104 may apply Table 3 or a similar decision-making tree using a representative QRS complex for the patient 100. In implementations, the remote server 104 may apply Table 3 or a similar decision-making tree according to how many or what percentage of a series of QRS complexes fall into the categories shown in Table 3 or a similar decision-making tree.

Alternatively, or additionally, the remote server 104 may use QRS complex morphology to determine the current status of cardiotoxicity in the patient 100 at step 726. For example, deviations from typical QRS complex morphology may also be illustrative of electrical signals taking longer to move across the patient's heart and activate the ventricles. As such, the remote server 104 may compare the patient's QRS complex morphology to a reference or baseline QRS complex morphology to determine whether the patient's QRS complex shows significant deviations from the reference or baseline QRS complex morphology.

In implementations, for example, the patient 100 may use the wearable cardiotoxicity monitoring device 102 before the patient 100 begins a chemotherapy and/or radiation therapy treatment regime, where the wearable cardiotoxicity monitoring device 102 generates baseline ECG signals for the patient 100. The wearable cardiotoxicity monitoring device 102 transmits the baseline ECG signals to the remote server 104, and the remote server 104 uses the baseline ECG signals to create a baseline ECG segment for the patient 100 (e.g., including a baseline P wave, baseline QRS complex, and baseline T wave). For instance, the remote server 104 may use a feature extractor to identify individual P waves, QRS complexes, and T waves for the patient 100 from the patient's ECG signals (e.g., using similar feature extractor processes to the processes described above with reference to step 704 of FIG. 10). The remote server 104 may then average the extracted features to create a baseline cardiac cycle for the patient 100. When the patient 100 subsequently uses the wearable cardiotoxicity monitoring device 102 during chemotherapy and/or radiation therapy treatment, the remote server 104 may compare the patient's QRS complexes to the patient's baseline QRS complex to determine an amount of deviation from the baseline QRS complex. For each QRS complex, the amount of deviation may be output, for example, as a percentage of deviation from the baseline QRS complex based on the points in the current QRS complex and the baseline QRS complex being compared. The remote server 104 may then produce a representative statistic of the deviations from the baseline for the QRS complexes, such as an average deviation from the QRS complex, along with a standard deviation for the average. Alternatively, the remote server 104 may compare a representative QRS complex to the baseline QRS complex. As an example, the remote server 104 may average a predetermined number of QRS complexes or all of the QRS complexes in an ECG segment to output a representative QRS complex for the patient 100 that the remote server 104 then compares to the baseline QRS complex. In implementations, instead of or in addition to using a baseline QRS complex, the remote server 104 may use a reference QRS complex. For instance, the remote server 104 may identify a reference QRS complex for the patient 100 in a library based on demographic information for the patient 100 (e.g., the patient's age, sex, heart failure stage at the beginning of treatment, and/or the like).

The remote server 104 may determine that the patient 100 is likely suffering from heart conductivity issues based on the patient's deviations from the reference or baseline QRS complex. For example, the remote server 104 may determine that the patient 100 is likely suffering from heart conductivity issues when the patient's QRS complexes show at least a 10% deviation, 15% deviation, 20% deviation, 25% deviation, 30% deviation, 35% deviation, 40% deviation, and/or the like from the reference or baseline QRS complex. As another example, the remote server 104 may determine that the patient 100 is likely suffering from heart conductivity issues when the patient's deviation from the reference or baseline QRS complex is statistically significant (e.g., with a p-value of 0.05, 0.01, etc.). In implementations, the remote server 104 may take different actions depending on whether the patient's deviations from the reference or baseline QRS complex transgress different predetermined thresholds, similar to the discussion of Table 3 above. For instance, the remote server 104 may flag a patient's ECG information in yellow when the patient's overall percent deviation from the reference or baseline QRS complex is greater than 15% and flag a patient's ECG information in red and alert the patient's physician when the patient's overall percent deviation from the reference or baseline QRS complex is greater than 30%.

In implementations, rather than or in addition to identifying an overall deviation from the reference or baseline QRS complex, the remote server 104 may identify whether the patient's QRS complex morphology includes particular features. For example, the remote server 104 may use a feature extractor to determine whether the patient's QRS complexes (and/or a representative QRS complex) include a notch, which may be indicative of scarring of the cardiac tissues. As another example, the remote server 104 may use a feature extractor to determine whether the patient's QRS complexes (and/or a representative QRS complex) are inverted (e.g., relative to a baseline for the patient 100). As another example, the remote server 104 may use a feature extractor to determine whether a patient's QRS complexes (and/or a representative QRS complex) show large changes in amplitude (e.g., a 5% change, 10% change, 15% change, 20% change, etc. relative to a baseline for the patient 100), such as decreased amplitudes. QRS inversions and changes in amplitudes may also be indicative of conduction issues in the patient's heart. As noted above, identifying particular features in the patient's QRS complex may be done by comparing the QRS complex to a reference or baseline QRS complex. Alternatively, or additionally, identifying features in the patient's QRS complex may be independent of a reference or baseline QRS complex. For example, the remote server 104 may be able to identify notches in the patient's QRS complexes regardless of whether the QRS complexes are compared to a reference or baseline.

As another illustration, QT interval duration may be associated with cardiotoxicity in oncology patients. As discussed above, administering chemotherapy and/or radiation therapy to the patient 100 may damage the patient's heart. In some cases, heart damage may expressed as QT interval prolongation. For example, QT prolongation may be a common side effect of monoclonal antibody agents and tyrosine kinase inhibitor agents. In turn, QT interval prolongation may be considered an indicator of ventricular arrhythmia risk. Additionally, ventricular arrhythmias developed by patients with prolongated QT intervals may be more difficult to defibrillate, such as torsade de pointes (TdP). Patients experiencing QT prolongation may also be more likely to suffer sudden death and other cardiac repolarization abnormalities.

Accordingly, FIG. 13 illustrates a sample process flow for using a patient's QT interval durations to determine the current status of cardiotoxicity in the patient 100 (e.g., performing steps 608 and 610 of FIG. 9, where the at least one cardiotoxicity biosignal marker includes QT intervals). The sample process 740 shown in FIG. 13 can be implemented by the remote server 104, though in some implementations, the sample process 740 may additionally or alternatively be implemented by the wearable cardiotoxicity monitoring device 102 being used by the patient 100. The remote server 104 filters the ECG signals received from the wearable cardiotoxicity monitoring device 102, or generated at the remote server 104 from the biosignal-based data received from the wearable cardiotoxicity monitoring device 102, at step 742. In implementations, the remote server 104 may perform the process of step 742 similarly to the process of step 702, described above with reference to FIG. 10. The remote server 104 applies a feature extractor to the patient's ECG signals to identify QRS complexes at step 744. In implementations, the remote server 104 may perform the process of step 744 similarly to the process of step 704, described above with reference to FIG. 10.

The remote server 104 determines a current status of cardiotoxicity in the patient 100 using the extracted QT intervals at step 746. In implementations, the remote server 104 may determine the current status of cardiotoxicity in the patient 100 using the extracted QRS complexes similarly to how the remote server 104 may determine the current status of cardiotoxicity in the patient 100 using the extracted PR intervals at step 706 of FIG. 10 and using the QRS complexes at step 726 of FIG. 12. For example, the remote server 104 may determine a series of QT intervals for the patient 100, and/or the remote server 104 may determine a representative QT interval for the patient 100. The remote server 104 may then compare the series of QT complexes and/or the representative QT complex to one or more predetermined thresholds to determine the current status of cardiotoxicity in the patient 100.

As an illustration, a normal QT interval may be 350 to 430 ms in men or 350 to 440 ms in women. Accordingly, QT intervals lasting more than 430 ms in men and more than 440 ms in women may be indicative of cardiac repolarization issues in the patient 100. For example, the remote server 104 may determine that a QT interval lasting more than a predetermined threshold of 430 ms, if the patient 100 is male, and more than 440 ms, if the patient 100 is female, may be indicative of cardiac repolarization issues in the patient 100. As another example, the remote server 104 may determine that a QT interval lasting more than a predetermined percentage of the 430 ms or 440 ms threshold is associated with potential cardiac repolarization issues in the patient 100. Examples of such predetermines percentages are 2% more than the predetermined threshold (e.g., more than 439 ms if the patient 100 is male or more than 449 ms if the patient 100 is female), 5% more than the predetermined threshold, (e.g., more than 452 ms if the patient 100 is male or more than 462 ms if the patient is female), 8% more than the predetermined threshold (e.g., more than 464 ms if the patient 100 is male or more than 475 ms if the patient is female), 10% more than the predetermined threshold (e.g., more than 473 ms if the patient 100 is male or more than 484 ms if the patient is female), and/or the like. As another example, according to the “2022 ESC Guidelines on Cardio-Oncology,” a QT interval longer than 450 ms for men or 460 ms for women may be in the upper 99% of QT values. Thus, the remote server 104 may determine that a QT interval lasting longer than 450 ms, if the patient 100 is male, or 460 ms, if the patient 100 is female, is indicative of cardiac repolarization issues.

As another example, the remote server 104 may determine whether a patient's QT interval is associated with one of a predetermined number of risk levels based on comparing the QT interval to a number of predetermined thresholds. For instance, the “2022 ECG Guidelines on Cardio-Oncology” further notes that if a patient receiving an anticancer drug regimen has a QT interval that is greater than 480 ms (regardless of sex), the patient should undergo weekly ECG monitoring and the patient's physician should correct reversible causes. Alternatively, if the patient's QT interval is greater than or equal to 500 ms (regardless of sex), the patient should not take further anticancer drugs. Accordingly, the remote server 104 may compare the patient's QT intervals to a first predetermined threshold of 480 ms and a second predetermined threshold of 500 ms.

For example, in implementations, the remote server 104 may operate according to Table 4 below, which lists various QT interval durations, along with associated risk levels and actions taken by the remote server 104 in response to finding that the patient's QT interval falls into the corresponding risk level (e.g., as part of step 612 of FIG. 9, discussed in further detail below). Table 4 is structured similarly to Tables 1, 2, and 3 above, and in variously embodiments, the remote server 104 may implement Table 4 similarly to the implementations of Tables 1, 2, and 3 discussed above. As shown in Table 4, if the patient's QT interval duration is less than or equal to 430 ms (if the patient 100 is male) or 440 ms (if the patient 100 is female), the remote server 104 determines that the patient 100 is not at risk and takes no actions. If the patient's QT interval is greater than 430 ms and less than or equal to 450 ms (if the patient 100 is male) or greater than 440 ms and less than or equal to 460 ms (if the patient 100 is female), the remote server 104 determines that the patient 100 is at medium risk of having cardiac repolarization issues. The remote server 104 thus flags the patient 100 and the patient's associated ECG data for the patient's physician to review. The remote server 104 also highlights the patient 100 in yellow for the physician so that the physician can quickly evaluate the risk of various patients, including patient 100, being overseen by the physician. If the patient's QT interval is greater than 450 ms and less than or equal to 480 ms (if the patient 100 is male) or greater than 460 ms and less than or equal to 480 ms (if the patient 100 is female), the remote server 104 determines that the patient 100 is at high risk of having cardiac repolarization issues. The remote server 104 flags the patient 100 and the patient's associated ECG data for the patient's physician to review in orange. If the patient's QT interval is greater than 480 ms and less than or equal to 500 ms, the remote server 104 determines that the patient 100 is at a very high risk of having cardiac repolarization issues. The remote server 104 flags the patient 100 and the patient's associated ECG data in red. The remote server 104 also sends an immediate alert to the patient's physician. Finally, if the patient's QT interval is greater than 500 ms, the physician needs to take immediate action with respect to the patient's chemotherapy and/or radiation therapy treatment. The remote server 104 thus flags the patient 100 and the patient's associated ECG data in gray or black and sends an immediate alert to the patient's physician. The remote server 104 additionally sends an alert to the patient 100, such as by sending an email or a text message to the patient 100 indicating that the patient 100 needs to contact their physician to discuss their oncology treatment.

TABLE 4
Example Risk Levels and Actions Taken for QT Interval Durations

| QT Interval | Risk | Action |
|---|---|---|
| Duration | Level | Taken |
| QT interval ≤ | Green | No action taken |
| 430 ms (male) | (No risk) | |
| QT interval ≤ | | |
| 440 ms (female) | | |
| 430 ms < QT | Yellow | Flag the patient and the |
| interval ≤ 450 | (Medium) | patient's associated ECG data |
| ms (male) | | for the patient's physician to |
| 440 ms < QT | | review |
| interval ≤ 460 | | Highlight the associated patient |
| ms (female) | | data in yellow |
| 450 ms < QT | Orange | Flag the patient and the |
| interval ≤ 480 | (High) | patient's associated ECG data |
| ms (male) | | for the patient's physician to |
| 460 ms < QT | | review |
| interval ≤ 480 | | Highlight the associated patient |
| ms (female) | | data in orange |
| 480 ms < QT | Red | Flag the patient and the |
| interval ≤ | (Very High) | patient's associated ECG data |
| 500 ms | | for the patient's physician to |
| | | review |
| | | Highlight the associated patient |
| | | data in red |
| | | Send an immediate alert to the |
| | | patient's physician |
| QT interval > | Black | Flag the patient and the |
| 500 ms | (Immediate | patient's associated ECG data |
| | Action Needed) | for the patient's physician to |
| | | review |
| | | Highlight the associated patient |
| | | data in red |
| | | Send an immediate alert to the |
| | | patient's physician |
| | | Send an alert to the patient |

Table 4 is an example of predetermined thresholds, associated risk levels, and actions that a remote server 104 may use. In other examples, the remote server 104 may use other and/or additional predetermined thresholds, associated risk levels, and/or actions as part of implementing process 740 of FIG. 13. Additionally, in implementations and similar to Tables 1, 2, and 3 above, the remote server 104 may apply Table 4 or a similar decision-making tree using a representative QT interval for the patient 100. In implementations, the remote server 104 may apply Table 4 or a similar decision-making tree according to how many or what percentage of a series of QT intervals fall into the categories shown in Table 4 or a similar decision-making tree.

As another illustration, changes in T wave morphology may be associated with cardiotoxicity in oncology patients as another indicator of ventricular arrhythmia risk. In other cases, changes in T wave morphology may be associated with other conditions affecting the heart. For example, hyperkalemia, or the presence of too much potassium in the patient's blood, may manifest in T waves that are peaked and/or have a higher than normal amplitude. As another example, left or right bundle branch block in a patient's heart can be associated with inversion of the T wave. As another example, central nervous system damage can cause deep, inverted T waves (e.g., “cerebral” T waves). Accordingly, a patient's T wave morphology may be monitored to see if the patient is experiencing one of a number of cardiac or cardiac-related conditions.

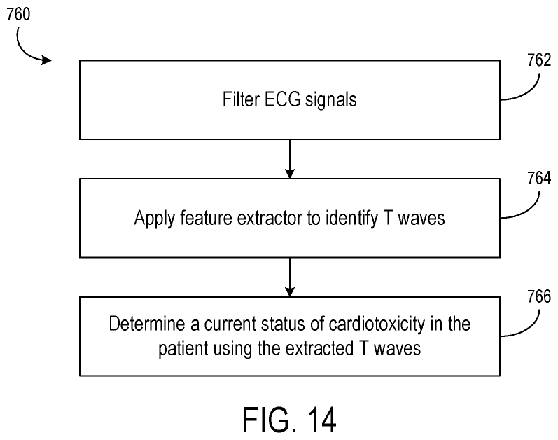

FIG. 14 illustrates a sample process flow for using a patient's T waves to determine the current status of cardiotoxicity in the patient 100 (e.g., performing steps 608 and 610 of FIG. 9, where the at least one cardiotoxicity biosignal marker includes T waves). The sample process 760 shown in FIG. 14 can be implemented by the remote server 104, though in some implementations, the sample process 760 may be additionally or alternatively performed by the wearable cardiotoxicity monitoring device 102 being used by the patient 100. The remote server 104 filters the ECG signals received from the wearable cardiotoxicity monitoring device 102, or generated at the remote server 104 from the biosignal-based data received from the wearable cardiotoxicity monitoring device 102, at step 762. In implementations, the remote server 104 may perform the process of step 762 similarly to the process of step 702, described above with reference to FIG. 10. The remote server 104 applies a feature extractor to the patient's ECG signals to identify T waves at step 764. In implementations, the remote server 104 may perform the process of step 764 similarly to the process of step 704, described above with reference to FIG. 10.

The remote server 104 determines a current status of cardiotoxicity in the patient 100 using the extracted T waves at step 766. In implementations, the remote server 104 may determine the current status of cardiotoxicity in the patient 100 using the extracted T waves similarly to how the remote server 104 may determine the current status of cardiotoxicity in the patient 100 using the morphology of the extracted QRS complexes at step 726 of FIG. 12. For example, in implementations and similar to the processes described above with respect to step 726, the patient 100 may use the wearable cardiotoxicity monitoring device 102 before the patient 100 begins a chemotherapy and/or radiation therapy treatment regime, where the wearable cardiotoxicity monitoring device 102 generates baseline ECG signals for the patient 100. The wearable cardiotoxicity monitoring device 102 transmits the baseline ECG signals to the remote server 104, and the remote server 104 uses the baseline ECG signals to create a baseline T wave for the patient 100. In implementations, instead of or in addition to using a baseline T wave, the remote server 104 may use a reference T wave.

Similar to the discussions of deviations in QRS complexes above, the remote server 104 may determine that the patient 100 is likely suffering from one or more cardiac or cardiac-related conditions based on the patient's deviations from the reference or baseline T wave at step 766. For example, the remote server 104 may determine that the patient 100 is likely suffering a cardiac or cardiac-related condition when the patient's T waves show at least a 10% deviation, 15% deviation, 20% deviation, 30% deviation, 35% deviation, 40% deviation, and/or the like from the reference or baseline T wave. As another example, the remote server 104 may determine that the patient 100 is likely suffering from a cardiac or cardiac-related condition when the patient's deviation from the reference or baseline T wave is statistically significant. In implementations, the remote server 104 may take different actions depending on whether the patient's deviations from the reference or baseline T wave transgress different predetermined thresholds, similar to the discussion of the Tables above.

In implementations, rather than or in addition to identifying an overall deviation from the reference or baseline T wave, the remote server 104 may identify whether the patient's T wave morphology includes particular features. For example, the remote server 104 may use a feature extractor to determine whether the patient's T waves are inverted (e.g., relative to a baseline recorded for the patient 100). If the patient's T waves are inverted, the remote server 104 may determine that the patient 100 is likely suffering from cardiotoxicity (e.g., a right or left bundle block) or another type of toxicity affecting the heart (e.g., central nervous system damage) from their chemotherapy and/or radiation therapy treatments. As another example, for a given cardiac cycle the remote server 104 may determine a peak amplitude of the patient's T wave and compare the peak amplitude of the T wave to the peak amplitude of the patient's QRS complex in the same cardiac cycle. If the T wave peak amplitude is within a predetermined percentage of the QRS complex peak amplitude (e.g., within 70% of the QRS complex peak amplitude, 75% of the QRS complex peak amplitude, 80% of the QRS complex peak amplitude, etc.), the remote server 104 may determine that the patient 100 is likely suffering from hyperkalemia from their chemotherapy and/or radiation therapy treatments.

In implementations, and referring back to steps 608 and 610 of FIG. 9, the remote server 104 may determine another type of cardiotoxicity biosignal marker from the patient's ECG signal, such as the patient's heart rate or heart rate recovery. For example, the remote server 104 may filter the ECG signals and identify QRS complexes in the ECG signal as discussed above with reference to steps 722 and 724 of FIG. 12. The remote server 104 may identify the R peaks in the QRS complexes and use successive R peaks to determine the patient's heart rate over a predetermined amount of time. As an illustration, the remote server 104 may determine the patient's average heart rate over a five-minute ECG strip during which the patient 100 was at rest (e.g., as determined by accelerometer counts from the accelerometer 302 or the at least one motion sensor 528) as a cardiotoxicity biosignal marker for the patient 100.

The remote server 104 may then use the patient's resting heart rate to determine the current cardiotoxicity status for the patient 100 (e.g., as part of performing step 610 of FIG. 9). In examples, the remote server 104 may determine whether the patient's heart rate is above a recommended heart rate threshold, or at least a certain percentage more than the recommended heart rate threshold, for the age and sex of the patient 100. For instance, the remote server 104 may determine whether the patient's resting heart rate is above the resting heart rate threshold shown in Table 5 for the patient's age. As another example, the remote server 104 may determine whether the patient's resting heart rate is at least a predetermined percentage (e.g., 5%, 10%, 15%, 20%, 25%, etc.) above the resting heart rate threshold shown in Table 5 for the patient's age. If the patient's resting heart rate is above the resting heart rate threshold, or the predetermined percentage above the threshold, the remote server 104 may determine that the patient's resting heart rate is indicative of potential heart failure from cardiotoxicity.

TABLE 5
Resting Heart Rate Thresholds Associated
with Heart Failure According to Age

| | Resting Heart Rate |
|---|---|
| Age | Threshold (bpm) |
| 40 | 153 |
| 45 | 149 |
| 50 | 145 |
| 55 | 140 |
| 60 | 136 |
| 65 | 132 |
| 70 | 128 |

In implementations, as discussed above, the biosignal-based data includes (or is processable to produce) cardiovibrational data for the patient 100. The remote server 104 may then analyze the patient's cardiovibrational data to identify at least one cardiovibrational biomarker as part of process 600 of FIG. 9. For example, in healthy hearts, a cardiovibrational signal may include at least two normal heart vibrations, often described as a “lub” and a “dub” (or “dup”). The heart vibrations occur in sequence with each heartbeat in a cardiac cycle. To illustrate, a cardiovibrational signal includes an S1 cardiovibrational biomarker (herein referred to interchangeably as “S1”) and an S2 cardiovibrational biomarker (herein referred to interchangeably as “S2”). The S1 cardiovibrational biomarker is produced by the closing of the AV valves, and the S2 cardiovibrational biomarker is produced by the closing of the semilunar valves (SL valves). More specifically, the S1 cardiovibrational biomarker represents the closing of the AV valves including the tricuspid valve positioned between the right atria and right ventricle and the mitral valve located between the left atria and left ventricle. The S2 cardiovibrational biomarker represents the closing of the SL valves, including the pulmonic valve that ejects blood to the lungs to get oxygen and is positioned between the right ventricle and the pulmonary artery, and the aortic valve that ejects oxygenated blood to the body and is positioned between the left ventricle and the aorta.

In addition to S1 and S2 cardiovibrational biomarkers, a cardiovibrational signal may include S3 and S4 cardiovibrational biomarkers (herein referred to interchangeably as “S3” and “S4” respectively). The S3 cardiovibrational biomarker is a cardiovibration that occurs soon after the normal two “lub-dub” cardiovibrational biomarkers (S1 and S2). The S3 cardiovibrational biomarker typically occurs at the beginning of the middle third of diastole during passive filling of the ventricles. The S3 cardiovibrational biomarker can be associated with heart failure, for example as a result of the vibration of blood hitting the walls of the ventricles which may be stiffer in a patient suffering from cardiac problems. For instance, in monitoring systems described here, the S3 cardiovibrational biomarker can be monitored to detect possible cardiac problems, such as a failing left ventricle as in dilated congestive heart failure (CHF). The S4 cardiovibrational biomarker is a cardiovibration that occurs before the S1 cardiovibrational biomarker as a result of the atria contracting to push blood into the ventricles. The presence of an S4 cardiovibrational biomarker in a patient's cardiovibrational signal may be indicative of heart failure, as it is often caused by increased resistance to the filling of the ventricles from a reduction in ventricular wall compliance. The S4 cardiovibrational biomarker may also be associated with higher ventricular end-diastolic pressure.

As such, the remote server 104 may apply a feature extractor to a cardiovibrational signal of the patient 100 to identify S1, S2, S3, and/or S4 cardiovibrational biomarkers for the patient 100 (e.g., as part of performing step 608 of FIG. 9). The identified S1, S2, S3, and/or S4 cardiovibrational biomarkers may serve as cardiotoxicity biomarkers for the patient 100. The remote server 104 may then use the S1, S2, S3, and/or S4 cardiovibrational biomarkers to determine the current status of cardiotoxicity in the patient 100 (e.g., as part of performing step 610 of FIG. 9). For example, the remote server 104 may determine that the presence of S3 and/or S4 cardiovibrational biomarkers in the patient's cardiovibrational signal indicates an increased likelihood of heart failure. As another example, the remote server 104 may determine that the presence of S3 and/or S4 cardiovibrational biomarkers at or above a predetermined amplitude indicates an increased likelihood of heart failure.

As another example, the remote server 104 may determine an amplitude for the S1 and/or S2 cardiovibrational biomarkers. For instance, the remote server 104 may determine a normalized ratio of S1 and/or S2 amplitude relative to a baseline, outputting the normalized ratio as a number on a predetermined scale (e.g., between 0 and 1). The remote server 104 may then determine whether the S1 and/or S2 amplitude is above a predetermined threshold, as stronger S1 and/or S2 cardiovibrational biomarker may be associated with stronger heart valve closings and thus healthier contractions in the heart. For instance, the predetermined threshold may be a percentage of the patient's baseline S1 and/or S2 cardiovibrational biomarker amplitude, such as 80%, 75%, 70%, etc. of the patient's baseline S1 and/or S2 amplitude. Such lower-than-baseline S1 and/or S2 cardiovibrational biomarker may be indicative, for example, of pericardial effusion, with the buildup of fluid dampening the amplitude that can be sensed by the cardiovibrational sensor. As another example, the predetermined threshold may be a percentage over the patient's baseline S1 and/or S2 cardiovibrational biomarker amplitude, such as 120%, 124%, 130%, etc. of the patient's baseline S1 and/or S2 amplitude. The remote server 104 may determine the patient's baseline S1 and/or S2 cardiovibrational biomarker from signals gathered from the patient 100 while the patient 100 was using the wearable cardiotoxicity monitoring device 102 during a baselining session. If the S1 and/or S2 amplitude is above the predetermined threshold, the remote server 104 may determine that the patient 100 potentially has suffered cardiotoxicity due to their treatment regimen. For example, the increased S1 and/or S2 amplitude may be indicative of stiffer valve closures associated with heart failure.

As another example, the remote server 104 may determine a duration of the patient's S1 and/or S2 cardiovibrational biomarkers. Shorter S1 and/or S2 durations may also be associated with stronger heart valve closings. The remote server 104 therefore may determine whether the duration(s) of the patient's S1 and/or S2 biomarker are less than a predetermined threshold. To illustrate, the predetermined threshold may be percentage of the patient's baseline S1 and/or S2 cardiovibrational biomarker duration. For instance, the predetermined threshold may be 120%, 125%, 130%, etc. of the patient's baseline S1 and/or S2 cardiovibrational biomarker duration as determined from signals gathered from the patient 100 while the patient was using the wearable cardiotoxicity monitoring device 102 during a baselining session. If the S1 and/or S2 duration is not below the predetermined threshold, the remote server 104 may determine that the patient 100 potentially has suffered cardiotoxicity due to their treatment regimen.

In implementations, the biosignal-based data may include (or is processable to produce) cardiovibrational data and an ECG for the patient 100. The remote server 104 may then use the cardiovibrational data and the ECG to determine an electromechanical cardiac metric for the patient 100, where the at least one cardiotoxicity biosignal marker includes the electromechanical cardiac metric. The electromechanical cardiac metric may be determined as a timing between a predetermined ECG marker of the ECG signal for the patient 100 and a predetermined cardiovibrational biomarker of the cardiovibrational data for the patient 100. As an illustration, in examples, the remote server 104 may apply a feature extractor to identify parts of a cardiac cycle in the patient's ECG, such as P, Q, R, S, and T waves (e.g., using techniques discussed above with reference to processes 700, 710, 720, 740, and 760 of FIGS. 10-14). The remote server 104 may also apply a feature extractor to identify cardiovibrational biomarkers in the cardiovibrational signal for the patient 100 (e.g., using techniques discussed above). The remote server 104 may then determine a time interval between a fiducial point in the ECG signal and a fiducial point in the cardiovibrational signal.

For example, the remote server 104 may determine the electromechanical cardiac metric as the time difference between a P wave, a Q wave, an R wave, an S wave, or a T wave in the patient's ECG signal and an S1, S2, S3, or S4 cardiovibrational biomarker in a corresponding cardiac cycle of the patient's cardiovibrational data. As a specific illustration, the remote server 104 may determine the EMAT for the patient 100 as the time difference between a Q wave and an S1 cardiovibrational biomarker in a corresponding cardiac cycle. The remote server 104 may then use the electromechanical cardiac metric to determine the current status of cardiotoxicity in the patient 100 (e.g., as part of performing step 610 of FIG. 9). For instance, the remote server 104 may determine a baseline electromechanical cardiac metric for the patient 100 using signals generated and transmitted by the wearable cardiotoxicity monitoring device 102 during a baselining session for the patient 100. The remote server 104 may thus compare the current electromechanical cardiac metric for the patient 100 to the baseline electromechanical cardiac metric to determine whether the current electromechanical cardiac metric deviates from the baseline. To illustrate, the remote server 104 may determine that the current electromechanical cardiac metric shows that the patient 100 is likely experiencing cardiac issues when the current electromechanical cardiac metric is 10% greater, 15% greater, 20% greater, 25% greater, etc. than the baseline electromechanical cardiac metric for the patient 100.

In implementations, the remote server 104 may similarly analyze and apply bioacoustic data to the processes described above with respect to cardiovibrational data. For instance, the remote server 104 may receive bioacoustic data including information about the patient's valve movements, collected by a microphone, instead of cardiovibrational data including information about the patient's valve movements, collected by a cardiovibrational sensor (e.g., an accelerometer, such as the accelerometer 302 or a motion sensor 528). The remote server 104 may use the bioacoustics data instead of cardiovibrational data to identify the S1, S2, S3, and S4 biomarkers, as discussed above. In implementations, the remote server 104 may also use the bioacoustics data to identify one or more electromechanical cardiac metrics, as discussed above.

In implementations, as discussed above, the biosignal-based data includes (or is processable to produce) respiration data for the patient 100. For example, the wearable cardiotoxicity monitoring device 102 may gather accelerometer signals (e.g., from the accelerometer 302 or the at least one motion sensor 528) that include respiration data for the patient 100. As discussed above, the wearable cardiotoxicity monitoring device 102 may filter out lower frequency recordings corresponding to respiration movements of the patient 100 (e.g., using a low-pass filter) to produce respiration data for the patient 100. The wearable cardiotoxicity monitoring device 102 may then transmit the respiration data for the patient 100 (e.g., as part of step 604 of FIG. 9). Alternatively, in examples, the wearable cardiotoxicity monitoring device 102 may transmit the accelerometer signals to the remote server 104, which filters out the lower frequency recordings to produce respiration data for the patient 100 (e.g., as part of step 608 of FIG. 9). As another example, the wearable cardiotoxicity monitoring device 102 may include a transthoracic impedance sensor configured to sense respiration movements of the patient 100, and the respiration data may be transthoracic impedance data. The remote server 104 may use the respiration data to determine at least one cardiotoxicity biosignal marker for the patient 100 as part of applying step 610 of FIG. 9.

For example, the remote server 104 may divide the respiration data (e.g., filtered from the general accelerometer data) into recording intervals. Each recording interval may be checked for regularity characteristic of breathing cycles. This regularity may be marked, for instance, by a combination of requirements regarding signal peak and dip levels and distribution. For example, each recording interval may be compared to one or more breathing cycle templates to identify breathing cycles, and a certain number of breathing cycles may be compared to one another to determine if there is regularity in their signal peaks and dip levels. Once a “regular” stretch is configured, inhalation peaks are detected and counted. Respiration rate is then calculated as the average time interval between consecutive peaks in the regular stretch.

Systems and techniques for determining respiration from triaxial accelerometer data are described, for example, in “Respiratory rate and flow waveform estimation from tri-axial accelerometer data” by A. Bates et al. and published in 16 Sensors 750 (2016). As described by Bates et al., the accelerometer is assumed worn on the chest of the patient. In this case, if the patient is still, then the measured and normalized acceleration vector, a, will be close to the acceleration due to gravity, g, because the linear accelerations due to breathing would be relatively small. As noted in Bates et al., “as the accelerometer rotates, the gravity vector will rotate in the co-ordinate frame of the device. The axis of this rotation may be arbitrarily oriented in the device frame, and may change due to differences in the way the patient breathes at different times. It can also change with the orientation of the patient.” More specifically, the angle θₜ and the axis of rotation rₜ between consecutive measurements of the acceleration vector at times t−1 and t were determined by dot and cross products as follows:

$${θ}_{t} = {cos}^{- 1} ( {a}_{t} \cdot {a}_{t - 1} )$$

$${r}_{t} = {a}_{t} \times {a}_{t - 1}$$

Because oscillatory rotation rₜ, inverts when the direction of rotation reverses, Bates et al. normalized the axis direction by comparison to a reference axis r_{ref} as follows:

$${r}_{t}^{′} \{ \begin{matrix}{r}_{t} , & {r}_{t} \cdot {r}_{ref} \geq 0 \\ - {r}_{t} , & {r}_{t} \cdot {r}_{ref} < 0\end{matrix}$$

The predominant axis of rotation was then identified from measurements over a window of length W. Additionally, to reduce the noise, the estimate was weighted by the angle θₜ associated with each measurement, and to weight measurements closer in time to t, a Hamming window function H(n) was used. This process may be represented as follows:

$${\overline{r}}_{t} = normalize ( {\sum }_{i = - W / 2}^{W / 2} H ( i ) {θ}_{t + i} {r}_{t + i}^{′} )$$

The central point of the present rotations was estimated similarly:

$${\overline{a}}_{t} = normalize ( {\sum }_{i = - W / 2}^{W / 2} {a}_{t + i} )$$

Using these values, the current rotation angle ϕₜ, measured from the mean direction of gravity, was determined as follows:

$${ϕ}_{t} = {sin}^{- 1} ( ( {\overline{a}}_{t} \times {\overline{r}}_{t} ) \cdot {a}_{t} )$$

The angular velocity on the axis ωₜ was then determined as the derivative of ϕₜ:

$${ω}_{t} \frac{d ϕ}{dt}$$

The angular rate in radians per second is used to determine the respiration rate. For example, thresholds in the angular rate signals, scaled based on the root mean square (RMS) level of the signal, and dividing the amplitude range of the signal into low, middle and high bands can be performed. Accordingly, identification of a complete breath can be established using a state machine, where the signal transitions through a middle, a high, the middle, a low and then middle bands, in order to register a breath. The instantaneous respiratory rate is then given by the number of such qualifying breaths within a predetermined period of time.

In embodiments, the remote server 104 classifies the patient's respiration over time into various respiration types. The respiration types may include, for example, “regular breathing,” “rapid breathing,” “shallow breathing,” “rapid shallow breathing,” “deep breathing,” “wheezing,” “sleep disordered breathing,” “Cheyne-Stokes respiration,” and/or the like. As an illustration, the remote server 104 may either determine or receive from the motion sensor (e.g., the accelerometer 302 or the at least one motion sensor 528) a signal indicating the respiration rate of the patient 100 over time. The remote server 104 may compare the respiration rates to various respiration thresholds to classify whether a patient's respiration rate at any time fits under various categories, such as “regular breathing,” “shallow breathing,” and “deep breathing.” For instance, the remote server 104 may determine that the patient 100 was experiencing regular breathing when the patient 100 was breathing between 12 and 20 breaths per minute, that the patient 100 was experiencing shallow breathing when the patient 100 was breathing over 20 breaths per minute, and that the patient 100 was experiencing deep breathing when the patient 100 was breathing less than 12 breaths per minute. In some cases, the respiration thresholds used to determine which category the patient's respiration rate fits into are determined on a patient-by-patient basis, such as based on a respiration rate baseline for the patient 100 taken by a clinician.

The remote server 104 may then use the patient's respiration rate to determine the current status of cardiotoxicity in the patient 100 (e.g., as part of performing step 610 of FIG. 9). For example, the remote server 104 may use the patient's respiration rate to classify the patient's breathing as described above. If the patient's breathing absent patient activity (e.g., as determined from accelerometer counts recorded by the accelerometer 302 or the at least one motion sensor 528) is irregular a predetermined percentage of the time, the remote server 104 may determine that the patient 100 is expressing potential signs of heart failure from cardiotoxicity. To illustrate, the remote server 104 may determine that the patient 100 is experiencing potential cardiotoxicity if the patient shows irregular breathing 20% of the time, 25% of the time, 30% of the time, 35% of the time, 40% of the time, 45% of the time, 50% of the time, and/or the like.

In embodiments, the remote server 104 may use the patient's respiration data to determine where the patient 100 falls on a respiration or respiration-related index. For example, the remote server 104 may use the patient's respiration data to classify the patient 100 under the rapid shallow breathing index (RSBI). As another example, the remote server 104 may use the patient's respiration data, along with a determination of when the patient 100 is asleep (e.g., based on accelerometer data showing that the patient 100 is at rest and lying down and respiration data showing regular breathing) to classify the patient 100 under the apnea-hypopnea index (AHI). As another example, the remote server 104 may use the patient's respiration data and a determination of when the patient 100 is asleep to classify how much time the patient 100 spends in rapid eye movement (REM) sleep. The remote server 104 may further use the patient's classification to determine a cardiotoxicity status for the patient 100. For instance, if the remote server 104 identifies that the patient's classification on the RSBI has worsened over time, the remote server 104 may determine that the patient 100 is likely suffering from toxicity to their cardiovascular systems from the chemotherapy and/or radiation therapy treatments.

In implementations, as discussed above, the biosignal-based data includes (or is processable to produce) RF-based measurements associated with a thoracic fluid level in the patient 100. To illustrate, when taking an RF measurement, the wearable cardiotoxicity monitoring device 102 including an RF sensor (e.g., implemented through at least one RF antenna 304a, 304b and the RF circuit) may transmit bursts of RF waves in a span of frequencies (e.g., a burst of waves in the 0.1 to 5.0 GHz range in at least 50 frequency steps). Thus, the RF measurement matrix may include rows, where each row represents a single RF stepped frequency measurement, and columns, where each column represents a single frequency step. For example, an RF measurement matrix may include 1000-5000 rows (e.g., 50 per second), with each row representing the burst number, and 50-100 columns, with each column representing the frequency number (e.g., from 1 to 50-100). In some implementations, the wearable cardiotoxicity monitoring device 102 may also generate a calibration vector. For instance, the wearable cardiotoxicity monitoring device 102 may take a calibration measurement prior to each RF measurement such that each element in the calibration vector refers to one of the transmitted RF frequencies. The calibration vector may represent the internal signal path and may be used to compensate for an internal sensor frequency response. The wearable cardiotoxicity monitoring device 102 may transmit the RF measurement matrix and calibration vector to the remote server 104 (e.g., as part of step 604 of FIG. 9).

The remote server 104 may process the received RF measurement matrix and calibration vector to determine a thoracic fluid level in the patient 100 as at least one cardiotoxicity biosignal marker (e.g., as part of step 608 of FIG. 9). In some cases, the remote server 104 may process the RF measurement matrix and calibration vector to determine a relative thoracic fluid level in the patient 100. For example, the wearable cardiotoxicity monitoring device 102 may take a baseline RF measurements when the patient 100 starts using the wearable cardiotoxicity monitoring device 102. The remote server 104 may thus determine whether the patient's current RF measurements are above or below their baseline and by what percentage. To illustrate, such relative thoracic fluid levels may be output as a thoracic fluid index or content level (THC). In other cases, the remote server 104 may process the RF measurement matrix and calibration vector to determine an absolute or actual thoracic fluid level. As such, the remote server 104 may calibrate the RF measurement matrix using the calibration vector, and use the filtered RF measurement matrix or a representative RF measurement value as one or more inputs to a thoracic fluid level equation. The thoracic fluid level equation is configured to output an actual thoracic fluid level of the patient 100, for example, based on predetermined clinical data from a cohort of patients. In examples, the remote server 104 may also use other patient measurements or demographic information as inputs to the thoracic fluid level equation, such as the patient's BMI, heart rate, age, and/or the like. The remote server 104 may then use the patient's thoracic fluid level to determine the current status of cardiotoxicity in the patient 100 (e.g., as part of performing step 610 of FIG. 9).

For example, if the patient's thoracic fluid level is a relative thoracic fluid level, the remote server 104 may determine if the relative level has transgressed a predetermined threshold indicative of a certain worsening from the patient's baseline (e.g., 10%, 15%, 20%, 25%, 30%, 35%, 40%, and/or the like). As another example, if the patient's thoracic fluid level is an absolute thoracic fluid level, the remote server 104 may determine if the absolute thoracic fluid level has transgressed a predetermined fluid level threshold (e.g., greater than 30 mL, greater than 40 mL, greater than 50 mL, etc.) or predetermined volume percentage (e.g., greater than 10%, greater than 15%, greater than 20%, etc.). As another example, if the patient's thoracic fluid level is an absolute thoracic fluid level, the remote server 104 may determine if the patient's thoracic fluid level transgresses a predetermined percentage from a baseline thoracic fluid level (e.g., determined from signals gathered and transmitted by the wearable cardiotoxicity monitoring device 102 during a baselining period), similar to the relative level discussed above.

In implementations, as discussed above, the biosignal-based data includes (or is processable to produce) blood pressure data for the patient 100. As an illustration, the wearable cardiotoxicity monitoring device 102 may generate RF data including information about the patient's aortic waveform (e.g., the waveform of the aorta's volumetric cross-section over time) and PPG data including information about the patient's surface arterial waveform (e.g., the waveform of volume of the patient's surface arteries over time). The wearable cardiotoxicity monitoring device 102 may then determine the patient's blood pressure from fiducial points on the patient's aortic and surface arterial waveforms as described above. Alternatively, or additionally, the wearable cardiotoxicity monitoring device 102 may transmit the RF data and the PPG data to the remote server 104, and the remote server may determine the patient's blood pressure from fiducial points on the patient's aortic and surface arterial waveforms as described above (e.g., as part of steps 608 of FIG. 9). The patient's blood pressure may accordingly be at least one cardiotoxicity biomarker for the patient 100. In implementations, the remote server 104 may determine the patient's systolic blood pressure. In implementations, the remote server 104 may determine the patient's systolic blood pressure and diastolic blood pressure.

The remote server 104 may then use the patient's blood pressure to determine the current status of cardiotoxicity in the patient 100 (e.g., as part of performing step 610 of FIG. 9). For instance, the remote server 104 may compare the patient's blood pressure to the thresholds shown in Table 5 below. If either of the patient's blood pressures fall into the medium or high risk categories, the remote server 104 may determine that the patient 100 may have suffered some cardiotoxicity and/or that the patient 100 needs further monitoring. As an example, if the patient 100 had no risk from their blood pressure before starting an oncology treatment regimen, but enters a medium or high risk category during the oncology treatment, the remote server 104 may determine that the patient 100 has potentially suffered some cardiotoxicity. As another example, if the patient 100 was in the medium or high risk category before starting an oncology treatment regimen, the remote server 104 may determine whether the patient's blood pressure risk level has worsened. If so, the remote server 104 may determine that the patient 100 has potentially suffered some cardiotoxicity.

TABLE 6
Risk Levels According to Blood Pressure

| Systolic | Diastolic | Risk |
|---|---|---|
| Blood Pressure | Blood Pressure | Level |
| Less than | Less than | No risk |
| 120 mm Hg | 80 mm Hg | |
| 120-139 mm Hg | 80-89 mm Hg | Medium risk |
| | | (prehypertension) |
| 140 mm Hg | 90 mm Hg | High risk |
| or higher | or higher | (hypertension) |

In implementations, as discussed above, the remote server 104 may receive additional sensor data from the wearable cardiotoxicity monitoring device 102. For example, the wearable cardiotoxicity monitoring device 102 may record one or more non-biosignals. The non-biosignals may include signals from additionally applied external sensors configured to sense non-biosignals and/or signals that include additional non-biosignal information. As an illustration, the remote server 104 may receive an accelerometer signal from the wearable cardiotoxicity monitoring device 102 including biosignal information or data (e.g., low frequency accelerometer counts corresponding to respiration) and non-biosignal information or data (e.g., high frequency accelerometer counts corresponding to patient movement and posture information recorded based on the effect of gravity on the sensor). In such implementations, in addition to analyzing the biosignal-based data from the wearable cardiotoxicity monitoring device 102, the remote server 104 may analyze the additional sensor data to identify at least one additional cardiotoxicity marker associated with heart failure caused by chemotherapy cardiotoxicity and/or radiation therapy cardiotoxicity (e.g., as part of applying step 608 of FIG. 9). The remote server 104 may also determine the current status of cardiotoxicity in the patient 100 further based on the at least one additional cardiotoxicity marker (e.g., as part of applying step 610 of FIG. 9).

For example, the remote server 104 may determine body position or posture from a motion signal received from the wearable cardiotoxicity monitoring device 102. The motion sensor (e.g., the accelerometer 302 of the cardiotoxicity monitoring unit 106 or the at least one motion sensor 528 of the garment-based monitoring device 118) may include a 3D accelerometer with three axes, and the motion signals may capture to posture information for the patient 100. More specifically, the posture may be calculated by measuring the projected earth gravity vector in each of three axes of the accelerometer. The device tilt may thus be measured as the arcsine of the ratio between the measured acceleration and earth's gravitational factor (g). In some cases, this body posture determination may be made at the remote server 104, which receives, for example, the projected earth gravity vectors from the motion sensor of wearable cardiotoxicity monitoring device 102. In other cases, this determination may be made by the wearable cardiotoxicity monitoring device 102, which transmits motion signals that include a device tilt signal that corresponds to the body position or posture for the patient 100 over time to the remote server 104.

As an illustration, in implementations, the wearable cardiotoxicity monitoring device 102 may include a tri-axial accelerometer sensitive to the gravitational field, where the accelerometer reading samples during the measurement time window may be represented as x₁, y₁, and z₁. For each axis, the remote server 104 may normalize the accelerometer samples by the g (gravity) value. The remote server 104 may then average the accelerometer measurements over the measurement time window, which may be represented as:

$${x}_{a} = mean ( {x}_{i} )$$

$${y}_{a} = mean ( {y}_{i} )$$

$${z}_{a} = mean ( {z}_{i} )$$

If the motion sensor is being worn on the patient's front (e.g., on the patient's chest), the remote server 104 may apply an angle transformation to the averaged accelerometer measurements. This transformation rotates the axis reference from the front location to a side location to bring the data to a common frame of reference (e.g., a common frame of reference with accelerometer data from accelerometers worn on the patient's side). To do this transformation, the remote server 104 defines rotation matrices ROT₁ and ROT₂ as follows:

$$R O {T}_{1} = [ \begin{matrix}cos ( θ ) & - sin ( θ ) & 0 \\ sin ( θ ) & cos ( θ ) & 0 \\ 0 & 0 & 1\end{matrix} ]$$

$$RO {T}_{2} = [ \begin{matrix}1 & 0 & 0 \\ 0 & cos ( α ) & - sin ( α ) \\ 0 & sin ( α ) & cos ( α )\end{matrix} ]$$

where α=24° and 0=45°. The remote server 104 then rotates the acceleration vector as follows:

$$[ \begin{matrix}{x}_{r} \\ {y}_{r} \\ {z}_{r}\end{matrix} ] = [ \begin{matrix}{x}_{a} \\ {y}_{a} \\ {z}_{a}\end{matrix} ] * {ROT}_{1} * {ROT}_{2}$$

For accelerometer data normalized to a common frame of reference, the remote server 104 may then calculate pitch and roll using the formulas:

$$pitch = a tan 2 ( - {y}_{r} , \sqrt{{x}_{r}^{2} + {z}_{r}^{2}} )$$

$$roll = a tan 2 ( {z}_{r} , \sqrt{{y}_{r}^{2} + μ * {x}_{r}^{2}} )$$

where atan2 is the four quadrant inverse tangent, μ=0.1, and μ*xᵣ² is used to remove calculation instabilities. Additionally, in the formula above, the pitch angle is the tilt angle from a vertical orientation. This angle is used to determine if the patient 100 is supine, reclined, or upright.

In some embodiments, the remote server 104 classifies the patient's posture over time into various posture types. The posture types may include, for example, “supine,” “reclined,” “upright,” “standing,” “sitting,” “resting,” “lying on the right side,” “lying on the left side,” and/or the like. To illustrate, the remote server 104 may either determine or receive from the motion sensor a signal indicating the device tilt of the motion sensor of the wearable cardiotoxicity monitoring device 102 in degrees over time. The remote server 104 may compare the degree of the motion sensor's tilt to various degree thresholds to classify whether a patient's posture at any given time fits under various categories, such as “supine,” “reclined,” and “upright.” For instance, the remote server 104 may determine that the patient 100 was supine where the motion sensor's tilt (e.g., the patient's pitch angle) was less than 30 degrees, determine that the patient 100 was reclined when the motion sensor's tilt was between 30 degrees and 60 degrees, and determine that the patient 100 was upright when the motion sensor's tilt was greater than 60 degrees.

The posture for the patient 100 may also be calibrated, for example, the first time that the patient wears the wearable cardiotoxicity monitoring device 102 to determine an upright posture for the patient 100. As an example, the patient 100 may wear the wearable cardiotoxicity monitoring device 102 in a caregiver's office during a baselining session. The caregiver may indicate to the wearable cardiotoxicity monitoring device 102 (e.g., via a user interface), or directly to the remote server 104 via a caregiver interface 116, when the patient 100 is upright versus reclined. The remote server 104 may then set degree thresholds for classifying posture based on this calibration.

As another example, the remote server 104 may determine an activity level for the patient 100 using a motion signal received from the wearable cardiotoxicity monitoring device 102. The motion sensor may include an accelerometer (e.g., the accelerometer 302 or the at least one motion sensor 528 implemented as including an accelerometer), and the motion signals may capture activity information for the patient 100. In particular, activity may be measured in accelerometer counts, which are correlated with energy expenditure due to physical activity (e.g., a commonly accepted measure of activity level). Counts may be calculated by taking patient acceleration, removing the component exerted by the Earth's gravity, taking the absolute value, and integrating over time. In implementations, determining the activity information for the patient 100 may include determining accelerometer counts for a certain amount of time. For instance, the number of accelerometer counts per second may be determined. As another example, the number of accelerometer counts per second may be determined and multiplied by 60 to find the number of accelerometer counts per minute at any given time for the patient 100. As another example, these number of accelerometer counts per minute may be averaged over the course of a certain period of time, such as five minutes, to determine an average number of accelerometer counts over the period. In some cases, the count determination may be made at the remote server 104, which may receive raw or filtered accelerometer signals from the wearable cardiotoxicity monitoring device 102. In other cases, this determination may be made by the wearable cardiotoxicity monitoring device 102, which transmits motion signals that include the accelerometer counts, corresponding to activity information for the patient 100 over time, to the remote server 104.

In some embodiments, the remote server 104 classifies the patient's activity level over time into various activity level types. The activity level types may include, for example, “active,” “non-active,” “walking,” “low activity,” “intermediate activity,” “sleeping,” “high activity,” “patient fall,” and/or the like. As an illustration, the remote server 104 may either determine or receive from the motion sensor a signal indicating the accelerometer counts of the motion sensor over time. The remote server 104 may compare the accelerometer counts to various count thresholds and/or amplitude thresholds to classify whether a patient's activity level at any given time fits under various categories, such as “active” and “non-active.” For instance, the remote server 104 may determine that the patient 100 was active when the accelerometer counts per minute were 1000 or more and that the patient 100 was non-active when the accelerometer counts per minute were less than 1000. To illustrate another example, the remote server 104 may determine that the patient 100 was active when the accelerometer counts per second were greater than 15 for at least 30 seconds of a minute time interval and that the patient 100 was non-active the rest of the time.

As another example, the remote server 104 may determine that the patient 100 was in an “active” state for a given minute when the patient 100 showed activity (e.g., accelerometer counts above a predetermined threshold) for at least 30 seconds and had an average posture greater than 35 degrees. Additionally, the remote server 104 may determine that the patient 100 was in an “asleep” state for a given minute when the patient 100 showed activity for less than 12 seconds and had an average posture less than or equal to 35 degrees for that minute. The remote server 104 may finally determine that the patient 100 was in an “inactive” state if the patient 100 had a combination of activity and posture values for a given minute that did not satisfy the active or sleep state thresholds.

Once the remote server 104 has determined that patient's activity level and/or body position, the remote server 104 may then use the patient's activity level and/or body position to determine the current status of cardiotoxicity in the patient 100 (e.g., as a further part of performing step 610 of FIG. 9). As an example, the remote server 104 may determine when the patient 100 is asleep, as described above. The remote server 104 may then identify the patient's average body position while the patient 100 was sleeping. If the patient's average body position is above a predetermined threshold (e.g., above 30 degrees, above 45 degrees, etc.) and/or if the patient's average body position has increased over time while the patient 100 is sleeping, the remote server 104 may determine that the patient 100 is potentially experiencing an increase in thoracic fluid levels from cardiotoxicity. For instance, the amount of fluid in the patient's lungs may be making it difficult for the patient 100 to breathe while lying on their back such that the patient 100 is using pillow or other sleep aids to prop up their back while they're sleeping. As another example, the remote server 104 may determine that if the patient's average activity level has decreased over time, the patient 100 may be at greater risk of having suffered cardiotoxicity that is making it difficult for the patient to engage in activity.

In implementations, the remote server 104 may determine the current status of cardiotoxicity in the patient 100 using a combination of different cardiotoxicity biosignal markers and/or additional cardiotoxicity markers. To illustrate, the remote server 104 may use a variety of cardiotoxicity biosignal markers as inputs to a cardiotoxicity evaluation function, as described above. As another illustration, the remote server 104 may determine an overall cardiac health for the patient 100 as part of determining the patient's current cardiotoxicity status. For example, the remote server 104 may evaluate five factors of cardiac health for the patient 100, including PR interval duration, QRS complex duration, QT interval duration, thoracic fluid level, and body position during sleep. If all five factors are within recommended levels, the remote server 104 may determine that the patient 100 is at low risk for having suffered cardiotoxicity. If at least two factors are above recommended levels, the remote server 104 may determine that the patient 100 is at medium risk for having suffered cardiotoxicity. If at least four factors are above recommended levels, the remote server 104 may determine that the patient 100 is at high risk for having suffered cardiotoxicity. Alternatively, or additionally, the remote server 104 may determine a cardiac health score for the patient 100 based on the evaluated cardiotoxicity markers, such as a score out of 100.

As another illustration, the remote server 104 may flag patient data based on a combination of cardiotoxicity biosignal markers. As an example, the remote server 104 may use the patient's posture, activity level, and respiration rate to determine when the patient 100 is sleeping. However, if the remote server 104 identifies a heart rate level much higher than the patient's normal heart rate while the patient 100 is sleeping (e.g., 20% above their baseline level, 25% above their baseline level, 30% above their baseline level, and/or the like), the remote server 104 may flag the patient's ECG data corresponding to the elevated heart rate for the patient's physician to review. As another example, the remote server 104 may identify when the patient's respiration rate is elevated at night and/or while the patient 100 is determined to be sleeping (e.g., 20% above their baseline respiration rate, 25% above their baseline respiration rate, 30% above their baseline respiration rate, and/or the like).

In implementations, the remote server 104 may determine one or more trends as part of determining the current cardiotoxicity status of the patient 100. In examples, the remote server 104 may determine at least one cardiotoxicity biosignal (or non-biosignal) marker on a periodic basis (e.g., hourly, daily, weekly, biweekly, monthly, etc.). The remote server 104 may store the patient's cardiotoxicity markers in a database, along with previously determined cardiotoxicity markers. The remote server 104 may then generate a trend for the determined cardiotoxicity markers, such as by plotting the cardiotoxicity markers over time. Based on the trend, the remote server 104 may determine the patient's current cardiotoxicity status. For example, if a cardiotoxicity biosignal (or non-biosignal) marker has deviated from a baseline amount recorded for the patient 100 by a predetermined amount or percentage (e.g., by 10%, 15%, 20%, etc.), the remote server 104 may determine that the patient 100 is at risk for having suffered cardiotoxicity due to the chemotherapy and/or radiation therapy. However, the remote server 104 may also determine that the patient 100 is at risk for having suffered cardiotoxicity due to chemotherapy and/or radiation therapy when the trendline is at or above a certain slope, such as at or above a 0.2 slope, 0.25 slope, 0.3 slope, 0.4 slope, 0.5 slope, 0.75 slope, 1.0 slope, etc. As another example, if a cardiotoxicity marker shows a trend over time in a certain direction (e.g., increasing PR interval durations, increasing QRS complex durations, increasing QT interval durations, increasing thoracic fluid, and/or the like), the remote server 104 may determine that the patient 100 is trending towards cardiotoxicity, even if the patient 100 currently does not show levels of cardiotoxicity that need to be immediately addressed.

Returning to FIG. 9, after determining the current cardiotoxicity status of the patient 100, the remote server 104 generates an output based on the current cardiotoxicity status at step 612. For example, the remote server 104 may interface with a caregiver interface 116 to display a dashboard, such as a physician dashboard (e.g., as described above). The physician dashboard display reports for various patients, including the patient 100, of the associated physician. To illustrate, the physician dashboard may include various metrics for various patients, including the patient 100, of the associated physician. The physician dashboard may also highlight patients that the remote server 104 has determined may have suffered heart failure due to chemotherapy and/or radiation therapy cardiotoxicity, examples of which have been described above. In implementations, the physician dashboard may also display trends for patients over time (e.g., based on periodic measurements for each patient). For instance, the physician dashboard may include tabs or buttons that allow the physician to view a given patient's PR interval durations over time, QRS complex durations over time, QT interval durations over time, heart rate over time, thoracic fluid level over time, body position during sleep over time, activity level over time, and/or the like. Alternatively, or additionally, the remote server 104 may transmit a report with various metrics for the patient 100 more directly to the patient's physician. For instance, the remote server 104 may email the patient's physician the report on the patient 100.

As another example, the remote server 104 may determine a cardiac risk assessment for the patient 100, such as an overall risk score for the patient 100 based on a number of cardiotoxicity biosignal markers. The remote server 104 may then display the risk score on the physician dashboard. In some cases, the risk score may also include breakdowns of independent components making up the score, such as indicators of heart block in the patient 100, the patient's heart fluid level, risk factors observed in the patient's T waves, and/or the like.

As another example, the remote server 104 may transmit an alert to the patient's caregiver and/or to the patient 100, as also described above. For example, the remote server 104 may transmit an alert to the patient's physician when at least one cardiotoxicity biosignal marker transgresses a predetermined threshold associated with imminent risk of heart failure. As another example, the remote server 104 may transmit an alert to the patient 100 when at least one cardiotoxicity biosignal marker transgresses a predetermined threshold associated with imminent risk of heart failure, warning the patient 100 that the patient 100 should see their prescribing physician regarding their oncology treatment plan as soon as possible.

As another example, an output of step 612 may be decision-making regarding monitoring and/or treating the patient 100. To illustrate, if the patient's current cardiotoxicity status is above a predetermined threshold, the remote server 104 may determine that the patient 100 should be more closely monitored (e.g., the remote server 104 should determine the patient's current cardiotoxicity status on a weekly basis instead of a biweekly basis). As another illustration, the remote server 104 may provide one or more recommendations to the patient's caregiver. For instance, the remote server 104 may recommend to the patient's prescribing physician that dosages of the patient's chemotherapy and/or radiation treatments should be decreased, that the patient's chemotherapy and/or radiation treatment should be suspended until the patient's cardiac health improves, that the patient 100 should see a cardiologist for heart failure treatments, and/or the like.

In implementations, the output at step 612 may include a correlation of patient symptoms recorded by the wearable cardiotoxicity monitoring device 102 with the biosignal-based data. This type of output may allow the patient's physician to understand whether recorded symptoms are related to cardiovascular effects of the chemotherapy and/or radiation therapy or whether the recorded symptoms are directly due to the chemotherapy and/or radiation therapy on target organs producing symptoms, such as nausea and lightheadedness. In implementations, the wearable cardiotoxicity monitoring device 102 may record an input indicating that the patient 100 is experiencing a symptom, record one or more biosignal segments and/or additional signals in association with the symptom input, and transmit the symptom input and recorded one or more biosignal segments to the remote server 104. For example, where the wearable cardiotoxicity monitoring device 102 includes a cardiotoxicity monitoring unit 106 and a portable gateway 110, the cardiotoxicity monitoring unit 106 and/or the portable gateway 110 may record the symptom input and the one or more biosignal segments and/or additional signal segments as described above. As another example, where the wearable cardiotoxicity monitoring device 102 includes the garment-based monitoring device 118, the medical device controller 406 of the garment-based monitoring device 118 and/or a portable gateway 110 in communication with the garment-based monitoring device 118 may similarly record a symptom input and one or more biosignal segments and/or additional signal segments in association with the symptom input. The wearable cardiotoxicity monitoring device 102 may then transmit the recorded symptom input and one or more biosignal segments to the remote server 104 as part of the biosignal-based data at step 604.

As part of the output at step 612, the remote server 104 may produce a chart or graph illustrating the patient's recorded symptoms along with the one or more biosignal segments and/or additional signal segments such that the patient's physician may view activity in the patient's biosignals at the time of the recorded symptom input. The remote server 104 may, for example, transmit the chart or graph to the caregiver interface 116 associated with the patient's physician. As another example, the remote server 104 may display the chart or graph as part of an information portal that the patient's physician logs on to access. To illustrate, the remote server 104 may display the patient's recorded symptoms along with the patient's ECG, activity level, posture, and respiration rate within a predetermined time period, such as 1 minute, around the symptom input. In implementations, the remote server 104 may additionally or alternatively use the recorded one or more biosignals to determine other patient data to include in the chart or graph. For instance, the remote server 104 may use accelerometer signals and ECG signals to determine the patient's activity level, posture, respiration rate, and heart rate at the time of a symptom input. Using the patient's activity level, posture, respiration rate, and heart rate, the remote server 104 may determine whether the patient was sleeping at the time of the symptom input and display the patient's sleep status as part of the chart or graph.

In implementations, the remote server 104 may display prescription information alongside, for example, biosignal information for the patient 100. For example, the remote server 104 may receive prescription information from the patient's physician indicating when the patient 100 is beginning a new chemotherapy and/or radiation therapy treatment. The remote server 104 may then display a chart or graph of the patient's biosignal information (and, in some cases, non-biosignal information) over time in conjunction with the prescription information so that the patient's physician can better visualize physiological changes that have occurred in conjunction with the new treatment regime. As another example, the patient 100 may indicate to the remote server 104 when the patient 100 is engaging in a treatment (e.g., taking a chemotherapeutic drug or going in for a radiation treatment). The patient 100 may indicate that the patient 100 is engaging in a treatment using a similar process to recording a symptom, as described above. The remote server 104 may then display the patient's treatment times on a chart or graph of the patient's biosignal information (and, in some cases, non-biosignal information) for the patient's physician to review.

In some cases, it may be desirable to determine one or more baselines for the patient 100 before the patient 100 begins chemotherapy and/or radiation therapy. As an illustration, as described in examples above, the remote server 104 may use baseline data for the patient 100 to later identify the cardiotoxicity status in the patient 100. As another illustration, the remote server 104 may use baseline data for the patient 100 to determine whether the patient 100 is healthy enough to begin chemotherapy and/or radiation therapy. As another illustration, the remote server 104 may use baseline data for the patient 100 to recommend some or all of the patient's oncology treatment regime, such as a dosage for chemotherapy. As another illustration, the remote server 104 may additionally or alternatively transmit the baseline data to the patient's physician so that the physician may determine whether the patient 100 is healthy enough to begin treatment or use the baseline data to determine the patient's oncology treatment regime.

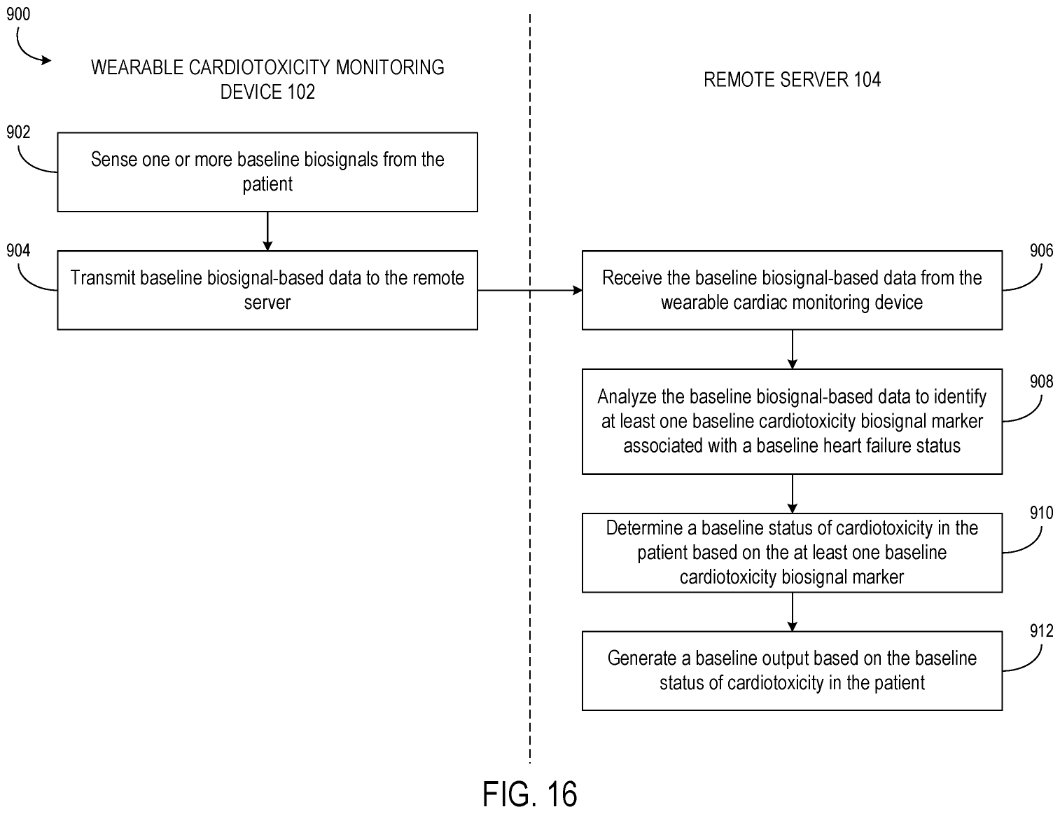

FIG. 16 illustrates a sample process flow for monitoring baseline cardiotoxicity biosignal markers in oncology patients. The sample process 900 shown in FIG. 16 can be implemented by the wearable cardiotoxicity monitoring device 102 being used by the patient 100 and the remote server 104. The wearable cardiotoxicity monitoring device 102 is configured to sense one or more baseline biosignals from the patient 100 at step 902. In implementations, the wearable cardiotoxicity monitoring device 102 may perform the process of step 902 similarly to the process of step 602, described above with reference to FIG. 9. In implementations, the wearable cardiotoxicity monitoring device 102 is configured to sense the one or more baseline biosignals during a baselining period or session performed before the patient 100 begins chemotherapy and/or radiation therapy. For example, the baselining period may be performed during a physician appointment, or the baselining period may be performed during a time period (e.g., a week, two weeks, a month, and/or the like) before the patient 100 begins oncology treatment. The wearable cardiotoxicity monitoring device 102 is configured to transmit baseline biosignal-based data based on the one or more sensed biosignals to the remote server 104 at step 904. In implementations, the wearable cardiotoxicity monitoring device 102 may perform the process of step 904 similarly to the process of step 604, described above with reference to FIG. 9.

The remote server 104 is configured to receive the baseline biosignal-based data from the wearable cardiotoxicity monitoring device 102 at step 906. In implementations, the remote server 104 is configured to perform the process of step 906 similarly to the process of step 606, described above with reference to FIG. 9. The remote server 104 is configured to analyze the baseline biosignal-based data from the wearable cardiotoxicity monitoring device 102 to identify at least one baseline cardiotoxicity biosignal marker associated with a baseline heart failure status (e.g., the patient's heart failure status before the patient 100 undergoes chemotherapy and/or radiation therapy) at step 908. In implementations, the remote server 104 is configured to perform the process of step 908 similarly to the process of step 608, described above with reference to FIG. 9. The remote server 104 is configured to determine a baseline status of cardiotoxicity in the patient 100 based on the at least one baseline cardiotoxicity biosignal marker at step 910. As an illustration, the remote server 104 may use similar processes as described above with respect to steps 608 and 610 to determine one or baselines for the patient 100 that the remote server 104 may use later during the patient's oncology treatments to identify the patient's current cardiotoxicity status.

In implementations, the remote server 104 is further configured to generate a baseline output based on the baseline status of cardiotoxicity in the patient 100 at step 912. As an example, the baseline output may include one or more baseline metrics for the patient 100 that the remote server 104 stores for the purpose of determining the patient's current cardiotoxicity status once the patient 100 begins chemotherapy and/or radiation therapy, as described in more detail with respect to steps 608 and 610 of FIG. 9. As another example, the baseline output may be similar to the output generated at step 612 of FIG. 9, such as information provided on a physician dashboard. As another example, the baseline output may include a cardiac risk assessment for the patient 100. To illustrate, the baseline output may determine the patient's current heart failure status and whether the patient 100 is healthy enough to begin the planned treatment regimen. If the remote server 104 determines that the patient 100 is not healthy enough to begin the planned treatment regimen, the remote server 104 may recommend one or more adjustments to the patient's proposed treatment regimen or an alternate treatment regimen with fewer risks to the patient's cardiac health.

In some cases, it may be desirable to continue monitoring the patient's cardiac health after the patient 100 has completed a treatment regimen. For instance, some cardiotoxicity effects may not be immediately detectable, and as such, further monitoring may catch cardiac side effects from the chemotherapy and/or radiation therapy that may require further treatment or observation. As another example, some cardiotoxicity effects may decrease over time once the patient's chemotherapy and/or radiation therapy has been completed, which may be beneficial for the patient 100 and their caregivers to be aware of for future health screenings. In implementations, the patient 100 may use the wearable cardiotoxicity monitoring device 102 for a period of time after their oncology treatment has finished, such as two weeks, four weeks, six weeks, eight weeks, and/or the like after completion of their oncology treatment. In implementations, the patient 100 may use the wearable cardiotoxicity monitoring device 102 periodically after their oncology treatment has finished. For example, the patient 100 may use the wearable cardiotoxicity monitoring device 102 for two weeks every six months after the completion of their oncology treatment, discontinuing the periodic use after two years if no serious lasting side effects from the oncology treatment have been detected.

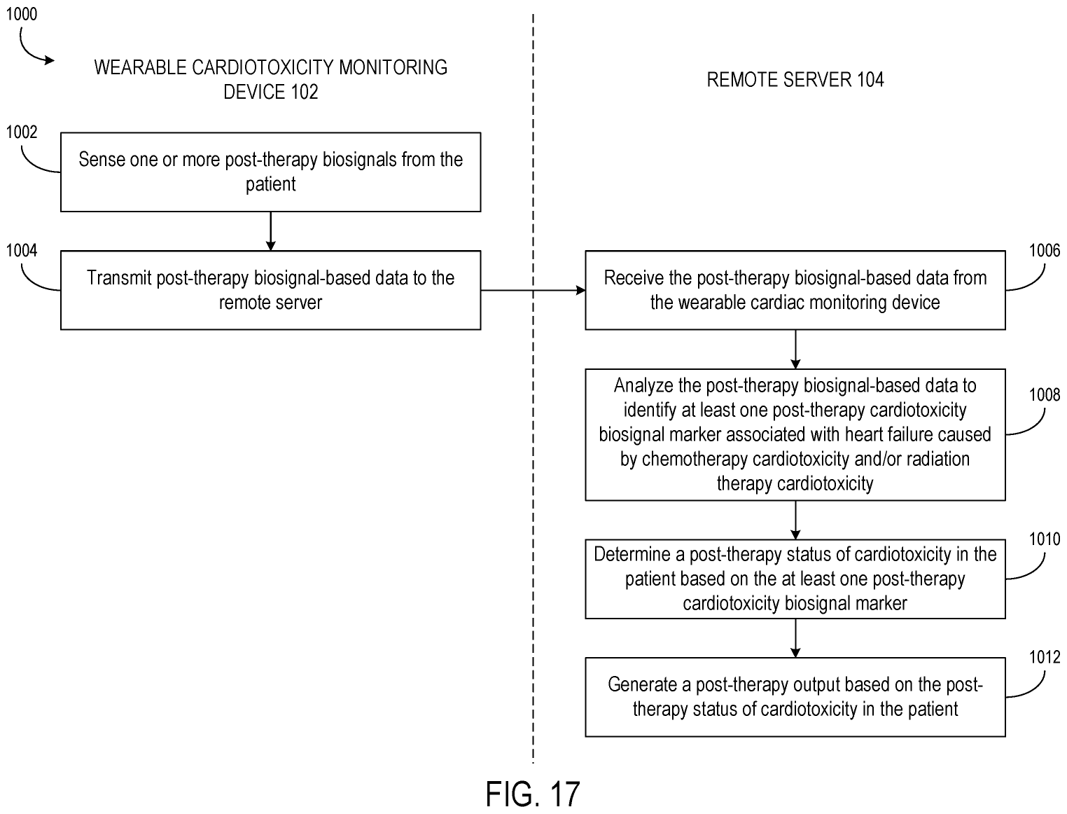

FIG. 17 illustrates a sample process flow for monitoring post-therapy cardiotoxicity biosignal markers in oncology patients. The sample process 1000 shown in FIG. 17 can be implemented by the wearable cardiotoxicity monitoring device 102 being used by the patient 100 and the remote server 104. The wearable cardiotoxicity monitoring device 102 is configured to sense one or more post-therapy biosignals from the patient 100 at step 1002. In implementations, the wearable cardiotoxicity monitoring device 102 is configured to perform the process of step 1002 similarly to the process of step 602, described above with reference to FIG. 9. The remote server 104 is configured to transmit the post-therapy biosignal-based data based on the one or more sensed biosignals to the remote server 104 at step 1004. In implementations, the wearable cardiotoxicity monitoring device 102 is configured to perform the process of step 1004 similarly to the process of step 604, described above with reference to FIG. 9.

The remote server 104 is configured to receive the post-therapy biosignal-based data from the wearable cardiotoxicity monitoring device 102 at step 1006. In implementations, the remote server 104 is configured to perform the process of step 1006 similarly to the process of 606, described above with reference to FIG. 9. The remote server 104 is configured to analyze the post-therapy biosignal-based data from the wearable cardiotoxicity monitoring device 102 to identify at least one post-therapy cardiotoxicity biosignal marker associated with heart failure caused by the patient's chemotherapy and/or radiation therapy regimen at step 1008. In implementations, the remote server 104 is configured to perform the process of step 1008 similarly to the process of step 608, described above with respect to FIG. 9. The remote server 104 is configured to determine a post-therapy status of cardiotoxicity in the patient 100 based on the at least one post-therapy cardiotoxicity biosignal marker at step 1010. In implementations, the remote server 104 is configured to perform the process of step 1010 similarly to the process of step 610, described above with respect to FIG. 9. For example, the remote server 104 may use the processes described above with respect to steps 608 and 610 in similar ways, except using this post-therapy data collected from the patient 100.

In implementations, the remote server 104 is further configured to generate a post-therapy output based on the post-therapy status of cardiotoxicity in the patient 100 at step 1012. The remote server 104 may perform the process of step 1012 similarly to the process of step 612, described above with reference to FIG. 9. As an illustration, the remote server 104 may similarly alert the patient 100 or the patient's caregiver, display the patient's post-therapy data on a physician dashboard or portal, output a cardiotoxicity rating for the patient 100, and/or the like. In examples, the remote server 104 may recommend one or more follow-up actions or monitoring for the patient 100. For instance, if the remote server 104 determines that the patient 100 has entered second- or third-degree heart block, the remote server 104 may recommend that the patient 100 receive an implantable pacemaker.

In implementations, any of the above analysis or actions described as being performed by the remote server 104 may instead be performed by the wearable cardiotoxicity monitoring device 102. For example, the wearable cardiotoxicity monitoring device 102 may determine at least one cardiotoxicity biosignal marker, determine a current cardiotoxicity status of the patient's heart, and transmit an output based on the current cardiotoxicity status to the remote server 104 (e.g., perform steps 608, 610, and 612 of FIG. 9; perform steps 908, 910, and 912 of FIG. 16; perform steps 1008, 1010, and 1012 of FIG. 17).

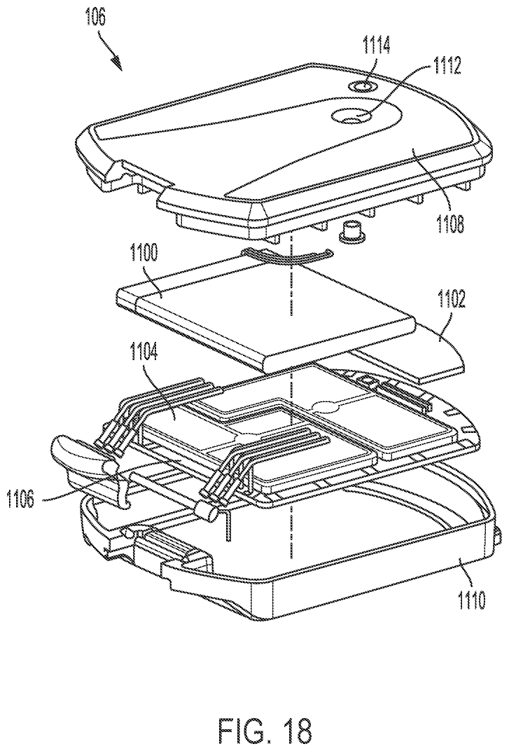

Referring back to embodiments of the wearable cardiotoxicity monitoring device 102 that include the cardiotoxicity monitoring unit 106, FIG. 18 provides an exploded view of the cardiotoxicity monitoring unit 106, according to some implementations. The exploded view of FIG. 18 illustrates various components of the cardiotoxicity monitoring unit 106. For example, the cardiotoxicity monitoring unit 106 may include a power source, such as a battery 1100. In examples, the battery 1100 may be a rechargeable lithium ion battery configured to supply power for at least one month of continuous or near-continuous recording of the one or more biosignals. The cardiotoxicity monitoring unit 106 may also include a wireless communications circuit 1102 (e.g., also implemented as wireless communications circuit 314 in FIG. 5), a radiofrequency shield 1104, a digital circuit board 1106, and/or the like. The wireless communications circuit 1102 may be a Bluetooth® unit, in some implementations. In addition to or in the alternative to the Bluetooth® unit, other components facilitating other types of communications (e.g., Wi-Fi, cellular, etc.) may be included in the cardiotoxicity monitoring unit 106. The radiofrequency shield 1104 may be implemented, for example, as a metallic cover to prevent interference with RF processing and other digital circuitry.

In implementations, the cardiotoxicity monitoring unit 106 may be configured to monitor, record, and transmit data or signals (e.g., the biosignal-based data) to the portable gateway 110 continuously (e.g., via the wireless communications circuit 1102). The cardiotoxicity monitoring unit 106 monitoring and/or recording additional data may not interrupt the transmission of already acquired data to the portable gateway 110. As such, in implementations, both the monitoring/recording and the transmission processes may occur at the same time or nearly the same time. In implementations, if the cardiotoxicity monitoring unit 106 does suspend the monitoring and/or recording of additional data while the cardiotoxicity monitoring unit 106 is transmitting already acquired data to the portable gateway 110, the cardiotoxicity monitoring unit 106 may then resume monitoring and/or recording of additional data before all of the already-acquired data has been transmitted to the portable gateway 110. To illustrate, the interruption period for the monitoring and/or recording of additional data may be less in comparison to the time it takes the cardiotoxicity monitoring unit 106 to transmit the already-acquired data (e.g., the interruption period being between about 0% to 80%, about 0% to about 60%, about 0% to about 40%, about 0% to about 20%, about 0% to about 10%, about 0% to about 5%, including values and subranges therebetween, of the monitoring and/or recording period). This moderate interruption period may facilitate the near-continuous monitoring and/or recording of additional data during transmission of already-acquired physiological data. For example, in one scenario, when a measurement time is about two minutes, any period of suspension or interruption in the monitoring and/or recording of subsequent measurement data may range from a few milliseconds to about a minute. Illustrative reasons for such suspension or interruption of data may include allowing for the completion of certain data integrity and/or other online tests of previously acquired data. In some implementations, if the previous data have problems, the cardiotoxicity monitoring unit 106 may notify the patient and/or a remote technician of the problems so that appropriate adjustments can be made.

In implementations, the cardiotoxicity monitoring unit 106 may be configured to monitor, record, and transmit some data in a continuous or near-continuous manner, as discussed above, while monitoring, recording, and transmitting some other data in a non-continuous manner (e.g., periodically, non-periodically, etc.). As an illustration, the cardiotoxicity monitoring unit 106 may be configured to record and transmit ECG data from the ECG electrodes 202 continuously or nearly continuously while data from at least one RF antenna and RF circuitry (e.g., the at least one RF antenna 304a, 304b and the RF circuitry 306) is transmitted periodically. For example, RF measurements may be taken only when the patient 100 is in a good position for transmitting and receiving RF waves, such as when the patient 100 is not moving. As such, biosignal-based data including ECG data may be transmitted to the portable gateway 110 (and, via the portable gateway 110, to the remote server 104) continuously or near-continuously as additional ECG data is being recorded, while biosignal-based data including RF data may be transmitted once the RF measuring process is completed. In implementations, monitoring and/or recording of signals by the cardiotoxicity monitoring unit 106 may be periodic and, in some implementations, may be accomplished as scheduled (e.g., periodically) without delay or latency during the transmission of already acquired data to the portable gateway 110. For example, the cardiotoxicity monitoring unit 106 may take measurements from the patient 100 and transmit the data generated from the measurements to the portable gateway 110 in a continuous manner as described above.

Additionally, in implementations, the portable gateway 110 may continuously transmit the signals provided by the cardiotoxicity monitoring unit 106 to the remote server 104. Thus, for example, the portable gateway 110 may transmit the signals from the cardiotoxicity monitoring unit 106 to the remote server 104 with little or no delay or latency.

To this end, in the context of data transmission between the wearable cardiotoxicity monitoring device 102 and the remote server 104, continuously includes continuous (e.g., without interruption) or near continuous (e.g., within one minute after completion of a measurement and/or an occurrence of an event on the cardiotoxicity monitoring unit 106). Continuity may also be achieved by repetitive successive bursts of transmission (e.g., high-speed transmission). Similarly, immediate data transmission may include data transmission occurring or done immediately or nearly immediately (e.g., within one minute after the completion of a measurement and/or an occurrence of an event on the cardiotoxicity monitoring unit 106).

Further, in the context of signal acquisition and transmission by the wearable cardiotoxicity monitoring device 102, continuously may also include uninterrupted collection of data sensed by the cardiotoxicity monitoring unit 106 with clinical continuity. In this case, short interruptions in data acquisition of up to one second several times an hour, or longer interruptions of a few minutes several times a day, may be tolerated and still seen as continuous. As to latency as a result of such a continuous scheme as described herein, the overall amount of response time (e.g., time from when an event onset is detected to when a notification regarding the event is issued) can amount, for example, from about five to fifteen minutes. As such, transmission/acquisition latency may therefore be in the order of minutes.

In implementations, the bandwidth of the link between the cardiotoxicity monitoring unit 106 and the portable gateway 110 may be larger, and in some instances, significantly larger, than the bandwidth of the acquired data to be transmitted via the link (e.g., burst transmissions). Such implementations may ameliorate issues that may arise during link interruptions, periods of reduced/absent reception, etc. In implementations, when transmission is resumed after an interruption, the resumption may be in the form of last-in-first-out (LIFO). In some implementations, the portable gateway 110 additionally may be configured to operate in a store and forward mode, where the data received from the cardiotoxicity monitoring unit 106 is first stored in an onboard memory of the portable gateway 110 and then forwarded to the remote server 104. In some implementations, the portable gateway 110 may function as a pipeline and pass through data from the cardiotoxicity monitoring unit 106 immediately to the remote server 104. Further, in implementations, the data from the cardiotoxicity monitoring unit 106 may be compressed using data compression techniques to reduce memory requirements as well as transmission times and power consumption. In some implementations, the link between the portable gateway 110 and the remote server 104 may similarly be larger, and in some instances, significantly larger, than the bandwidth of the data to be transmitted via the portable gateway 110 and the remote server 104.

Alternatively, the wearable cardiotoxicity monitoring device 102 including the cardiotoxicity monitoring unit 106 may not include the portable gateway 110. As such, the cardiotoxicity monitoring unit 106 may continuously transmit data or signals directly to the remote server 104 using similar processes as those discussed above. Additionally, in implementations, the wearable cardiotoxicity monitoring device 102 may include a garment-based monitoring device 118 that may or may not be in communication with a portable gateway 110. Similar processes may thus also be applied to the garment-based monitoring device 118 and/or the portable gateway 110 for the purpose of continuously transmitting data or signals to the remote server 104.

Referring back to FIG. 18, the components of the cardiotoxicity monitoring unit 106 (e.g., the battery 1100, communications circuit 1102, radiofrequency shield 1104, digital circuit board 1106, and/or the like) may be provided between a front cover 1108 forming an upper surface of the cardiotoxicity monitoring unit 106 and a back cover 1110 forming a bottom surface of the cardiotoxicity monitoring unit 106. For instance, the back cover 1110 may be configured to contact the adhesive patch 108, and the front cover 1108 may be configured to face away from the patient 100 such that the front cover 1108 is accessible when the cardiotoxicity monitoring unit 106 is attached to the adhesive patch 108. In some implementations, an indicator light 1112 and/or a button 1114 may be embedded into the front cover 1108 and be visible through the upper surface. The indicator light 1112 may provide feedback on the status of the cardiotoxicity monitoring unit 106 and its components, such as the charging and/or power level of the power source of the cardiotoxicity monitoring unit 106 (e.g., the battery 1100), the attachment level of the cardiotoxicity monitoring unit 106 to the adhesive patch 108, the attachment level of the adhesive patch 108 to the surface of the body to which the adhesive patch 108 is attached, etc.

The button 1114 may be configured for the patient 100 and/or a caregiver of the patient 100 to provide feedback to the cardiotoxicity monitoring unit 106 and/or, via the cardiotoxicity monitoring unit 106, to the remote server 104. For instance, the button 1114 may allow the patient 100 and/or caregiver to activate or deactivate the cardiotoxicity monitoring unit 106. In some implementations, the button 1114 may be used to reset the cardiotoxicity monitoring unit 106, as well as pair the cardiotoxicity monitoring unit 106 to the portable gateway 110 and initiate communication with the portable gateway 110. In some implementations, the button 1114 may allow a user to set the cardiotoxicity monitoring unit 106 in an “airplane mode,” for example, by deactivating any wireless communication (e.g., Wi-Fi, Bluetooth®, etc.) with external devices and/or servers, such as the portable gateway 110 and/or the remote server 104. In implementations, the button 1114 may allow the patient 100 to report experiencing a symptom, as described above.

Referring back to FIG. 5, the example cardiotoxicity monitoring unit 106 includes additional components to those discussed above with reference to FIG. 5. In implementations, as shown in FIG. 5, the cardiotoxicity monitoring unit 106 includes one or more external interfaces, cither connected to or embedded in the cardiotoxicity monitoring unit 106. For example, the cardiotoxicity monitoring unit 106 may include the button or switch 1114 for activating the cardiotoxicity monitoring unit 106, deactivating the cardiotoxicity monitoring unit 106, pairing the cardiotoxicity monitoring unit 106 with the portable gateway 110, receiving a patient input related to a symptom, and/or the like. In implementations, the cardiotoxicity monitoring unit 106 may also include the indicator light 1112 and a buzzer 324 for providing light and/or audio feedback to a user of the cardiotoxicity monitoring unit 106 (e.g., in response to the patient 100 activating the button 1114 or tapping the cardiotoxicity monitoring unit 106 to record that the patient 100 is experiencing symptoms suspected to be related to an arrhythmia).

Further, in some embodiments, the cardiotoxicity monitoring unit 106 may be connectable to the ECG pads or electrodes 202 coupled to the patient 100 (e.g., connectable to the ECG pads 202 embedded in the adhesive patch 108) and to a charger, such as charger 112, via a charging link 320. For instance, the back cover 1110 of the cardiotoxicity monitoring unit 106 may implement the charging link 320 via metal contacts when the cardiotoxicity monitoring unit 106 is attached to a charging power source like the charger 112. The cardiotoxicity monitoring unit 106 may use the same metal contacts to link to the ECG pads 202 when the cardiotoxicity monitoring unit 106 is attached to the adhesive patch 108. As discussed above, the ECG circuits 300 may then receive signals from the ECG pads 202 when the cardiotoxicity monitoring unit 106 is attached to the adhesive patch 108. Alternatively or additionally, in implementations the charging link 320 may be implemented through an inductive circuit configured to charge the cardiotoxicity monitoring unit 106 via a wireless inductive charging. As shown in FIG. 5, the charging link 320 may be coupled to a power management circuit 322 (e.g., when the cardiotoxicity monitoring unit 106 is attached to the charger 112, when the cardiotoxicity monitoring unit 106 is placed in proximity to an inductive charging pad), where the power management circuit 322 is configured to charge an onboard power source such as the battery 1100.

Referring back to embodiments of the wearable cardiotoxicity monitoring device 102 including the garment-based monitoring device 118, FIG. 8 illustrates additional components of the medical device controller 500 that may be used in the operation of the garment-based monitoring device 118. In implementations, the user interface 510 may include one or more physical interface devices, such as input devices, output devices, and combination input/output devices, and a software stack configured to drive operation of the devices. These user interface elements may render visual, audio, and/or tactile content. Thus, the user interface 510 may receive inputs and/or provide outputs, thereby enabling a user to interact with the medical device controller 500.

The medical device controller 500 can also include at least one battery 512 configured to provide power to one or more components integrated in the medical device controller 500. The battery 512 can include a rechargeable multi-cell battery pack. In one example implementation, the battery 512 can include three or more cells (e.g., 2200 mA lithium ion cells) that provide electrical power to the other device components within the medical device controller 500. For example, the battery 512 can provide its power output in a range of between 20 mA to 1000 mA (e.g., 40 mA) output and can support 24 hours, 48 hours, 72 hours, or more, of runtime between charges. In certain implementations, the battery capacity, runtime, and type (e.g., lithium ion, nickel-cadmium, or nickel-metal hydride) can be changed to best fit the specific application of the medical device controller 500.

The alarm manager 516 can be implemented using hardware or a combination of hardware and software. For instance, in some examples, the alarm manager 516 can be implemented as a software component that is stored within the data storage 506 and executed by the processor 518. In this example, the instructions included in the alarm manager 516 can cause the processor 518 to configure alarm profiles and notify intended recipients using the alarm profiles. In other examples, the alarm manager 516 can be an application-specific integrated circuit (ASIC) that is coupled to the processor 518 and configured to manage alarm profiles and notify intended recipients using alarms specified within the alarm profiles. Thus, examples of the alarm manager 516 are not limited to a particular hardware or software implementation.

The therapy delivery circuit 530 can be coupled to the therapy electrodes 404 configured to provide therapy to the patient 100. For example, the therapy delivery circuit 530 can include, or be operably connected to, circuitry components that are configured to generate and provide an electrical therapeutic shock. The circuitry components can include, for example, resistors, capacitors, relays and/or switches, electrical bridges such as an H-bridge (e.g., including a plurality of insulated gate bipolar transistors or IGBTs), voltage and/or current measuring components, and other similar circuitry components arranged and connected such that the circuitry components work in concert with the therapy delivery circuit 530 and under the control of one or more processors (e.g., processor 518) to provide, for example, one or more pacing, defibrillation, or cardioversion therapeutic pulses. In implementations, pacing pulses can be used to treat cardiac arrhythmias such as bradycardia (e.g., less than 30 beats per minute) and tachycardia (e.g., more than 150 beats per minute) using, for example, fixed rate pacing, demand pacing, anti-tachycardia pacing, and the like. Defibrillation or cardioversion pulses can be used to treat ventricular tachycardia and/or ventricular fibrillation.

In implementations, the therapy delivery circuit 530 includes a first high-voltage circuit connecting a first pair of the therapy electrodes 404 and a second high-voltage circuit connecting a second pair of the therapy electrodes 404 such that a first biphasic therapeutic pulse is delivered via the first high-voltage circuit and a second biphasic therapeutic pulse is delivered via the second high-voltage circuit. In implementations, the second high-voltage circuit is configured to be electrically isolated from the first high-voltage circuit. In implementations, the therapy delivery circuit 530 includes a capacitor configured to be selectively connected to the first high-voltage circuit and/or the second high-voltage circuit. As such, the first high-voltage circuit may powered by the capacitor when the capacitor is selectively connected to the first high-voltage circuit, and the second high-voltage circuit may be powered by the capacitor when the capacitor is selectively connected to the second high-voltage circuit. In implementations, the therapy delivery circuit 530 includes a first capacitor electrically connected to the first high-voltage circuit and a second capacitor electrically connected to the second high-voltage circuit.

The capacitors can include a parallel-connected capacitor bank consisting of a plurality of capacitors (e.g., two, three, four, or more capacitors). In some examples, the capacitors can include a single film or electrolytic capacitor as a series connected device including a bank of the same capacitors. These capacitors can be switched into a series connection during discharge for a defibrillation pulse. For example, four capacitors of approximately 140 μF or larger, or four capacitors of approximately 650 μF can be used. The capacitors can have a 1600 volts DC (VDC) or higher rating for a single capacitor, or a surge rating between approximately 350 to 500 VDC for paralleled capacitors, and can be charged in approximately 15 to 30 seconds from a battery pack.

For example, each defibrillation pulse can deliver between 60 J to 180 J of energy. In some implementations, the defibrillating pulse can be a biphasic truncated exponential waveform, whereby the signal can switch between a positive and a negative portion (e.g., charge directions). This type of waveform can be effective at defibrillating patients at lower energy levels when compared to other types of defibrillation pulses (e.g., such as monophasic pulses). For example, an amplitude and a width of the two phases of the energy waveform can be automatically adjusted to deliver a precise energy amount (e.g., 150 J) regardless of the patient's body impedance. The therapy delivery circuit 530 can be configured to perform the switching and pulse delivery operations, e.g., under control of the processor 518. As the energy is delivered to the patient 100, the amount of energy being delivered can be tracked. For example, the amount of energy can be kept to a predetermined constant value even as the pulse waveform is dynamically controlled based on factors, such as the patient's body impedance, while the pulse is being delivered.

In certain examples, the therapy delivery circuit 530 can be configured to deliver a set of cardioversion pulses to correct, for example, an improperly beating heart. When compared to defibrillation as described above, cardioversion typically includes a less powerful shock that is delivered at a certain frequency to mimic a heart's normal rhythm.

In implementations, the cardiac event detector 514 can be configured to monitor the patient's ECG signal for an occurrence of a cardiac event such as an arrhythmia or other similar cardiac event. The cardiac event detector 514 can be configured to operate in concert with the processor 518 to execute one or more methods that process received ECG signals from, for example, the ECG sensing electrodes 202 and determine the likelihood that the patient 100 is experiencing a cardiac event, such as a treatable arrhythmia. The cardiac event detector 514 can be implemented using hardware or a combination of hardware and software. For instance, in some examples, the cardiac event detector 514 can be implemented as a software component that is stored within the data storage 506 and executed by the processor 518. In this example, the instructions included in the cardiac event detector 514 can cause the processor 518 to perform one or more methods for analyzing a received ECG signal to determine whether an adverse cardiac event is occurring, such as a treatable arrhythmia. In other examples, the cardiac event detector 514 can be an application-specific integrated circuit (ASIC) that is coupled to the processor 518 and configured to monitor ECG signals for adverse cardiac event occurrences. Thus, examples of the cardiac event detector 514 are not limited to a particular hardware or software implementation.

In response to the cardiac event detector 514 determining that the patient 100 is experiencing a treatable arrhythmia, the processor 518 is configured to deliver a cardioversion/defibrillation shock to the patient 100 via the therapy electrodes 404. In implementations, the alarm manager 516 can be configured to manage alarm profiles and notify one or more intended recipients of events, where an alarm profile includes a given event and the intended recipients who may have in interest in the given event. These intended recipients can include external entities, such as users (e.g., patients, physicians and other caregivers, a patient's loved one, monitoring personnel), as well as computer systems (e.g., monitoring systems or emergency response systems, which may be included in the remote server 104 or may be implemented as one or more separate systems). For example, when the processor 518 determines using data from the ECG sensing electrodes 202 that the patient 100 is experiencing a treatable arrhythmia, the alarm manager 516 may issue an alarm via the user interface 510 that the patient 100 is about to experience a defibrillating shock. The alarm may include auditory, tactile, and/or other types of alerts. In some implementations, the alerts may increase in intensity over time, such as increasing in pitch, increasing in volume, increasing in frequency, switching from a tactile alert to an auditory alert, and so on. Additionally, in some implementations, the alerts may inform the patient 100 that the patient 100 can abort the delivery of the defibrillating shock by interacting with the user interface 510. For instance, the patient 100 may be able to press a user response button or user response buttons on the user interface 510, after which the alarm manager 516 will cease issuing an alert and the medical device controller 500 will no longer prepare to deliver the defibrillating shock.

FIG. 19 illustrates another example of a wearable cardiotoxicity monitoring device 102. More specifically, FIG. 19 shows a hospital wearable defibrillator 1200 that is external, ambulatory, and wearable by the patient 100. In some implementations, hospital wearable defibrillator 1200 can be configured to also provide pacing therapy, e.g., to treat bradycardia, tachycardia, and asystole conditions. The hospital wearable defibrillator 1200 can include one or more ECG sensing electrodes 1202a, 1202b, 1202c (e.g., collectively ECG sensing electrodes 1202), therapy electrodes 1204a and 1204b (e.g., collectively therapy electrodes 1204), a medical device controller 1206, and a connection pod 1208. For example, each of these components can be structured and function as similar components of the embodiments of the garment-based monitoring device 118 discussed above with reference to FIGS. 6-8. In implementations, the electrodes 1202 and 1204 can include disposable adhesive electrodes. For example, the electrodes 1202 and 1204 can include sensing and therapy components disposed on separate sensing and therapy electrode adhesive patches. In implementations, both sensing and therapy components can be integrated and disposed on a same electrode adhesive patch that is then attached to the patient 100.

As an illustration, the front adhesively attachable therapy electrode 1204a attaches to the front of the patient's torso to deliver pacing or defibrillating therapy. Similarly, the back adhesively attachable therapy electrode 1204b attaches to the back of the patient's torso. In an example scenario, at least three ECG adhesively attachable sensing electrodes 1202 can be attached at least above the patient's chest near the right arm (e.g., electrode 1202a), above the patient's chest near the left arm (e.g., electrode 1202b), and towards the bottom of the patient's chest (e.g., electrode 1202c) in a manner prescribed by a trained professional. In implementations, the hospital wearable defibrillator 1200 may include additional adhesive therapy electrodes 1204, and/or the patches shown in FIG. 19 may include additional therapy electrodes 1204 on them, such that at least two vectors may be formed between the therapy electrodes 1204 of the hospital wearable defibrillator 1200, as described above with reference to the garment-based monitoring device 118 of FIGS. 6-8.

In implementations, the medical device controller 1206 may be configured to function similarly to the medical device controller 406 and medical device controller 500 discussed above with respect to the garment-based monitoring device 118. As shown in FIG. 19, the medical device controller 1206 may include a user interface 1210 configured to communicate information with the patient 100. In examples, the patient 100 being monitored by a hospital wearable defibrillator and/or pacing device 1200 may be confined to a hospital bed or room for a significant amount of time (e.g., 75% or more of the patient's stay in the hospital). As a result, a user interface 1210 can be configured to interact with a user other than the patient 100 (e.g., a technician, a clinician or other caregiver) for device-related functions such as initial device baselining (e.g., including performing a baselining therapy session), setting and adjusting patient parameters, and changing the device batteries.

FIG. 20 illustrates another example of a wearable cardiotoxicity monitoring device 102. As shown in FIG. 20, the wearable cardiotoxicity monitoring device 102 may be or include an adhesive assembly 1300. The adhesive assembly 1300 includes a contoured pad 1302 and a housing 1304 configured to form a watertight seal with the contoured pad 1302. In implementations, the housing 1304 is configured to house electronic components of the adhesive assembly 1300, such as electronic components similar to components described above with respect to the cardiotoxicity monitoring unit 106 of FIG. 5 and/or the medical device controller 406 of FIGS. 6 and 7 (and the medical device controller 500 of FIG. 8). The adhesive assembly 1300 includes a conductive adhesive layer 1306 configured to adhere the adhesive assembly 1300 to a skin surface 1308 of the patient 100. The adhesive layer 1306 may include, for example, a water-vapor permeable conductive adhesive material, such as a material selected from the group consisting of an electro-spun polyurethane adhesive, a polymerized microemulsion pressure sensitive adhesive, an organic conductive polymer, an organic semi-conductive conductive polymer, an organic conductive compound, and a semi-organic conductive compound, and combinations thereof.

The adhesive assembly 1300 also includes at least one therapy electrode 1310 integrated with the contoured pad 1302. In implementations, the adhesive assembly 1300 may include a therapy electrode 1310 that forms a vector with another therapy electrode disposed on another adhesive assembly 1300 adhered to the patient's body and/or with a separate therapy electrode adhered to the patient's body (e.g., similar to therapy electrodes 1204 shown in FIG. 19). The adhesive assembly 1300 may also include one or more ECG sensing electrodes 1312 integrated with the contoured pad 1302 (e.g., ECG sensing electrodes 1312a and 1312b). In implementations, the adhesive assembly 1300 may alternatively or additionally be in electronic communication with a separate ECG sensing electrode, such as an adhesive sensing electrode adhered to the patient's body. In examples, as shown in FIG. 20, the therapy electrode(s) 1310 and ECG sensing electrode(s) 1312 may be formed within the contoured pad 1302 such that a skin-contacting surface of each component is coplanar with or protrudes from the patient-contacting face of the contoured pad 1302. Examples of a wearable cardiotoxicity monitoring device 102 including an adhesive assembly 1300 are described in U.S. patent application Ser. No. 16/585,344, entitled “Adhesively Coupled Wearable Medical Device,” filed on Sep. 27, 2019, which is hereby incorporated by reference in its entirety.

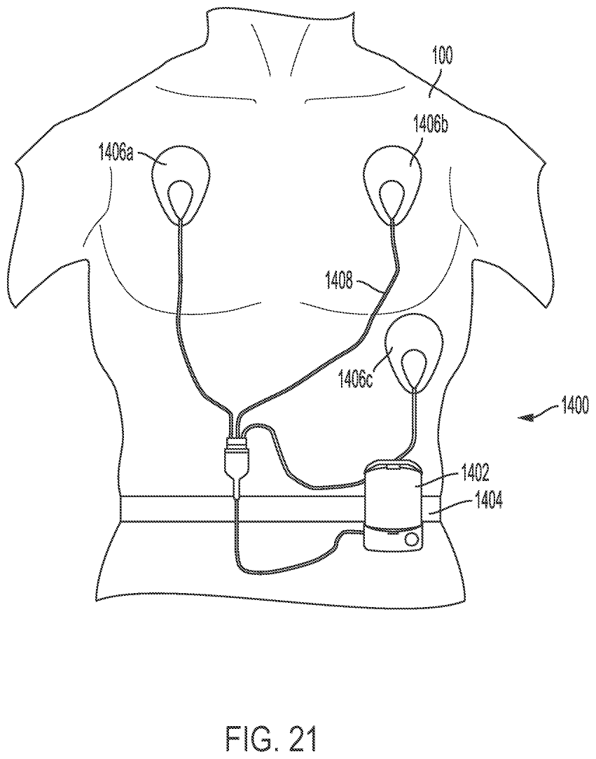

FIG. 21 illustrates another example of a wearable cardiotoxicity monitoring device 102. As shown in FIG. 21, a wearable cardiotoxicity monitoring device 102 may include a belted monitoring device 1400 that is external, ambulatory, and wearable by the patient 100. In implementations, the belted monitoring device 1400 may include a medical device controller 1402 configured to be worn mounted on a belt 1404 around the patient's torso. As such, the belted monitoring device 1400 may be configured similarly to the hospital wearable defibrillator 1200 shown in FIG. 20. In implementations, the belted monitoring device 1400 may instead include a medical device controller 1402 integrated into the belt 1404. In such implementations, the belt 1404 includes a number of modules housing the circuitry of the medical device controller 1402 such that the patient 100 does not need to wear a separate medical device controller 1402. Regardless of the implementation, the medical device controller 1402 implemented either as a separate unit or integrated into the belt 1404 may be configured to function similarly to the medical device controller 406 described above with reference to FIG. 7 and the medical device controller 500 described above with reference to FIG. 8.

Similar to the hospital wearable defibrillator 1200, the belted monitoring device 1400 can include adhesive electrodes 1406a, 1406b, 1406c (e.g., collectively adhesive electrodes 1406) configured to be attached to the patient's skin. For example, the adhesive electrodes 1406 may be disposable adhesive electrodes in a wired connection 1408 with the medical device controller 1402 (or, in implementations, with the belt 1404 including the circuitry of the medical device controller 1402). Alternatively, at least some of the adhesive electrodes 1406 may be wirelessly connected to the medical device controller 1402 (or, in implementations, with the belt 1404 including the circuitry of the medical device controller 1402). For instance, the adhesive electrodes 1406 may be configured to communicate via Bluetooth® with the medical device controller 1402 (or the belt 1404). In implementations, at least some of the adhesive electrodes 1406 may include both sensing and therapy components integrated into the same electrode adhesive patch that is attached to the patient. In implementations, at least some of the adhesive electrodes 1406 may be a dedicated sensing electrode or a dedicated therapy electrode. For example, adhesive electrodes 1406a and 1406c may be dedicated therapy electrodes. In implementations, the belted monitoring device 1400 may include additional adhesive electrodes 1406 include sensing and/or therapy components configured to form additional sensing and/or therapy electrode vectors.

Additionally, in implementations, the belted monitoring device 1400 may be configured as a monitoring only device. As such, the adhesive electrodes 1406 may only include sensing electrodes, and the medical device controller 1402 may be configured to monitor the patient 100 without providing any treatment. Alternatively, the therapeutic components of the belted monitoring device 1400 may be disconnectable or be configured to be switched off such that the belted monitoring device 1400 can operate in a monitoring only mode (e.g., similar to the medical device controller 500 described above with reference to FIG. 8).

Although the subject matter contained herein has been described in detail for the purpose of illustration, such detail is solely for that purpose and that the present disclosure is not limited to the disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

Other examples are within the scope and spirit of the description and claims. Additionally, certain functions described above can be implemented using software, hardware, firmware, hardwiring, or combinations of any of these. Features implementing functions can also be physically located at various positions, including being distributed such that portions of functions are implemented at different physical locations.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. Those skilled in the art will readily appreciate that all parameters, dimensions, materials, and configurations described herein are meant to be an example and that the actual parameters, dimensions, materials, and/or configurations will depend upon the specific application or applications for which the inventive teachings is/are used.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

1-113. (canceled)

114. A wearable cardiotoxicity monitoring system for monitoring cardiotoxicity biosignal markers in oncology patients, comprising:
a wearable cardiotoxicity monitoring device configured for long-term, continuous wear by a patient, comprising
a plurality of externally applied biosignal sensors configured to sense one or more biosignals from the patient, the plurality of externally applied biosignal sensors comprising
a plurality of ECG electrodes configured to sense one or more electrical signals indicative of ECG activity from a skin surface of the patient, and
at least one non-ECG physiological sensor configured to sense a non-ECG biosignal of the patient, and
a controller operationally coupled to the plurality of externally applied biosignal sensors, the controller configured to
transmit biosignal-based data based on the one or more sensed biosignals to a remote server; and
the remote server in communication with the wearable cardiotoxicity monitoring device, comprising
a memory implemented in a non-transitory media, and
a processor in communication with the memory, the processor configured to
receive the biosignal-based data from the wearable cardiotoxicity monitoring device;
analyze the biosignal-based data from the wearable cardiotoxicity monitoring device to identify at least one cardiotoxicity biosignal marker associated with heart failure caused by at least one of chemotherapy cardiotoxicity or radiation therapy cardiotoxicity;
determine a current status of cardiotoxicity in the patient based on the at least one cardiotoxicity biosignal marker; and
generate an output based on the current status of cardiotoxicity in the patient.

115. The wearable cardiotoxicity monitoring system of claim 114, wherein the wearable cardiotoxicity monitoring device further comprises a garment configured to be worn around the patient's torso.

116. The wearable cardiotoxicity monitoring system of claim 115, wherein at least a portion of the plurality of externally applied biosignal sensors are configured to be removably mounted into the garment.

117. The wearable cardiotoxicity monitoring system of claim 114, wherein the wearable cardiotoxicity monitoring device further comprises one or more therapy electrodes configured to deliver one or more therapeutic shocks to the patient.

118. The wearable cardiotoxicity monitoring system of claim 117, wherein the controller is further configured to
detect a treatable cardiac arrhythmia in the patient; and
generate the one or more therapeutic shocks for delivery to the patient via the one or more therapy electrodes based on detecting the treatable cardiac arrhythmia in the patient.

119. The wearable cardiotoxicity monitoring system of claim 114, wherein the at least one non-ECG physiological sensor comprises at least one of a cardiovibration sensor configured to sense biosignals comprising cardiovibrations of the patient;
a respiration sensor configured to sense biosignals indicative of a respiration rate of the patient;
a radiofrequency (RF) sensor configured to sense biosignals indicative of a thoracic fluid level of the patient; or
a blood pressure sensor configured to sense biosignals indicative of a blood pressure of the patient.

120. The wearable cardiotoxicity monitoring system of claim 114, wherein the biosignal-based data comprises an ECG of the patient, and the at least one cardiotoxicity biosignal marker comprises at least one ECG marker associated with heart failure caused by the at least one of chemotherapy cardiotoxicity or radiation therapy cardiotoxicity.

121. The wearable cardiotoxicity monitoring system of claim 120, wherein the at least one ECG marker associated with heart failure caused by the at least one of chemotherapy cardiotoxicity or radiation therapy cardiotoxicity comprises a PR interval duration.

122. The wearable cardiotoxicity monitoring system of claim 120, wherein the at least one ECG marker associated with heart failure caused by the at least one of chemotherapy cardiotoxicity or radiation therapy cardiotoxicity comprises a QRS complex duration.

123. The wearable cardiotoxicity monitoring system of claim 120, wherein the at least one ECG marker associated with heart failure caused by the at least one of chemotherapy cardiotoxicity or radiation therapy cardiotoxicity comprises a QT interval duration.

124. The wearable cardiotoxicity monitoring system of claim 114, wherein the wearable cardiotoxicity monitoring device further comprises at least one additional externally applied sensor configured to sense one or more additional signals associated with the patient; and
wherein the controller is further configured to transmit additional sensor data based on the one or more additional signals associated with the patient to the remote server.

125. The wearable cardiotoxicity monitoring system of claim 124, wherein the processor is further configured to
receive the additional sensor data from the wearable cardiotoxicity monitoring device;
analyze the additional sensor data from the wearable cardiotoxicity monitoring device to identify at least one additional cardiotoxicity marker associated with heart failure caused by the at least one of chemotherapy cardiotoxicity or radiation therapy cardiotoxicity; and
determine the current status of cardiotoxicity in the patient further based on the at least one additional cardiotoxicity marker.

126. The wearable cardiotoxicity monitoring system of claim 125, wherein the at least one additional externally applied sensor comprises an accelerometer configured to sense one or more motion signals associated with the patient; and
wherein the additional sensor data based on the one or more additional signals comprises accelerometer data based on the one or more motion signals.

127. The wearable cardiotoxicity monitoring system of claim 114, wherein the processor is configured to determine the current status of cardiotoxicity in the patient based on the at least one cardiotoxicity biosignal marker by determining whether the at least one cardiotoxicity biosignal marker transgresses at least one predetermined threshold.

128. The wearable cardiotoxicity monitoring system of claim 127, wherein determining whether the at least one cardiotoxicity biosignal marker transgresses the at least one predetermined threshold comprises determining whether a first cardiotoxicity biomarker transgresses a first predetermined threshold associated with a first, lower heart failure severity and whether the first cardiotoxicity biomarker transgresses a second predetermined threshold associated with a second, higher heart failure severity.

129. The wearable cardiotoxicity monitoring system of claim 114, wherein the controller is further configured to, before the patient begins at least one of chemotherapy or radiation therapy, transmit baseline biosignal-based data based on the one or more sensed biosignals to the remote server.

130. The wearable cardiotoxicity monitoring system of claim 129, wherein the processor is further configured to
receive the baseline biosignal-based data from the wearable cardiotoxicity monitoring device;
analyze the baseline biosignal-based data from the wearable cardiotoxicity monitoring device to identify at least one baseline cardiotoxicity biosignal marker associated with a baseline heart failure status before application of at least one of chemotherapy or radiation therapy to the patient; and
determine a baseline status of cardiotoxicity in the patient based on the at least one baseline cardiotoxicity biosignal marker.

131. The wearable cardiotoxicity monitoring system of claim 130, wherein the processor is further configured to generate a baseline output comprising a cardiac risk assessment for the patient based on the baseline status of cardiotoxicity in the patient.

132. The wearable cardiotoxicity monitoring system of claim 114, wherein the controller is further configured to, after the patient finishes at least one of chemotherapy or radiation therapy, transmit post-therapy biosignal-based data based on the one or more sensed biosignals to the remote server.

133. The wearable cardiotoxicity monitoring system of claim 132, wherein the processor is further configured to
receive the post-therapy biosignal-based data from the wearable cardiotoxicity monitoring device;
analyze the post-therapy biosignal-based data from the wearable cardiotoxicity monitoring device to identify at least one post-therapy cardiotoxicity biosignal marker associated with heart failure caused by the at least one of chemotherapy cardiotoxicity or radiation therapy cardiotoxicity; and
determine a post-therapy status of cardiotoxicity in the patient based on the at least one post-therapy cardiotoxicity biosignal marker.
